(19) United States
(12) Patent Application Publication
(10) Pub. No.: US 2013/0165440 A1
(43) Pub. Date: Jun. 27, 2013

(54) JAK1 Inhibitors

(71) Applicants: Neel Kumar Anand, Burlingame, CA (US); S. David Brown, San Carlos, CA (US); Zerom Tesfai, Castro Valley, CA (US); Cristiana A. Zaharia, Redwood City, CA (US)

(72) Inventors: Neel Kumar Anand, Burlingame, CA (US); S. David Brown, San Carlos, CA (US); Zerom Tesfai, Castro Valley, CA (US); Cristiana A. Zaharia, Redwood City, CA (US)

(73) Assignee: EXELIXIS, INC., South San Francisco, CA (US)

(21) Appl. No.: 13/821,435

(22) PCT Filed: Sep. 13, 2011
(86) PCT No.: PCT/US11/51408

§ 371 Date: Mar. 7, 2013

Related U.S. Application Data
(60) Provisional application No. 61/382,906, filed on Sep. 14, 2010.

(51) Int. Cl. C07D 237/34; C07D 403/10; C07D 409/10; C07D 215/44; C07D 409/04; C07D 403/12; C07D 413/12; C07D 417/10; C07D 471/04

(52) U.S. Cl. CPC C07D 237/34; C07D 417/10; C07D 403/10; C07D 409/10; C07D 471/04; C07D 409/04; C07D 403/12; C07D 413/12; C07D 215/44
USPC 514/234.5; 544/237; 514/248; 544/236; 544/119; 546/160; 514/313; 435/184

(57) ABSTRACT

JAK1 inhibitors of structural formula (I), wherein Ar¹, Ar², Q, W, X, Y, and Z are defined in the specification, pharmaceutically acceptable salts thereof, compositions thereof, and use of the compounds and compositions for treating diseases. The invention also comprises use of the compounds in and for the manufacture of medicaments, particularly for treating diseases.

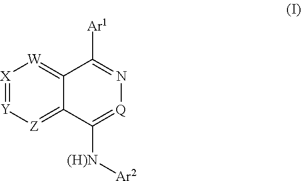

### BACKGROUND OF THE INVENTION

1. Field of the Invention

The invention relates to inhibitors of Janus Kinase 1 (JAK1), compositions comprising them, methods of making the compounds and compositions and using them for the treatment of diseases JAK1 mediates or is implicated in.

2. Summary of the Related Art

Janus kinases (JAKs), a small family of non-receptor protein tyrosine kinases, along with signal transducers, and activators of transcription (STATs) are crucial intracellular elements in cytokine signaling. There are four JAK family members, JAK1, JAK2, JAK3, and TYK2. The JAK isoforms vary in function, and therefore there exists a need in the art for isoform-specific inhibitors that can reduce undesired effects from the administration of generalized JAK inhibitors.

JAK1 plays a key role in types I and II interferon signaling and elicits signals from the interleukin-2, interleukin-4, gp130 and class II receptor families. As such, small molecule inhibition of JAK1 may intervene in the signaling pathways involved in oncology, inflammation and autoimmune diseases.

Thus selective JAK1 inhibitors may be effective in treating cancer, including, but not limited to, carcinomas, sarcomas, lymphomas, leukemias, myelomas, germ cell tumors, blastomas, tumors of the central and peripheral nervous system and other tumors including melanomas, seminoma and Kaposi's sarcoma and the like. Selective JAK1 inhibitors may also be effective in treating immune and/or inflammatory disorders which include, but are not limited to, acquired immunodeficiency syndrome (AIDS), Addison's disease, adult respiratory distress syndrome, allergies, ankylosing spondylitis, amyloidosis, asthma, autoimmune hemolytic anemia, autoimmune thyroiditis, Crohn's disease, episodic lymphopenia with lymphocytotoxins, erythroblastosis fetalis, Goodpasture's syndrome, Graves' disease, Hashimoto's thyroiditis, hypereosinophilia, irritable bowel syndrome and other interbowel diseases, Lupus, myasthenia gravis, myocardial or pericardial inflammation, pancreatitis, polymyositis, psoriasis, Reiter's syndrome, scleroderma, systemic analphylaxis, lucerative colitis, nephritis (including glomerulonephritis), gout, arthritis (such as rheumatoid arthritis and osteoarthritis), erythema, dermatitis, dermatomyositis, bronchitis, cholecystitis, and gastritis. To date, there are no examples of highly selective JAK1 inhibitors.

### SUMMARY OF THE INVENTION

The present invention comprises JAK1 inhibitors of structural formula I,

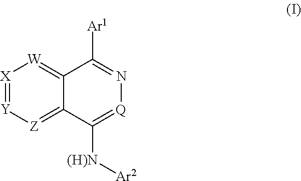

wherein Ar¹, Ar², Q, W, X, Y, and Z are defined herein below, and pharmaceutically acceptable salts thereof. The invention further comprises compositions comprising the compounds and/or pharmaceutically acceptable salts thereof. The invention also comprises use of the compounds and compositions for treating diseases. The invention also comprises use of the compounds in and for the manufacture of medicaments, particularly for treating diseases.

The invention also comprises use of the compounds and compositions for treating diseases in which JAK1 is a mediator or is implicated. The invention also comprises use of the compounds in and for the manufacture of medicaments, particularly for treating diseases in which JAK1 is a mediator or is implicated.

### DETAILED DESCRIPTION OF THE INVENTION

One aspect of the invention relates to a compound of Formula I,

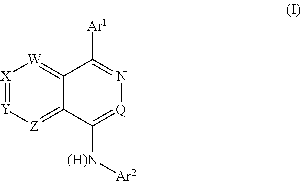

or a pharmaceutically acceptable salt thereof, wherein:

Ar¹ is phenyl optionally substituted with 1-2 R¹ groups or optionally fused to a 5-6 membered heterocyclyl, heterocyclyl, or heteroaryl optionally substituted with 1-2 R² groups;

Ar² is phenyl optionally substituted with 1-3 R⁵ groups;

each R¹ is independently halo, alkyl, —C(O)OR³, —C(O)R³, —C(O)N(H)alkylR⁴, —N(H)C(O)alkyl, —C(O)N(R³)(R⁴), —SO₂R³, heteroaryl optionally substituted with R³ or —NR³R⁷, or heterocyclyl substituted with oxo;

each R² is independently —N(R³)(R⁴), -alkylN(R3)(R⁴), oxo, alkyl, —C(O)R³, or —C(O)OR³;

R³ is H or alkyl;

R⁴ is H or alkyl optionally substituted with heterocyclyl;

each R⁵ is independently halo, —CN, —C(O)OR³, R⁶, —OR⁶, —N(R³)R⁶, alkyl optionally substituted with 1-3 halo, alkoxy optionally substituted with 1-3 halo, or heterocyclyl optionally substituted with R³;

R⁶ is alkyl optionally substituted with —NR³R⁷;

R² is H or alkyl;

Q is C(H) or N;

W is C(H) or N;

X is C(H) or N;

Y is C(H) or N; and

Z is C(H) or N;

provided the compound is not

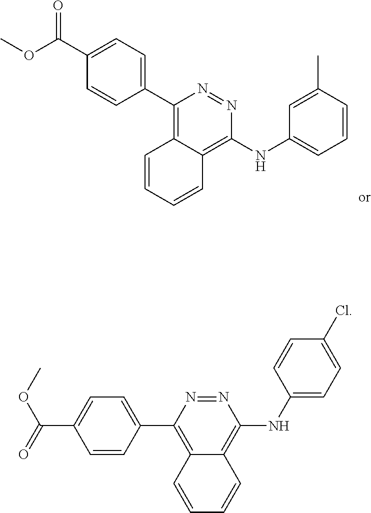 [SMILES: COC(=O)c1ccc(-c2nnc(Nc3cccc(C)c3)c3ccccc23)cc1 ; COC(=O)c1ccc(-c2nnc(Nc3ccc(Cl)cc3)c3ccccc23)cc1]

In another embodiment, the invention relates to a compound of Formula I,

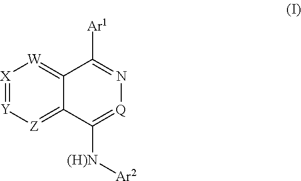

or a pharmaceutically acceptable salt thereof, wherein:

Ar¹ is phenyl optionally substituted with 1-2 R¹ groups, or heteroaryl optionally substituted with 1-2 R² groups;

Ar² is phenyl optionally substituted with 1-3 R⁵ groups;

each R¹ is independently —C(O)OR³, —C(O)R³, —C(O)N(H)alkylR⁴, —N(H)C(O)alkyl, —C(O)N(R³)(R⁴), or —SO₂R³;

each R² is independently —N(R³)(R⁴), alkyl, or —C(O)OR³;

R³ is H or alkyl;

R⁴ is H or alkyl optionally substituted with heterocyclyl;

each R⁵ is independently halo, —CN, —C(O)OR³, R⁶, —OR⁶, —N(R³)R⁶, alkyl optionally substituted with 1-3 halo, alkoxy optionally substituted with 1-3 halo, or heterocyclyl optionally substituted with R³;

R⁶ is alkyl optionally substituted with —NR³R⁷;

R⁷ is H or alkyl;

Q is C(H) or N;

W is C(H) or N;

X is C(H) or N;

Y is C(H) or N; and

Z is C(H) or N,

provided the compound is not

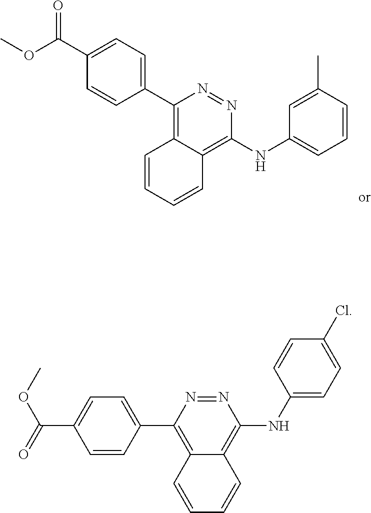 [SMILES: COC(=O)c1ccc(-c2nnc(Nc3cccc(C)c3)c3ccccc23)cc1 ; COC(=O)c1ccc(-c2nnc(Nc3ccc(Cl)cc3)c3ccccc23)cc1]

In another embodiment, the invention relates to a compound of Formula I,

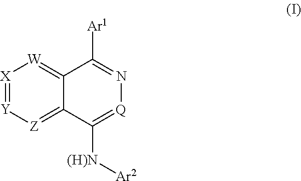

or a pharmaceutically acceptable salt thereof, wherein:

Ar¹ is phenyl optionally substituted with 1-2 R¹ groups or heteroaryl optionally substituted with 1-2 R² groups;

Ar² is phenyl optionally substituted with 1-3 R⁵ groups;

each R¹ is independently —C(O)OR³, —C(O)R³, —C(O)N(H)alkylR⁴; —N(H)C(O)alkyl, —C(O)N(R³)(R⁴), —SO₂R³, heteroaryl optionally substituted with —NR³R⁷, or heterocyclyl substituted with oxo;

each R² is independently —N(R³)(R⁴), oxo, alkyl, —C(O)R³, or —C(O)OR³;

R³ is H or (C₁-C₃)alkyl;

R⁴ is H or (C₁-C₃)alkyl optionally substituted with a 5-6 membered heterocyclyl;

each R⁵ is independently halo, —CN, —C(O)OR³, R⁶, —OR⁶, —N(R³)R⁶, alkyl optionally substituted with 1-3 halo, alkoxy optionally substituted with 1-3 halo, or heterocyclyl optionally substituted with R³;

R⁶ is alkyl optionally substituted with —NR³R⁷;

R⁷ is H or alkyl;

Q is C(H) or N;

W is C(H) or N;

X is C(H) or N;

Y is C(H) or N; and

Z is C(H) or N;

provided that only one of W, X, Y or Z can be N, and provided that the compound is not

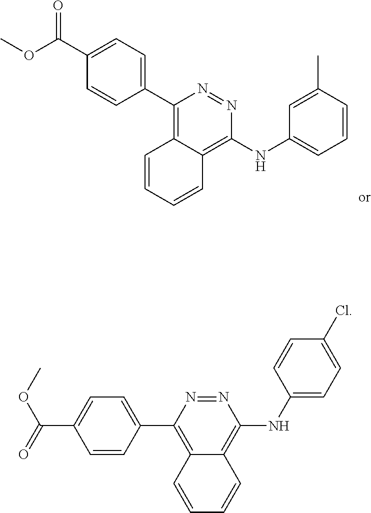 [SMILES: COC(=O)c1ccc(-c2nnc(Nc3cccc(C)c3)c3ccccc23)cc1 ; COC(=O)c1ccc(-c2nnc(Nc3ccc(Cl)cc3)c3ccccc23)cc1]

In certain embodiments, the JAK1 inhibitors of the present invention are of Formula IA:

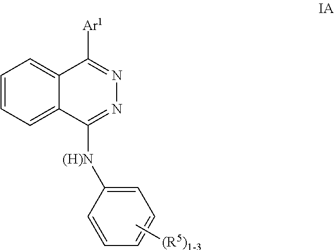

wherein Ar¹ is as defined for Formula I and each R⁵ is independently halo, —CN, —C(O)OR³, R⁶, —OR⁶, —N(R³)R⁶, alkyl optionally substituted with 1-3 halo, alkoxy optionally substituted with 1-3 halo, or heterocyclyl optionally substituted with R³, provided that the compound is not

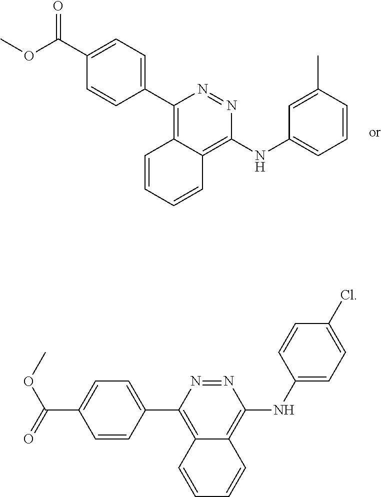 [SMILES: COC(=O)c1ccc(-c2nnc(Nc3cccc(C)c3)c3ccccc23)cc1 ; COC(=O)c1ccc(-c2nnc(Nc3ccc(Cl)cc3)c3ccccc23)cc1]

In certain embodiments, in the compound of Formula I or IA, each R⁵ is independently halo, —CN, —C(O)OR³, R⁶, —OR⁶, —N(R³)R⁶, (C₁-C₃)alkyl optionally substituted with 1-3 halo, alkoxy optionally substituted with 1-3 halo, or heterocyclyl optionally substituted with R³.

In certain embodiments, Ar² is

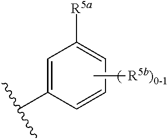

wherein R^{5a} is selected from halo, —CN, —C(O)OR³, R⁶, —OR⁶, —N(R³)R⁶, (C₁-C₃)alkyl optionally substituted with 1-3 halo, alkoxy optionally substituted with 1-3 halo, and heterocyclyl optionally substituted with R³; and R^{5b}, when present, is selected from halo, —CN, —C(O)OR³, R⁶, —OR⁶, —N(R³)R⁶, (C₁-C₃)alkyl optionally substituted with 1-3 halo, alkoxy optionally substituted with 1-3 halo, and heterocyclyl optionally substituted with R³.

In other embodiments, R^{5b}, when present, is selected from halo, —CN, —C(O)OR³, R⁶, —OR⁶, —N(R³)R⁶, (C₁-C₃)alkyl optionally substituted with 1-3 halo, and alkoxy.

In other embodiments, the JAK inhibitors of the present invention are of Formula IB:

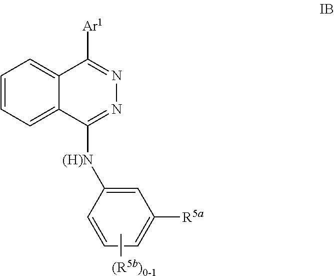

wherein Ar¹ is as defined for Formula I, R^{5a} and R^{5b} are as defined in paragraph [0012], and provided that the compound is not

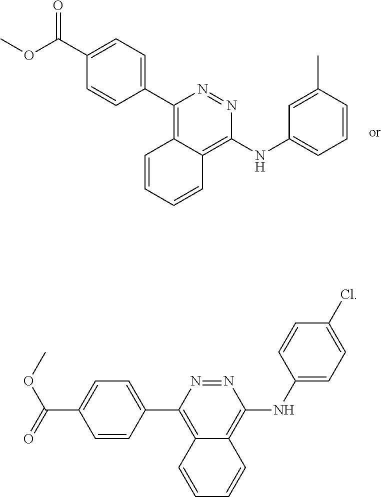 [SMILES: COC(=O)c1ccc(-c2nnc(Nc3cccc(C)c3)c3ccccc23)cc1 ; COC(=O)c1ccc(-c2nnc(Nc3ccc(Cl)cc3)c3ccccc23)cc1]

In other embodiments, Ar² is

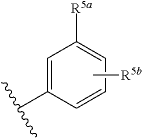

wherein R^{5a} is selected from halo, —CN, —C(O)OR³, R⁶, —OR⁶, —N(R³)R⁶, (C₁-C₃)alkyl optionally substituted with 1-3 halo, alkoxy optionally substituted with 1-3 halo, and heterocyclyl optionally substituted with R³; and R^{5b} is selected from halo, —CN, —C(O)OR³, R⁶, —OR⁶, —N(R³)R⁶, (C₁-C₃)alkyl optionally substituted with 1-3 halo, and alkoxy.

In certain embodiments, the JAK1 inhibitors of the present invention are of Formula IC:

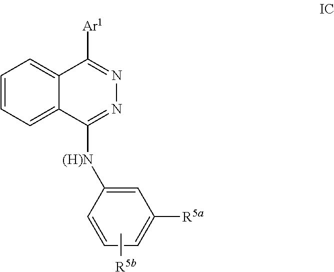

wherein Ar¹ is as defined for Formula I and wherein R^{5a} is selected from halo, —CN, —C(O)OR³, R⁶, —OR⁶, —N(R³)R⁶, (C₁-C₃)alkyl optionally substituted with 1-3 halo, alkoxy optionally substituted with 1-3 halo, and heterocyclyl optionally substituted with R³; and R^{5b} is selected from halo, —CN, —C(O)OR³, R⁶, —OR⁶, —N(R³)R⁶, (C₁-C₃)alkyl optionally substituted with 1-3 halo, and alkoxy.

In certain embodiments, the JAK1 inhibitors of the present invention are of Formula IC′:

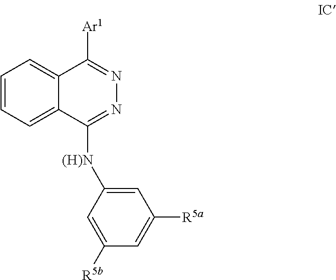

wherein Ar¹, R^{5a} and R^{5b} are as defined for Formula IC.

In certain embodiments, in the compound of Formula IC or IC′, R^{5a} and R^{5b} are the same substituent.

In certain embodiments, in the compound of Formula IC or IC′, R^{5a} and R^{5b} are different substituents.

In other embodiments, Ar² is

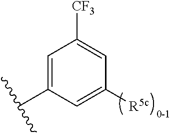

wherein R^{5c}, when present, is halo, alkyl, or —N(R³)R⁶.

In other embodiments, Ar² is selected from the group consisting of:

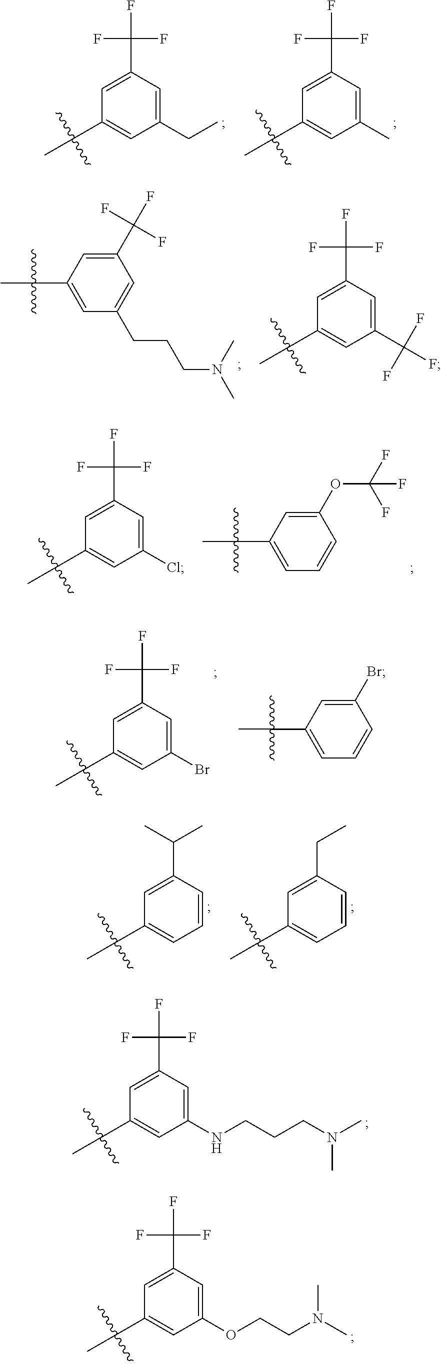

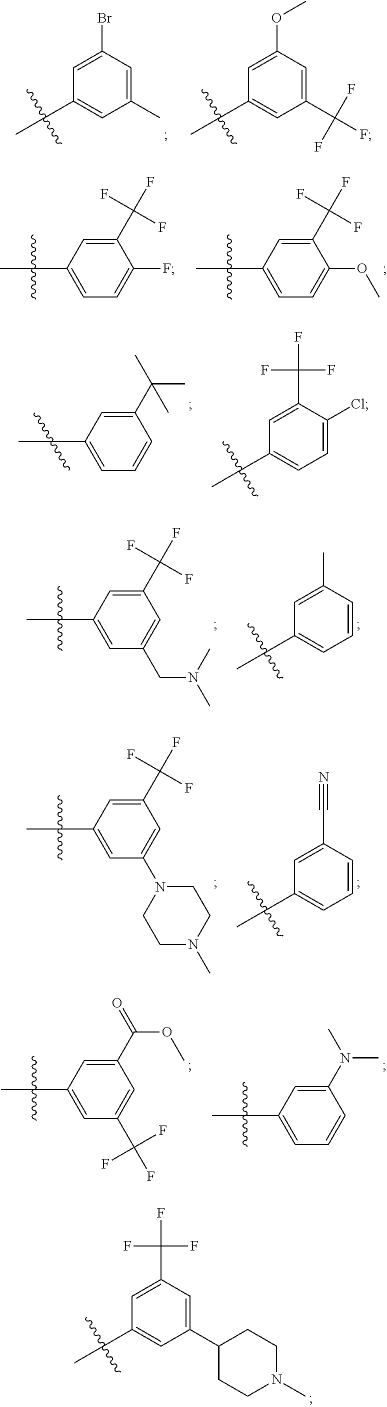

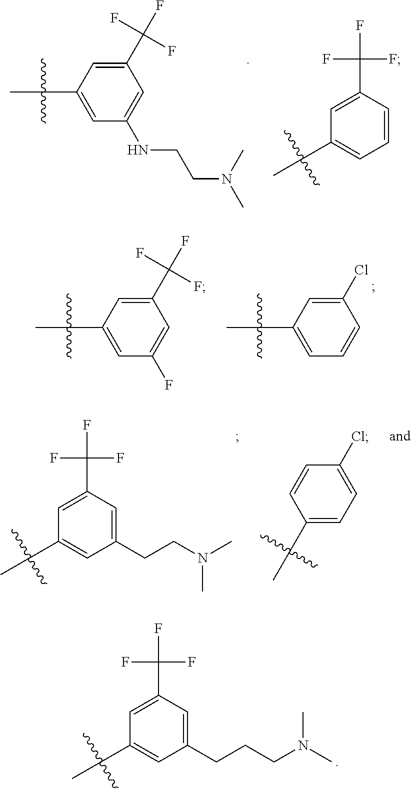

In other embodiments, R⁵, R^{5a}, R^{5b} are independently selected from the group consisting of:

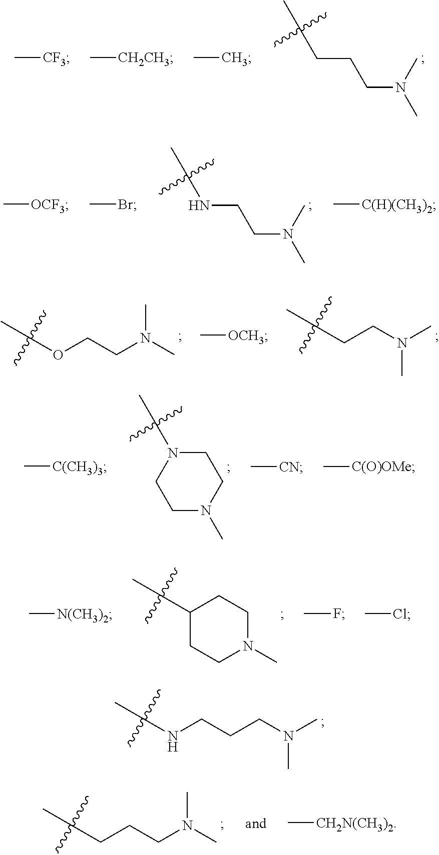

In other embodiments, the JAK1 inhibitors of the present invention are of Formula ID:

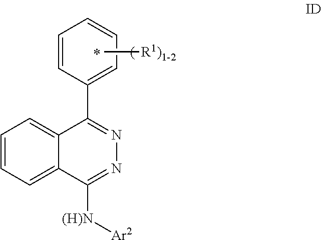

wherein Ar² is as defined for Formula I and each R¹ is independently —C(O)OR³, —C(O)R³, —C(O)N(H)alkylR⁴; —N(H)C(O)alkyl, —C(O)N(R³)(R⁴), —SO₂R³, heteroaryl optionally substituted with —NR³R², or heterocyclyl substituted with oxo; provided that the compound is not

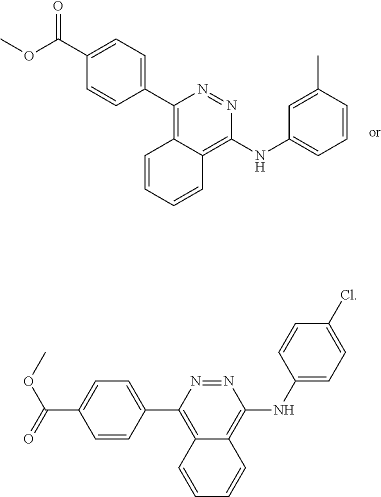 [SMILES: COC(=O)c1ccc(-c2nnc(Nc3cccc(C)c3)c3ccccc23)cc1 ; COC(=O)c1ccc(-c2nnc(Nc3ccc(Cl)cc3)c3ccccc23)cc1]

In certain embodiments of Formula I or ID, each R¹ is independently —C(O)OR³, heteroaryl optionally substituted with —NR³R², or heterocyclyl substituted with oxo.

In other embodiments, Ar¹ is

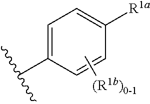

wherein R^{1a} is —C(O)OR³, —C(O)R³, —C(O)N(H)alkylR⁴; —N(H)C(O)alkyl, —C(O)N(R³)(R⁴), —SO₂R³, heteroaryl optionally substituted with —NR³R², or heterocyclyl substituted with oxo, and R^{1b}, when present, is —C(O)OR³, —C(O)R³, —C(O)N(H)alkylR⁴; —N(H)C(O)alkyl, C(O)N(R³)(R⁴), —SO₂R³, heteroaryl optionally substituted with —NR³R⁷, or heterocyclyl substituted with oxo.

In other embodiments, the JAK inhibitors of the present invention are of Formula IE:

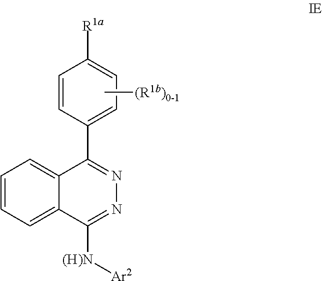

wherein Ar² is as defined for Formula I, R1a and R1B are as defined in paragraph [0026], and
provided that the compound is not

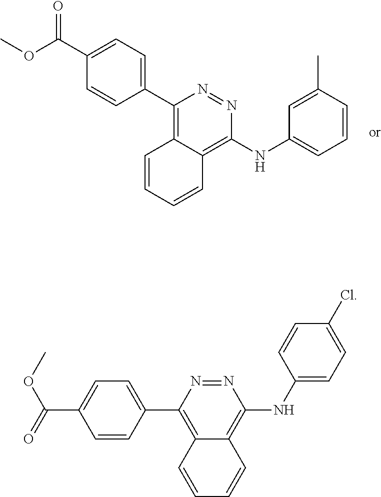 [SMILES: COC(=O)c1ccc(-c2nnc(Nc3cccc(C)c3)c3ccccc23)cc1 ; COC(=O)c1ccc(-c2nnc(Nc3ccc(Cl)cc3)c3ccccc23)cc1]

In certain embodiments, the JAK1 inhibitors of the present invention are of Formula IE′:

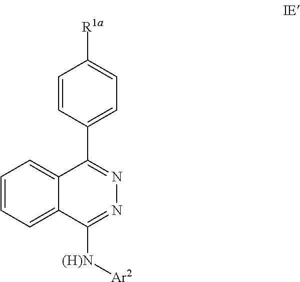

wherein Ar² and R^{1a} are as defined for Formula IE.

In other embodiments, the JAK1 inhibitors of the present invention are of Formula IE″:

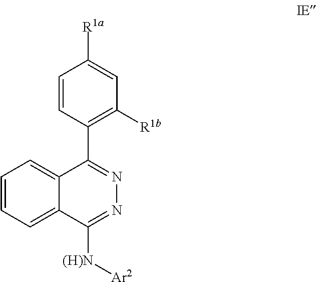

wherein Ar² and R^{1a} are as defined for Formula IE.

In other embodiments, Ar¹ is

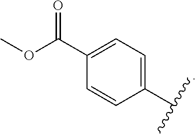 [SMILES: *c1ccc(C(=O)OC)cc1]

In other embodiments, Ar¹ of Formula I is heteroaryl optionally substituted with 1-2 R² groups, wherein the heteroaryl is 1H-indazolyl, pyrazolyl, benzotriazolyl, or benzofuranyl, isoindolyl.

In other embodiments, Ar¹ is selected from the group consisting of:

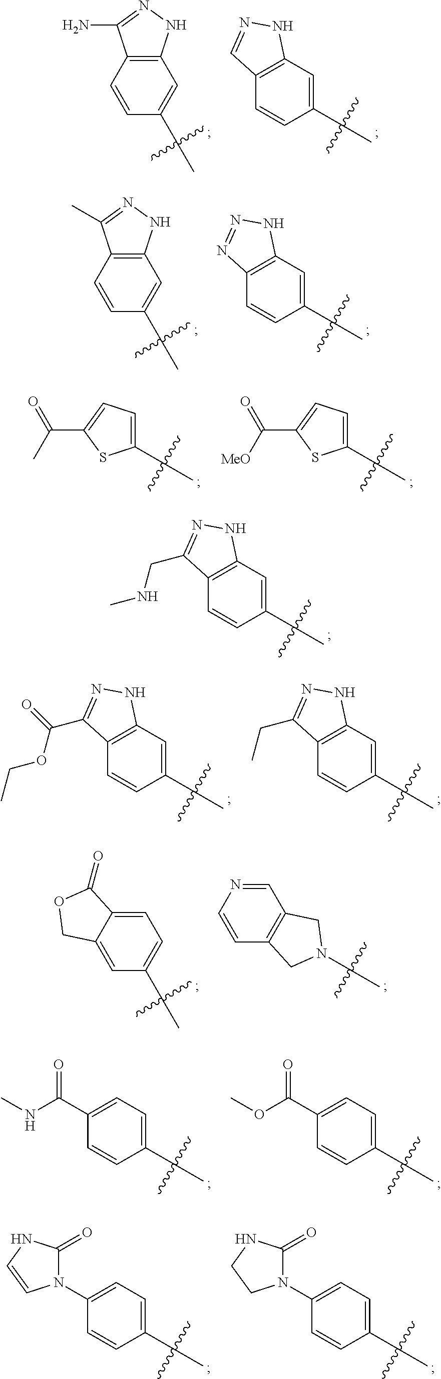

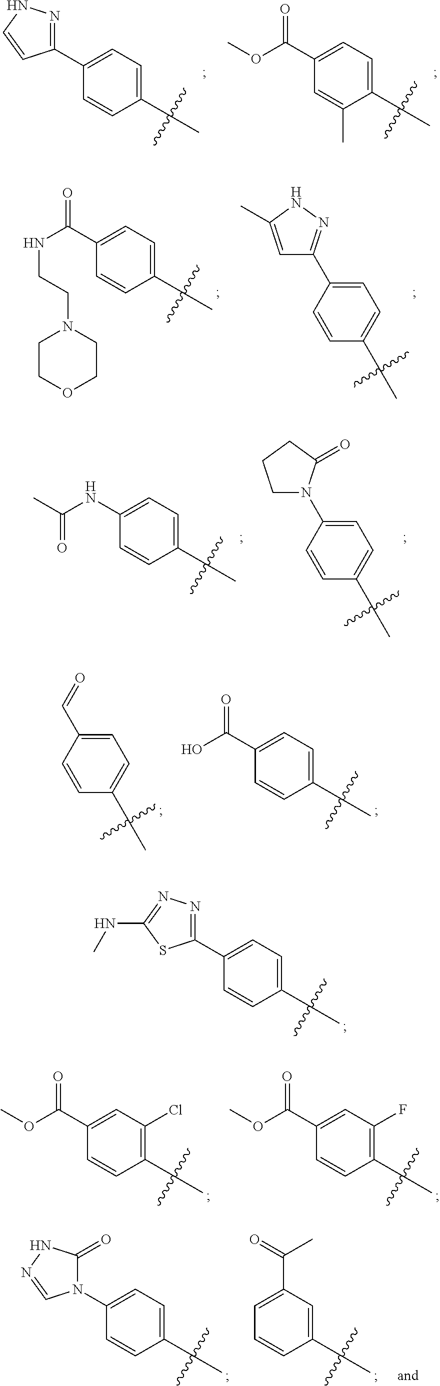

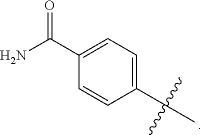 [SMILES: *c1ccc(C(N)=O)cc1]

In other embodiments, R¹, R^{1a}, R^{1b} are independently selected from the group consisting of:

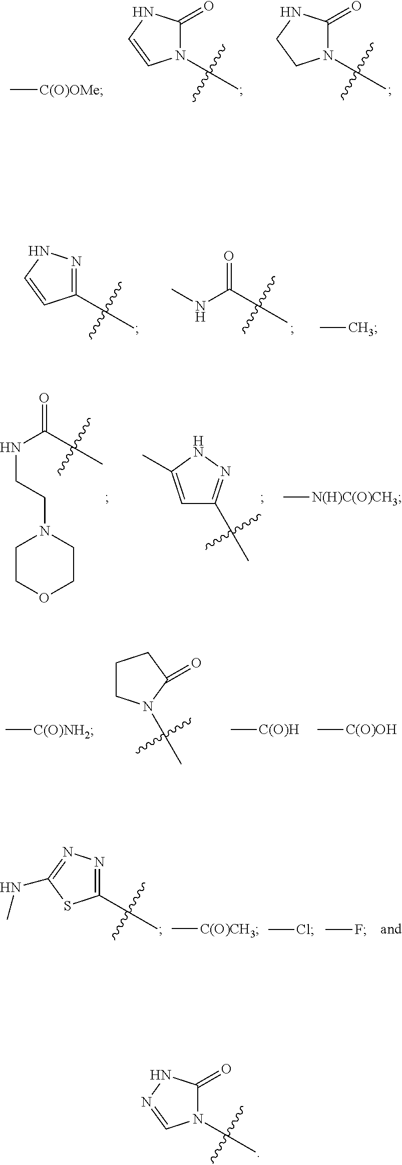

In other embodiments, R² is selected from the group consisting of:

| | | | | |
|---|---|---|---|---|
| —NH₂; | —CH₃; | —C(O)CH₃; | —C(O)OMe; | —C(O)OEt; |
| —CH₂N(H)CH₃; and | —CH₂CH₃. | | | |

In certain embodiments, R⁷ is H or (C₁-C₃)alkyl.

In another embodiment, the invention comprises a compound as shown in Table 1.

TABLE 1

| CMPD. | | |
|---|---|---|
| NO. | STRUCTURE | NAME |
| 1 | 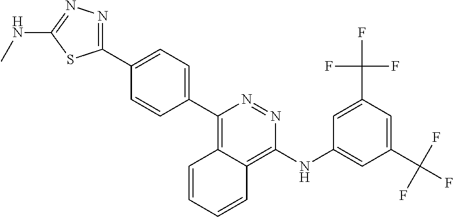 [SMILES: CNc1nnc(-c2ccc(-c3nnc(Nc4cc(C(F)(F)F)cc(C(F)(F)F)c4)c4ccccc34)cc2)s1] | N-[3,5-bis(trifluoromethyl)phenyl]-4-{4-[5- (methylamino)-1,3,4-thiadiazol-2- yl]phenyl}phthalazin-1-amine |
| 2 | 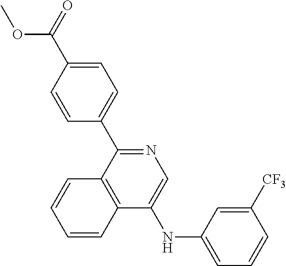 [SMILES: COC(=O)c1ccc(-c2ncc(Nc3cccc(C(F)(F)F)c3)c3ccccc23)cc1] | methyl 4-(4-{[3- (trifluoromethyl)phenyl]amino}isoquinolin-1- yl)benzoate |
| 3 | 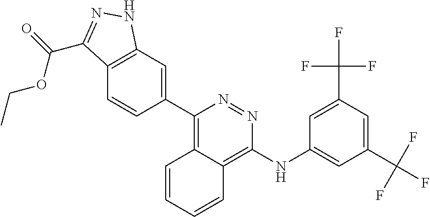 [SMILES: CCOC(=O)c1n[nH]c2cc(-c3nnc(Nc4cc(C(F)(F)F)cc(C(F)(F)F)c4)c4ccccc34)ccc12] | ethyl 6-(4-{[3,5- bis(trifluoromethyl)phenyl]amino}phthalazin-1- yl)-1H-indazole-3-carboxylate |
| 4 | 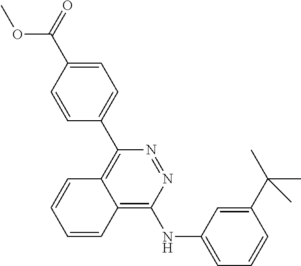 [SMILES: COC(=O)c1ccc(-c2nnc(Nc3cccc(C(C)(C)C)c3)c3ccccc23)cc1] | methyl 4-(4-{[3-(1,1- dimethylethyl)phenyl]amino}phthalazin-1- yl)benzoate |
| 5 | 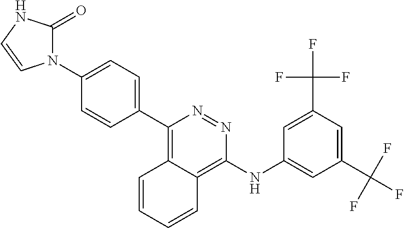 [SMILES: O=c1[nH]ccn1-c1ccc(-c2nnc(Nc3cc(C(F)(F)F)cc(C(F)(F)F)c3)c3ccccc23)cc1] | 1-[4-(4-{[3,5- bis(trifluoromethyl)phenyl]amino}phthalazin-1- yl)phenyl]-1,3-dihydro-2H-imidazol-2-one |
| 6 | 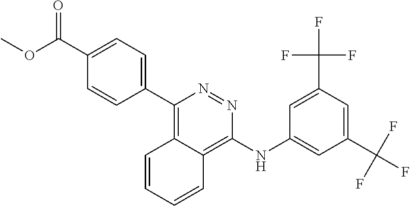 [SMILES: COC(=O)c1ccc(-c2nnc(Nc3cc(C(F)(F)F)cc(C(F)(F)F)c3)c3ccccc23)cc1] | methyl 4-(4-{[3,5- bis(trifluoromethyl)phenyl]amino}phthalazin-1- yl)-3-fluorobenzoate |
| 7 | 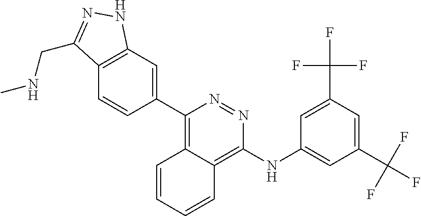 [SMILES: CNCc1n[nH]c2cc(-c3nnc(Nc4cc(C(F)(F)F)cc(C(F)(F)F)c4)c4ccccc34)ccc12] | N-[3,5-bis(trifluoromethyl)phenyl]-4-{3- [(methylamino)methyl]-1H-indazol-6- yl}phthalazin-1-amine |
| 8 | 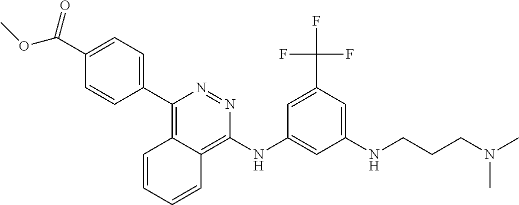 | methyl 4-(4-{[3-{[3- (dimethylamino)propyl]amino}-5- (trifluoromethyl)phenyl]amino}phthalazin-1- yl)benzoate |
| 9 | 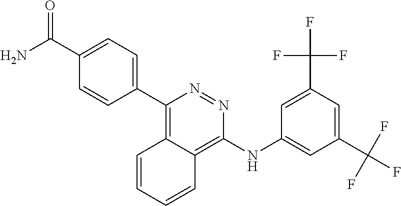 [SMILES: NC(=O)c1ccc(-c2nnc(Nc3cc(C(F)(F)F)cc(C(F)(F)F)c3)c3ccccc23)cc1] | 4-(4-{[3,5- bis(trifluoromethyl)phenyl]amino}phthalazin-1- yl)benzamide |
| 10 | 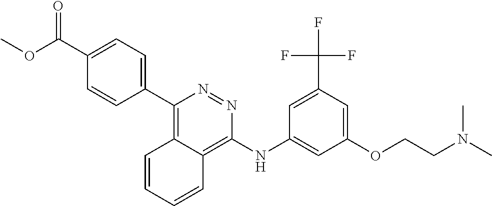 | methyl 4-(4-{[3-{[2- (dimethylamino)ethyl]oxy}-5- (trifluoromethyl)phenyl]amino}phthalazin-1- yl)benzoate |
| 11 | 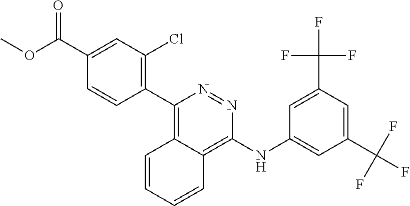 [SMILES: COC(=O)c1ccc(-c2nnc(Nc3cc(C(F)(F)F)cc(C(F)(F)F)c3)c3ccccc23)c(Cl)c1] | methyl 4-(4-{[3,5- bis(trifluoromethyl)phenyl]amino}phthalazin-1- yl)-3-chlorobenzoate |
| 12 | 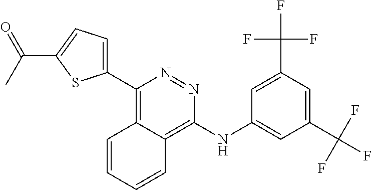 [SMILES: CC(=O)c1ccc(-c2nnc(Nc3cc(C(F)(F)F)cc(C(F)(F)F)c3)c3ccccc23)s1] | 1-[5-(4-{[3,5- bis(trifluoromethyl)phenyl]amino}phthalazin-1- yl)-2-thienyl]ethanone |
| 13 | 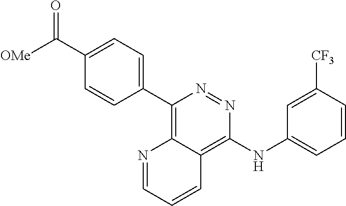 [SMILES: COC(=O)c1ccc(-c2nnc(Nc3cccc(C(F)(F)F)c3)c3cccnc23)cc1] | methyl 4-(5-{[3- (trifluoromethyl)phenyl]amino}pyrido[2,3- d]pyridazin-3-yl)benzoate |
| 14 | 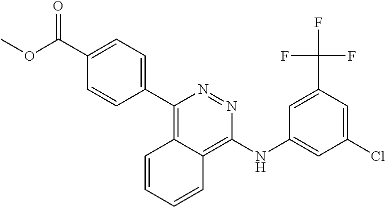 [SMILES: COC(=O)c1ccc(-c2nnc(Nc3cc(Cl)cc(C(F)(F)F)c3)c3ccccc23)cc1] | methyl 4-(4-{[3-chloro-5- (trifluoromethyl)phenyl]amino}phthalazin-1- yl)benzoate |
| 15 | 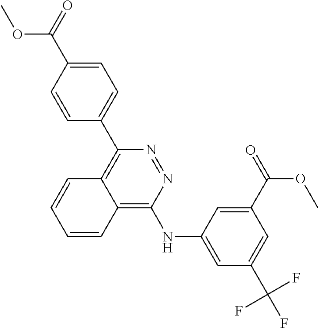 | methyl 3-[(4-{4- [(methyloxy)carbonyl]phenyl}phthalazin-1- yl)amino]-5-(trifluoromethyl)benzoate |
| 16 | 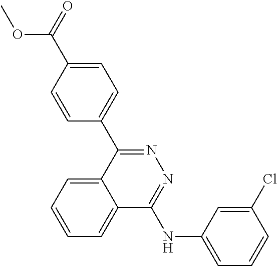 [SMILES: COC(=O)c1ccc(-c2nnc(Nc3cccc(Cl)c3)c3ccccc23)cc1] | methyl 4-{4-[(3- chlorophenyl)amino]phthalazin-1-yl}benzoate |
| 17 | 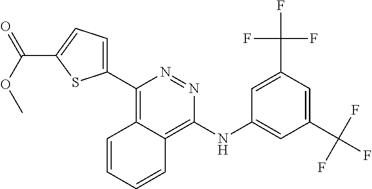 [SMILES: COC(=O)c1ccc(-c2nnc(Nc3cc(C(F)(F)F)cc(C(F)(F)F)c3)c3ccccc23)s1] | methyl 5-(4-{[3,5- bis(trifluoromethyl)phenyl]amino}phthalazin-1- yl)thiophene-2-carboxylate |
| 18 | 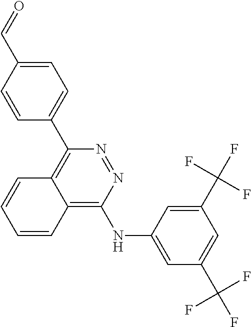 [SMILES: O=Cc1ccc(-c2nnc(Nc3cc(C(F)(F)F)cc(C(F)(F)F)c3)c3ccccc23)cc1] | 4-(4-{[3,5- bis(trifluoromethyl)phenyl]amino}phthalazin-1- yl)benzaldehyde |
| 19 | 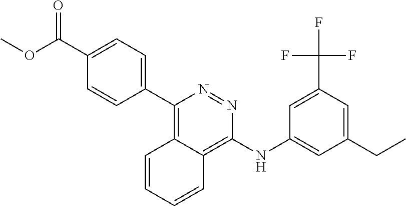 [SMILES: CCc1cc(Nc2nnc(-c3ccc(C(=O)OC)cc3)c3ccccc23)cc(C(F)(F)F)c1] | methyl 4-(4-{[3-ethyl-5- (trifluoromethyl)phenyl]amino}phthalazin-1- yl)benzoate |
| 20 | 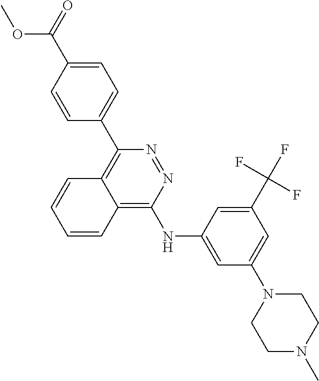 | methyl 4-(4-{[3-(4-methylpiperazin-1-yl)-5- (trifluoromethyl)phenyl]amino}phthalazin-1- yl)benzoate |
| 21 | 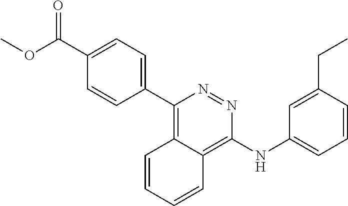 [SMILES: CCc1cccc(Nc2nnc(-c3ccc(C(=O)OC)cc3)c3ccccc23)c1] | methyl 4-{4-[(3-ethylphenyl)amino]phthalazin- 1-yl}benzoate |
| 22 | 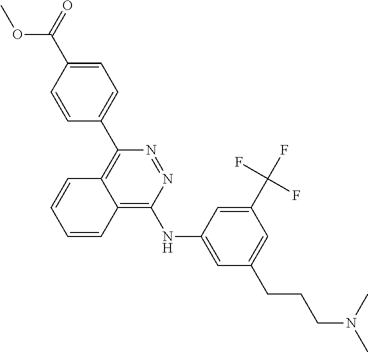 | methyl 4-[4-({3-[3-(dimethylamino)propyl]-5- (trifluoromethyl)phenyl}amino)phthalazin-1- yl]benzoate |
| 23 | 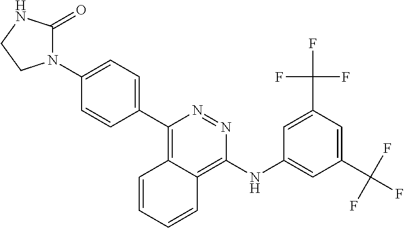 [SMILES: O=C1NCCN1c1ccc(-c2nnc(Nc3cc(C(F)(F)F)cc(C(F)(F)F)c3)c3ccccc23)cc1] | 1-[4-(4-{[3,5- bis(trifluoromethyl)phenyl]amino}phthalazin-1- yl)phenyl]imidazolidin-2-one |
| 24 | 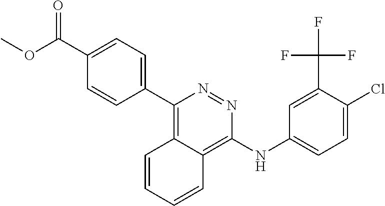 [SMILES: COC(=O)c1ccc(-c2nnc(Nc3ccc(Cl)c(C(F)(F)F)c3)c3ccccc23)cc1] | methyl 4-(4-{[4-chloro-3- (trifluoromethyl)phenyl]amino}phthalazin-1- yl)benzoate |
| 25 | 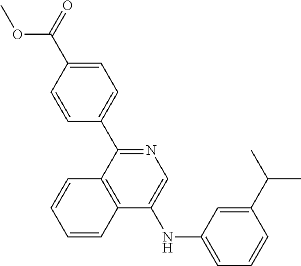 [SMILES: COC(=O)c1ccc(-c2ncc(Nc3cccc(C(C)C)c3)c3ccccc23)cc1] | methyl 4-(4-{[3-(1- methylethyl)phenyl]amino}isoquinolin-1- yl)benzoate |
| 27 | 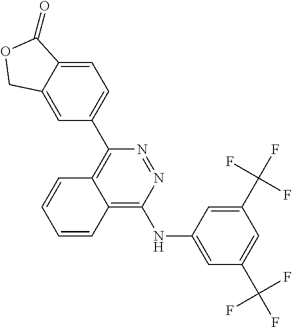 [SMILES: O=C1OCc2cc(-c3nnc(Nc4cc(C(F)(F)F)cc(C(F)(F)F)c4)c4ccccc34)ccc21] | 5-(4-{[3,5- bis(trifluoromethyl)phenyl]amino}phthalazin-1- yl)-2-benzofuran-1(3H)-one |
| 28 | 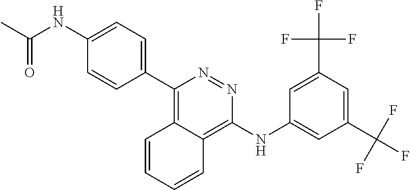 [SMILES: CC(=O)Nc1ccc(-c2nnc(Nc3cc(C(F)(F)F)cc(C(F)(F)F)c3)c3ccccc23)cc1] | N-[4-(4-{[3,5- bis(trifluoromethyl)phenyl]amino}phthalazin-1- yl)phenyl]acetamide |
| 29 | 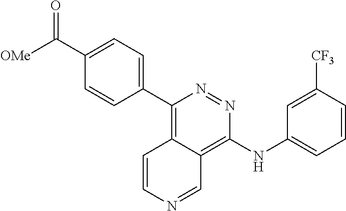 [SMILES: COC(=O)c1ccc(-c2nnc(Nc3cccc(C(F)(F)F)c3)c3cnccc23)cc1] | methyl 4-(4-{[3- (trifluoromethyl)phenyl]amino}pyrido[3,4- d]pyridazin-1-yl)benzoate |
| 30 | 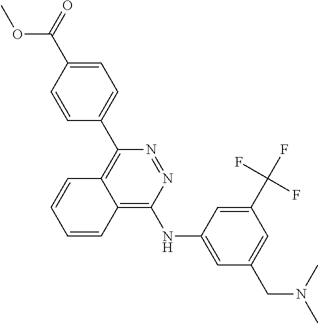 | methyl 4-[4-({3-[(dimethylamino)methyl]-5- (trifluoromethyl)phenyl}amino)phthalazin-1- yl]benzoate |
| 31 | 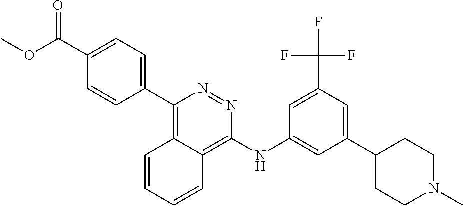 | methyl 4-(4-{[3-(1-methylpiperidin-4-yl)-5- (trifluoromethyl)phenyl]amino}phthalazin-1- yl)benzoate |
| 32 | 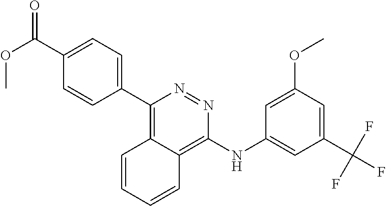 [SMILES: COC(=O)c1ccc(-c2nnc(Nc3cc(OC)cc(C(F)(F)F)c3)c3ccccc23)cc1] | methyl 4-(4-{[3-(methyloxy)-5- (trifluoromethyl)phenyl]amino}phthalazin-1- yl)benzoate |
| 33 | 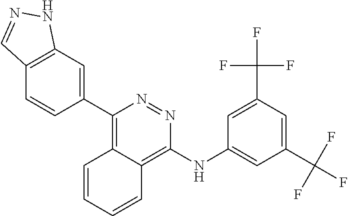 [SMILES: FC(F)(F)c1cc(Nc2nnc(-c3ccc4cn[nH]c4c3)c3ccccc23)cc(C(F)(F)F)c1] | N-[3,5-bis(trifluoromethyl)phenyl]-4-(1H- indazol-6-yl)phthalazin-1-amine |
| 34 | 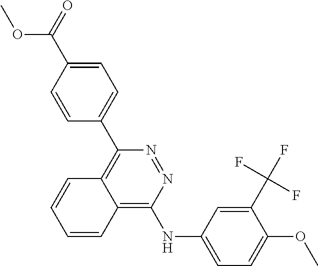 | methyl 4-(4-{[4-(methyloxy)-3- (trifluoromethyl)phenyl]amino}phthalazin-1- yl)benzoate |
| 35 | 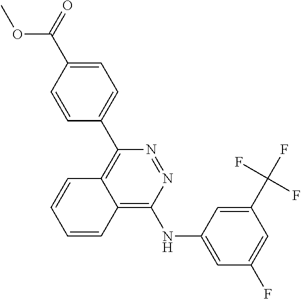 [SMILES: COC(=O)c1ccc(-c2nnc(Nc3cc(F)cc(C(F)(F)F)c3)c3ccccc23)cc1] | methyl 4-(4-{[3-fluoro-5- (trifluoromethyl)phenyl]amino}phthalazin-1- yl)benzoate |
| 36 | 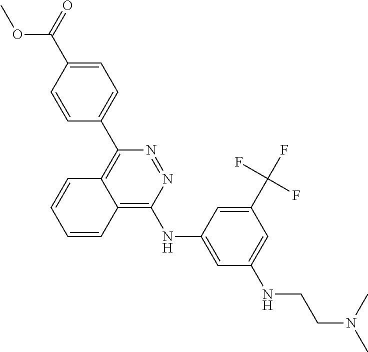 | methyl 4-(4-{[3-{[2- (dimethylamino)ethyl]amino}-5- (trifluoromethyl)phenyl]amino}phthalazin-1- yl)benzoate |
| 37 | 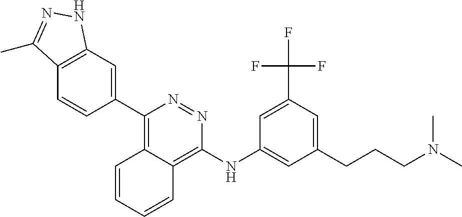 | N-{3-[3-(dimethylamino)propyl]-5- (trifluoromethyl)phenyl}-4-(3-methyl-1H- indazol-6-yl)phthalazin-1-amine |
| 38 | 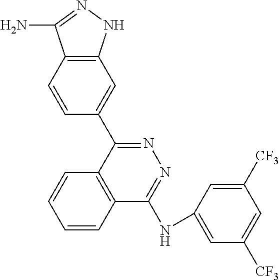 [SMILES: Nc1n[nH]c2cc(-c3nnc(Nc4cc(C(F)(F)F)cc(C(F)(F)F)c4)c4ccccc34)ccc12] | 4-(3-amino-1H-indazol-6-yl)-N-[3,5- bis(trifluoromethyl)phenyl]phthalazin-1-amine |
| 39 | 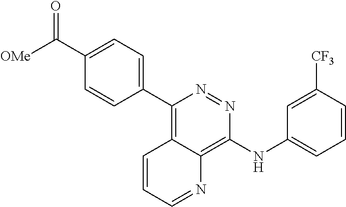 [SMILES: COC(=O)c1ccc(-c2nnc(Nc3cccc(C(F)(F)F)c3)c3ncccc23)cc1] | methyl 4-(8-{[3- (trifluoromethyl)phenyl]amino}pyrido[2,3- d]pyridazin-5-yl)benzoate |
| 40 | 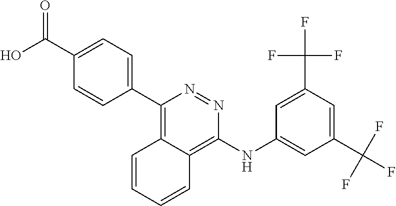 [SMILES: O=C(O)c1ccc(-c2nnc(Nc3cc(C(F)(F)F)cc(C(F)(F)F)c3)c3ccccc23)cc1] | 4-(4-{[3,5- bis(trifluoromethyl)phenyl]amino}phthalazin-1- yl)benzoic acid |
| 41 | 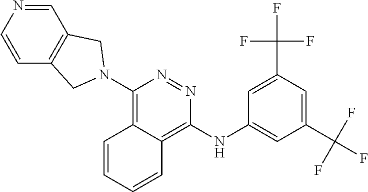 [SMILES: FC(F)(F)c1cc(Nc2nnc(N3Cc4ccncc4C3)c3ccccc23)cc(C(F)(F)F)c1] | N-[3,5-bis(trifluoromethyl)phenyl]-4-(1,3- dihydro-2H-pyrrolo[3,4-c]pyridin-2- yl)phthalazin-1-amine |
| 42 | 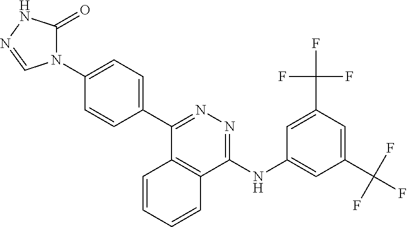 [SMILES: O=c1[nH]ncn1-c1ccc(-c2nnc(Nc3cc(C(F)(F)F)cc(C(F)(F)F)c3)c3ccccc23)cc1] | 4-[4-(4-{[3,5- bis(trifluoromethyl)phenyl]amino}phthalazin-1- yl)phenyl]-2,4-dihydro-3H-1,2,4-triazol-3-one |
| 43 | 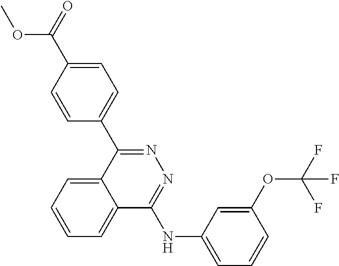 [SMILES: COC(=O)c1ccc(-c2nnc(Nc3cccc(OC(F)(F)F)c3)c3ccccc23)cc1] | methyl 4-[4-({3- [(trifluoromethyl)oxy]phenyl}amino)phthalazin- 1-yl]benzoate |
| 44 | 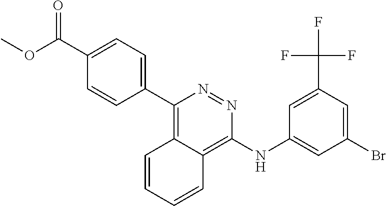 [SMILES: COC(=O)c1ccc(-c2nnc(Nc3cc(Br)cc(C(F)(F)F)c3)c3ccccc23)cc1] | methyl 4(4-{[3-bromo-5- (trifluoromethyl)phenyl]amino}phthalazin-1- yl)benzoate |
| 45 | 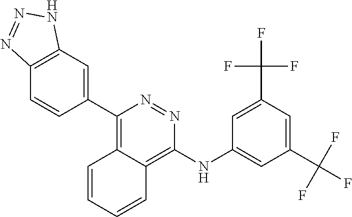 [SMILES: FC(F)(F)c1cc(Nc2nnc(-c3ccc4nn[nH]c4c3)c3ccccc23)cc(C(F)(F)F)c1] | 4-(1H-1,2,3-benzotriazol-6-yl)-N-[3,5- bis(trifluoromethyl)phenyl]phthalazin-1-amine |
| 46 | 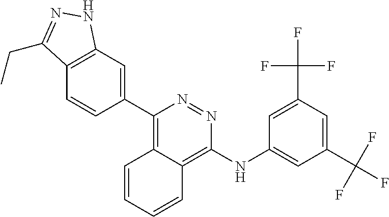 [SMILES: CCc1n[nH]c2cc(-c3nnc(Nc4cc(C(F)(F)F)cc(C(F)(F)F)c4)c4ccccc34)ccc12] | N-[3,5-bis(trifluoromethyl)phenyl]-4-(3-ethyl- 1H-indazol-6-yl)phthalazin-1-amine |
| 48 | 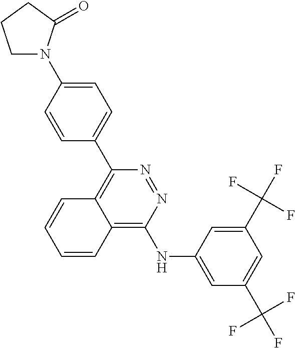 [SMILES: O=C1CCCN1c1ccc(-c2nnc(Nc3cc(C(F)(F)F)cc(C(F)(F)F)c3)c3ccccc23)cc1] | 1-[4-(4-{[3,5- bis(trifluoromethyl)phenyl]amino}phthalazin-1- yl)phenyl]pyrrolidin-2-one |
| 49 | 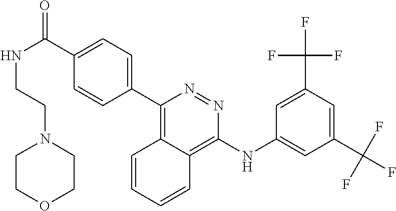 [SMILES: O=C(NCCN1CCOCC1)c1ccc(-c2nnc(Nc3cc(C(F)(F)F)cc(C(F)(F)F)c3)c3ccccc23)cc1] | 4-(4-{[3,5- bis(trifluoromethyl)phenyl]amino}phthalazin-1- yl)-N-(2-morpholin-4-ylethyl)benzamide |
| 50 | 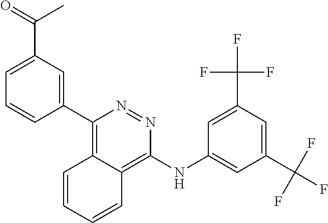 [SMILES: CC(=O)c1cccc(-c2nnc(Nc3cc(C(F)(F)F)cc(C(F)(F)F)c3)c3ccccc23)c1] | 1-[3-(4-{[3,5- bis(trifluoromethyl)phenyl]amino}phthalazin-1- yl)phenyl]ethanone |
| 51 | 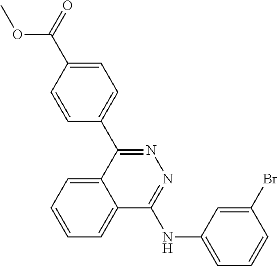 [SMILES: COC(=O)c1ccc(-c2nnc(Nc3cccc(Br)c3)c3ccccc23)cc1] | methyl 4-{4-[(3- bromophenyl)amino]phthalazin-1-yl}benzoate |
| 52 | 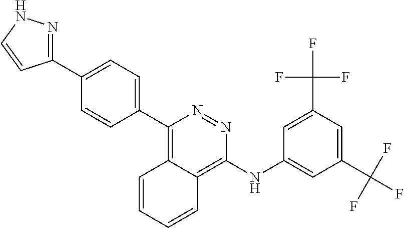 [SMILES: FC(F)(F)c1cc(Nc2nnc(-c3ccc(-c4cc[nH]n4)cc3)c3ccccc23)cc(C(F)(F)F)c1] | N-[3,5-bis(trifluoromethyl)phenyl]-4-[4-(1H- pyrazol-3-yl)phenyl]phthalazin-1-amine |
| 53 | 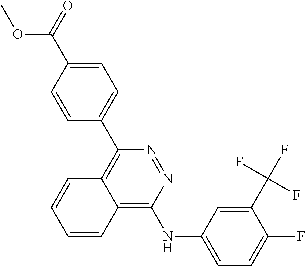 [SMILES: COC(=O)c1ccc(-c2nnc(Nc3ccc(F)c(C(F)(F)F)c3)c3ccccc23)cc1] | methyl 4-(4-{[4-fluoro-3- (trifluoromethyl)phenyl]amino}phthalazin-1- yl)benzoate |
| 54 | 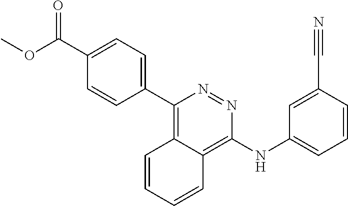 [SMILES: COC(=O)c1ccc(-c2nnc(Nc3cccc(C#N)c3)c3ccccc23)cc1] | methyl 4-{4-[(3-cyanophenyl)amino]phthalazin- 1-yl}benzoate |
| 55 | 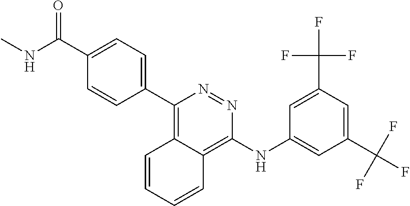 [SMILES: CNC(=O)c1ccc(-c2nnc(Nc3cc(C(F)(F)F)cc(C(F)(F)F)c3)c3ccccc23)cc1] | 4-(4-{[3,5- bis(trifluoromethyl)phenyl]amino}phthalazin-1- yl)-N-methylbenzamide |
| 56 | 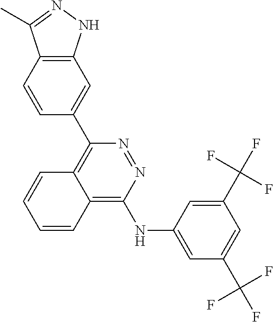 [SMILES: Cc1n[nH]c2cc(-c3nnc(Nc4cc(C(F)(F)F)cc(C(F)(F)F)c4)c4ccccc34)ccc12] | N-[3,5-bis(trifluoromethyl)phenyl]-4-(3-methyl- 1H-indazol-6-yl)phthalazin-1-amine |
| 57 | 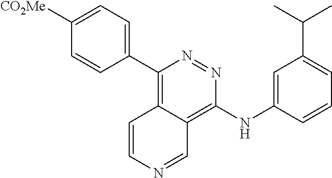 [SMILES: COC(=O)c1ccc(-c2nnc(Nc3cccc(C(C)C)c3)c3cnccc23)cc1] | methyl 4-(4-{[3-(1- methylethyl)phenyl]amino}pyrido[3,4- d]pyridazin-1-yl)benzoate |
| 58 | 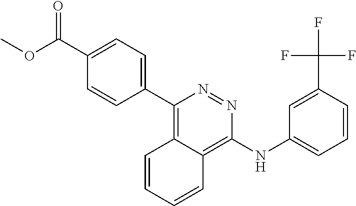 [SMILES: COC(=O)c1ccc(-c2nnc(Nc3cccc(C(F)(F)F)c3)c3ccccc23)cc1] | methyl 4-(4-{[3- (trifluoromethyl)phenyl]amino}phthalazin-1- yl)benzoate |
| 59 | 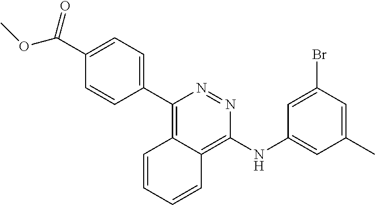 | methyl 4-{4-[(3-bromo-5- methylphenyl)amino]phthalazin-1-yl}benzoate |
| 60 | 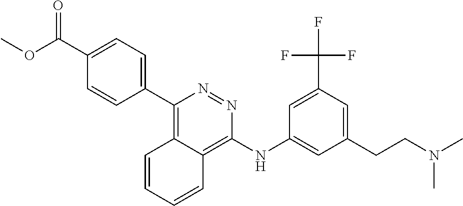 | methyl 4-[4-({3-[2-(dimethylamino)ethyl]-5- (trifluoromethyl)phenyl}amino)phthalazin-1- yl]benzoate |
| 61 | 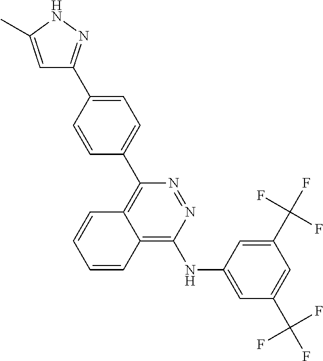 [SMILES: Cc1cc(-c2ccc(-c3nnc(Nc4cc(C(F)(F)F)cc(C(F)(F)F)c4)c4ccccc34)cc2)n[nH]1] | N-[3,5-bis(trifluoromethyl)phenyl]-4-[4-(5- methyl-1H-pyrazol-3-yl)phenyl]phthalazin-1- amine |
| 62 | 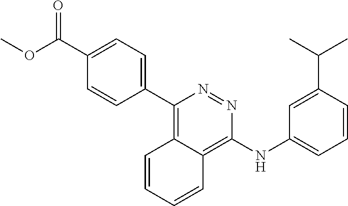 [SMILES: COC(=O)c1ccc(-c2nnc(Nc3cccc(C(C)C)c3)c3ccccc23)cc1] | methyl 4-(4-{[3-(1- methylethyl)phenyl]amino}phthalazin-1- yl)benzoate |
| 63 | 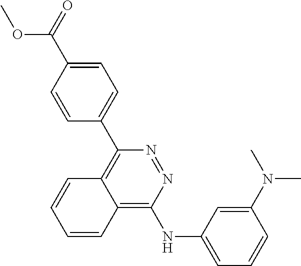 | methyl 4-(4-{[3- (dimethylamino)phenyl]amino}phthalazin-1- yl)benzoate |
| 64 | 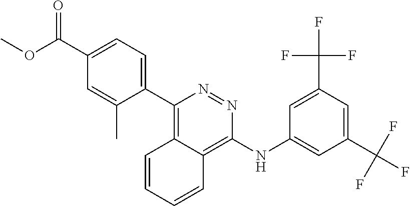 [SMILES: COC(=O)c1ccc(-c2nnc(Nc3cc(C(F)(F)F)cc(C(F)(F)F)c3)c3ccccc23)c(C)c1] | methyl 4-(4-{[3,5- bis(trifluoromethyl)phenyl]amino}phthalazin-1- yl)-3-methylbenzoate |
| 65 | 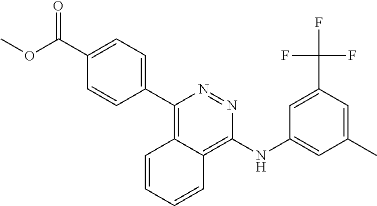 | methyl 4-(4-{[3-methyl-5- (trifluoromethyl)phenyl]amino}phthalazin-1- yl)benzoate |
| 66 | 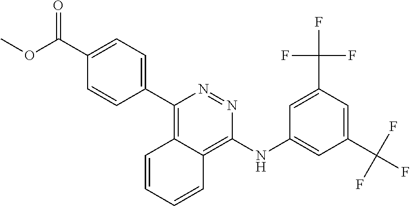 [SMILES: COC(=O)c1ccc(-c2nnc(Nc3cc(C(F)(F)F)cc(C(F)(F)F)c3)c3ccccc23)cc1] | methyl 4-(4-{[3,5- bis(trifluoromethyl)phenyl]amino}phthalazin-1- yl)benzoate |

The invention also comprises as another embodiment, a composition comprising a JAK1 inhibitor compound according to any one of the preceding embodiments together with a pharmaceutically acceptable diluent, excipient, and/or carrier. Such compositions are substantially free of non-pharmaceutically acceptable components, i.e., contain amounts of non-pharmaceutically acceptable components lower than permitted by US regulatory requirements at the time of filing this application. In some embodiments of this aspect, if the compound is dissolved or suspended in water, the composition further optionally comprises an additional pharmaceutically acceptable carrier, diluent, or excipient.

The invention also comprises as another embodiment a method for inhibiting JAK1 comprising administering a compound of Formula I:

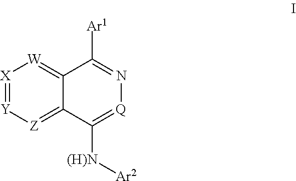

or a pharmaceutically acceptable salt thereof, wherein:

Ar¹ is phenyl optionally substituted with 1-2 R¹ groups or optionally fused to a 5-6 membered heterocyclyl, heterocyclyl, or heteroaryl optionally substituted with 1-2 R² groups;

Ar² is phenyl optionally substituted with 1-3 R⁵ groups;

each R¹ is independently halo, alkyl, —C(O)OR³, —C(O)R³, —C(O)N(H)alkylR⁴, —N(H)C(O)alkyl, —C(O)N(R³)(R⁴), —SO₂R³, heteroaryl optionally substituted with R³ or —NR³R⁷, or heterocyclyl substituted with oxo;

each R² is independently —N(R³)(R⁴), -alkylN(R³)(R⁴), oxo, alkyl, —C(O)R³, or —C(O)OR³;

R³ is H or alkyl;

R⁴ is H or alkyl optionally substituted with heterocyclyl;

each R⁵ is independently halo, —CN, —C(O)OR³, R⁶, —OR⁶, —N(R³)R⁶, alkyl optionally substituted with 1-3 halo, alkoxy optionally substituted with 1-3 halo, or heterocyclyl optionally substituted with R³;

R⁶ is alkyl optionally substituted with —NR³R⁷;

R⁷ is H or alkyl;

Q is C(H) or N;

W is C(H) or N;

X is C(H) or N;

Y is C(H) or N; and

Z is C(H) or N.

The invention comprises as a further embodiment a method for treating a disease JAK1 mediates or is implicated in a subject in need thereof comprising administrating to the subject a therapeutically effective amount of a JAK1 inhibitor compound according to any one of the preceding embodiments, or a composition comprising a JAK1 inhibitor according to any one of the preceding embodiments together with a pharmaceutically acceptable diluent, excipient, and/or carrier. The diseases JAK1 mediates or is implicated in that may be treated includes, without limitation, cancer, inflammatory disorders, and autoimmune diseases.

The invention also comprises as another embodiment a method for treating cancer in a subject in need of such treatment comprising administering to the subject an effective amount of a JAK1 inhibitor compound or a pharmaceutical composition according to any one of the preceding embodiments.

The cancers to be treated include, but are not limited to, carcinomas, sarcomas, lymphomas, leukemias, myelomas, germ cell tumors, blastomas, tumors of the central and peripheral nervous system and other tumors including melanomas, seminoma and Kaposi's sarcoma and the like.

The invention also comprises as another embodiment a method for treating inflammatory disorders in a subject in need of such treatment comprising administering to the subject an effective amount of a JAK1 inhibitor compound or a pharmaceutical composition according to any one of the preceding embodiments.

The invention also comprises as another embodiment a method for treating autoimmune diseases in a subject in need of such treatment comprising administering to the subject an effective amount of a JAK1 inhibitor compound or a pharmaceutical composition according to any one of the preceding embodiments.

The immune and/or inflammatory disorders to be treated include, but are not limited to, acquired immunodeficiency syndrome (AIDS), Addison's disease, adult respiratory distress syndrome, allergies, ankylosing spondylitis, amyloidosis, asthma, autoimmune hemolytic anemia, autoimmune thyroiditis, Crohn's disease, episodic lymphopenia with lymphocytotoxins, erythroblastosis fetalis, Goodpasture's syndrome, Graves' disease, Hashimoto's thyroiditis, hypereosinophilia, irritable bowel syndrome and other interbowel diseases, Lupus, myasthenia gravis, myocardial or pericardial inflammation, pancreatitis, polymyositis, psoriasis, Reiter's syndrome, scleroderma, systemic analphylaxis, lucerative colitis, nephritis (including glomerulonephritis), gout, arthritis (such as rheumatoid arthritis and osteoarthritis), erythema, dermatitis, dermatomyositis, bronchitis, cholecystitis, and gastritis.

The invention also comprises as another embodiment the use of a JAK1 inhibitor compound according to any one of the preceding embodiments for the preparation of a medicament for treating cancer.

The invention also comprises as another embodiment the use of a JAK1 inhibitor compound according to any one of the preceding embodiments for the preparation of a medicament for treating inflammatory disorders.

The invention also comprises as another embodiment the use of a JAK1 inhibitor compound according to any one of the preceding embodiments for the preparation of a medicament for treating autoimmune diseases.

It is to be understood throughout this specification that any and all possible combinations of the specific embodiments as recited hereinabove fall within the scope of the invention, and that the recitation of particular embodiments is not intended to be exclusive of others. In non-limiting examples, compounds of Formula I in which R⁵ is defined in accordance with the embodiment disclosed in paragraph [0011] and in which R⁷ is defined in accordance with the embodiment disclosed in paragraph [0035] fall within the scope of the invention, as do compounds of Formula I in which Ar¹ is defined in accordance with the embodiment disclosed in paragraph [0030] and in which Ar² is defined in accordance with the embodiment disclosed in paragraph [0021]. It is to be further understood that any embodiment of this specification which includes a variable that is not defined, that the definition of that variable can be as defined in any of the embodiments disclosed herein wherein this variable in question is defined.

### Pharmaceutical Formulations and Dosage Forms

Administration of the compounds of this disclosure, or their pharmaceutically acceptable salts, in pure form or in an appropriate pharmaceutical composition, can be carried out via any of the accepted modes of administration or agents for serving similar utilities. Thus, administration can be, for example, orally, nasally, parenterally (intravenous, intramuscular, or subcutaneous), topically, transdermally, intravaginally, intravesically, intracistemally, or rectally, in the form of solid, semi-solid, lyophilized powder, or liquid dosage forms, such as for example, tablets, suppositories, pills, soft elastic and hard gelatin capsules, powders, solutions, suspensions, or aerosols, or the like, preferably in unit dosage forms suitable for simple administration of precise dosages.

The compositions will include a conventional pharmaceutical carrier, excipient, and/or diluent and a compound of this disclosure as the/an active agent, and, in addition, can include carriers and adjuvants, etc.

Adjuvants include preserving, wetting, suspending, sweetening, flavoring, perfuming, emulsifying, and dispensing agents. Prevention of the action of microorganisms can be ensured by various antibacterial and antifungal agents, for example, parabens, chlorobutanol, phenol, sorbic acid, and the like. It can also be desirable to include isotonic agents, for example sugars, sodium chloride, and the like. Prolonged absorption of the injectable pharmaceutical form can be brought about by the use of agents delaying absorption, for example, aluminum monostearate and gelatin.

If desired, a pharmaceutical composition of the compounds in this disclosure can also contain minor amounts of auxiliary substances such as wetting or emulsifying agents, pH buffering agents, antioxidants, and the like, such as, for example, citric acid, sorbitan monolaurate, triethanolamine oleate, butylalted hydroxytoluene, etc.

The choice of formulation depends on various factors such as the mode of drug administration (e.g., for oral administration, formulations in the form of tablets, pills or capsules are preferred) and the bioavailability of the drug substance. Recently, pharmaceutical formulations have been developed especially for drugs that show poor bioavailability based upon the principle that bioavailability can be increased by increasing the surface area i.e., decreasing particle size. For example, U.S. Pat. No. 4,107,288 describes a pharmaceutical formulation having particles in the size range from 10 to 1,000 nm in which the active material is supported on a crosslinked matrix of macromolecules. U.S. Pat. No. 5,145,684 describes the production of a pharmaceutical formulation in which the drug substance is pulverized to nanoparticles (average particle size of 400 nm) in the presence of a surface modifier and then dispersed in a liquid medium to give a pharmaceutical formulation that exhibits remarkably high bioavailability.

Compositions suitable for parenteral injection can comprise physiologically acceptable sterile aqueous or nonaqueous solutions, dispersions, suspensions or emulsions, and sterile powders for reconstitution into sterile injectable solutions or dispersions. Examples of suitable aqueous and nonaqueous carriers, diluents, solvents or vehicles include water, ethanol, polyols (propyleneglycol, polyethyleneglycol, glycerol, and the like), suitable mixtures thereof, vegetable oils (such as olive oil) and injectable organic esters such as ethyl oleate. Proper fluidity can be maintained, for example, by the use of a coating such as lecithin, by the maintenance of the required particle size in the case of dispersions and by the use of surfactants.

One preferable route of administration is oral, using a convenient daily dosage regimen that can be adjusted according to the degree of severity of the disease-state to be treated.

Solid dosage forms for oral administration include capsules, tablets, pills, powders, and granules. In such solid dosage forms, the active compound is admixed with at least one inert customary excipient (or carrier) such as sodium citrate or dicalcium phosphate or (a) fillers or extenders, as for example, starches, lactose, sucrose, glucose, mannitol, and silicic acid, (b) binders, as for example, cellulose derivatives, starch, alignates, gelatin, polyvinylpyrrolidone, sucrose, and gum acacia, (c) humectants, as for example, glycerol, (d) disintegrating agents, as for example, agar-agar, calcium carbonate, potato or tapioca starch, alginic acid, croscarmellose sodium, complex silicates, and sodium carbonate, (e) solution retarders, as for example paraffin, (f) absorption accelerators, as for example, quaternary ammonium compounds, (g) wetting agents, as for example, cetyl alcohol, and glycerol monostearate, magnesium stearate and the like (h) adsorbents, as for example, kaolin and bentonite, and (i) lubricants, as for example, talc, calcium stearate, magnesium stearate, solid polyethylene glycols, sodium lauryl sulfate, or mixtures thereof. In the case of capsules, tablets, and pills, the dosage forms can also comprise buffering agents.

Solid dosage forms, as described above, can be prepared with coatings and shells, such as enteric coatings and others well known in the art. They can contain pacifying agents, and can also be of such composition that they release the active compound or compounds in a certain part of the intestinal tract in a delayed manner. Examples of embedded compositions that can be used are polymeric substances and waxes. The active compounds can also be in microencapsulated form, if appropriate, with one or more of the above-mentioned excipients.

Liquid dosage forms for oral administration include pharmaceutically acceptable emulsions, solutions, suspensions, syrups, and elixirs. Such dosage forms are prepared, for example, by dissolving, dispersing, etc., a compound(s) of this disclosure, or a pharmaceutically acceptable salt thereof, and optional pharmaceutical adjuvants in a carrier, such as, for example, water, saline, aqueous dextrose, glycerol, ethanol and the like; solubilizing agents and emulsifiers, as for example, ethyl alcohol, isopropyl alcohol, ethyl carbonate, ethyl acetate, benzyl alcohol, benzyl benzoate, propyleneglycol, 1,3-butyleneglycol, dimethylformamide; oils, in particular, cottonseed oil, groundnut oil, corn germ oil, olive oil, castor oil and sesame oil, glycerol, tetrahydrofurfuryl alcohol, polyethyleneglycols and fatty acid esters of sorbitan; or mixtures of these substances, and the like, to thereby form a solution or suspension.

Suspensions, in addition to the active compounds, can contain suspending agents, as for example, ethoxylated isostearyl alcohols, polyoxyethylene sorbitol and sorbitan esters, microcrystalline cellulose, aluminum metahydroxide, bentonite, agar-agar and tragacanth, or mixtures of these substances, and the like.

Compositions for rectal administrations are, for example, suppositories that can be prepared by mixing the compounds of this disclosure with, for example, suitable non-irritating excipients or carriers such as cocoa butter, polyethyleneglycol or a suppository wax, which are solid at ordinary temperatures but liquid at body temperature and therefore, melt while in a suitable body cavity and release the active component therein.

Dosage forms for topical administration of a compound of this disclosure include ointments, powders, sprays, and inhalants. The active component is admixed under sterile conditions with a physiologically acceptable carrier and any preservatives, buffers, or propellants as can be required. Ophthalmic formulations, eye ointments, powders, and solutions are also contemplated for the compounds in this disclosure.

Compressed gases can be used to disperse a compound of this disclosure in aerosol form. Inert gases suitable for this purpose are nitrogen, carbon dioxide, etc.

Generally, depending on the intended mode of administration, the pharmaceutically acceptable compositions will contain about 1% to about 99% by weight of a compound(s) of this disclosure, or a pharmaceutically acceptable salt thereof, and 99% to 1% by weight of a suitable pharmaceutical excipient. In one example, the composition will be between about 5% and about 75% by weight of a compound(s) of this disclosure, or a pharmaceutically acceptable salt thereof, with the rest being suitable pharmaceutical excipients.

Actual methods of preparing such dosage forms are known, or will be apparent, to those skilled in this art; for example, see Remington's Pharmaceutical Sciences, 18th Ed., (Mack Publishing Company, Easton, Pa., 1990). The composition to be administered will, in any event, contain a therapeutically effective amount of a compound of this disclosure, or a pharmaceutically acceptable salt thereof, for treatment of a disease-state in accordance with the teachings of this disclosure.

The compounds of this disclosure, or their pharmaceutically acceptable salts, are administered in a therapeutically effective amount which will vary depending upon a variety of factors including the activity of the specific compound employed, the metabolic stability and length of action of the compound, the age, body weight, general health, sex, diet, mode and time of administration, rate of excretion, drug combination, the severity of the particular disease-states, and the host undergoing therapy. The compounds of this disclosure can be administered to a patient at dosage levels in the range of about 0.1 to about 1,000 mg per day. For a normal human adult having a body weight of about 70 kilograms, a dosage in the range of about 0.01 to about 100 mg per kilogram of body weight per day is an example. The specific dosage used, however, can vary. For example, the dosage can depend on a number of factors including the requirements of the patient, the severity of the condition being treated, and the pharmacological activity of the compound being used. The determination of optimum dosages for a particular patient is well known to one of ordinary skill in the art.

The compositions will include a conventional pharmaceutical carrier or excipient and a compound of this disclosure as the/an active agent, and, in addition, can include other medicinal agents and pharmaceutical agents. Compositions of the compounds in this disclosure can be used in combination with anticancer and/or other agents that are generally administered to a patient being treated for cancer, e.g. surgery, radiation and/or chemotherapeutic agent(s). Chemotherapeutic agents that can be useful for administration in combination with compounds of Formula I in treating cancer include alkylating agents, platinum containing agents.

If formulated as a fixed dose, such combination products employ the compounds of this disclosure within the dosage range described above and the other pharmaceutically active agent(s) within its approved dosage range. Compounds of this disclosure can alternatively be used sequentially with known pharmaceutically acceptable agent(s) when a combination formulation is inappropriate.

The compounds described herein, as well as their pharmaceutically acceptable salts or other derivatives thereof, can exist in isotopically-labeled form, in which one or more atoms of the compounds are replaced by an atom having the same atomic number but an atomic mass different from the atomic mass usually found in nature. Examples of isotopes that can be incorporated into compounds of the invention include isotopes of hydrogen, carbon, nitrogen, oxygen, phosphorous, sulfur, fluorine and chloride, such as ²H (deuterium), ³H (tritium), ¹³C, ¹⁴C, ¹⁵N, ¹⁸O, ¹⁷O, ³¹P, ³²P, ³⁵S, ¹⁸F, and ³⁶Cl, respectively. Isotopically labeled compounds of the present invention, as well as pharmaceutically acceptable salts, esters, prodrugs, solvates, hydrates or other derivatives thereof, generally can be prepared by carrying out the procedures disclosed in the Schemes and/or in the Examples and Preparations below, by substituting a readily available isotopically labeled reagent for a non-isotopically labeled reagent.

In the compounds of the invention, unless otherwise stated, any atom not specifically designated as a particular isotope is meant to represent any stable isotope of that atom at its natural abundance. When a position is designated as “H” or “hydrogen”, the position is to be understood to have hydrogen at its natural abundance isotopic composition, with the understanding that some variation of natural isotopic abundance occurs in a synthesized compound depending upon the origin of chemical materials used in the synthesis. When a particular position is designated as “D” or “deuterium”, it is to be understood that the abundance of deuterium at that position is substantially greater than the natural abundance of deuterium, which is 0.015%, and typically has at least 50% deuterium incorporation at that position.

The methods disclosed herein also include methods of treating diseases by administering deuterated compounds of the invention or other isotopically-labeled compounds of the invention alone or as pharmaceutical compositions. In some of these situations, substitution of hydrogen atoms with heavier isotopes such as deuterium can afford certain therapeutic advantages resulting from greater metabolic stability (for example, increased in vivo half-life or reduced dosage requirements).

Moreover, certain isotopically-labeled compounds, for example those into which radioactive isotopes such as ³H and ¹⁴C are incorporated, are useful in drug and/or substrate tissue distribution assays such as positron emission tomagraphy (PET). Tritiated, (³H) and carbon-14 (¹⁴C) isotopes are useful for these embodiments because of their detectability.

### DEFINITIONS

Terms used herein may be preceded and/or followed by a single dash, “—”, or a double dash, “═”, to indicate the bond order of the bond between the named substituent and its parent moiety; a single dash indicates a single bond and a double dash indicates a double bond. In the absence of a single or double dash it is understood that a single bond is formed between the substituent and its parent moiety; further, substituents are intended to be read “left to right” unless a dash indicates otherwise. For example, C₁-C₆alkoxycarbonyloxy and —OC(O)OC₁-C₆alkyl indicate the same functionality. Also, for instance, when variable R⁵ of formula I is defined as —OR⁶, the bond is only to indicate attachment points and the bond is not meant to add additional bonds to the parent structure.

“Administration” and variants thereof (e.g., “administering” a compound) in reference to a compound of the invention means introducing the compound or a prodrug of the compound into the system of the animal in need of treatment. When a compound of the invention or prodrug thereof is provided in combination with one or more other active agents (e.g., surgery, radiation, chemotherapy, and the like), “administration” and its variants are each understood to include concurrent and sequential introduction of the compound or prodrug thereof and other agents.

“Alkoxy” means the group —OR wherein R is alkyl, as defined herein. Representative examples include methoxy, ethoxy, propoxy, butoxy, pentyloxy, hexyloxy, 4-methylhexyloxy, 4-methylheptyloxy, 4,7-dimethyloctyloxy, and the like.

“Alkoxycarbonyl” means an alkoxy group, as defined herein, appended to a parent moiety via a carbonyl group (i.e., a group of the form, —C(O)OR⁰, wherein R⁰ is alkyl, as defined herein). Examples of alkoxycarbonyl groups include, but are not limited to, methoxycarbonyl, ethoxycarbonyl, isopropoxycarbonyl, t-butoxycarbonyl, and n-hexylcarbonyl.

“Alkyl” means a linear or branched hydrocarbon group having from 1 to 10 carbon atoms unless otherwise defined. Representative examples for alkyl groups include methyl, ethyl, propyl, butyl, pentyl, hexyl, 4-methylhexyl, 4-methylheptyl, 4,7-dimethyloctyl, and the like. —(C₁-C₄)alkyl, which means exactly the same as (C₁₋₄)alkyl, includes groups selected from methyl, ethyl, propyl, isopropyl, butyl, sec-butyl,

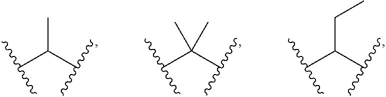

isobutyl, and tert-butyl.

“Alkylamino” means an alkyl group, as defined herein, appended to a parent moiety through an —NH— group (i.e., substituents of the form —N(H)R⁰, where R⁰ is an alkyl group). Examples of alkylamino groups include, but are not limited to, methylamino, ethylamino, isopropylamino, hexylamino, and the like.

“Alkylaminocarbonyl” means an alkylamino group, as defined herein, appended to a parent moiety via a carbonyl group (i.e., a group of the form, —C(O)N(H)R⁰, wherein R⁰ is alkyl, as defined herein). Examples of alkylaminocarbonyl groups include, but are not limited to, methylaminocarbonyl, ethylaminocarbonyl, isopropylaminocarbonyl, t-butylaminocarbonyl, and n-hexylaminocarbonyl.

“Amino” means a —NH₂ group.

“Aryl” means a monovalent, monocyclic, or polycyclic radical having 6 to 14 ring carbon atoms. The monocyclic aryl radical is aromatic and whereas the polycyclic aryl radical may be partially saturated, where at least one of the rings comprising a polycyclic radical is aromatic. The polycyclic aryl radical includes fused, bridged, and spiro ring systems. Unless stated otherwise, the valency may be located on any atom of any ring of the aryl group, valency rules permitting. Representative examples include phenyl, naphthyl, indanyl, and the like.

“Carbonyl” means a —C(O)— group.

“Cycloalkyl” means a monocyclic or polycyclic hydrocarbon radical having 3 to 13 carbon ring atoms. The cycloalkyl radical may be saturated or partially unsaturated, but cannot contain an aromatic ring. The cycloalkyl radical includes fused, bridged and spiro ring systems. Examples of such radicals include cyclopropyl, cyclobutyl, cyclopentyl and cyclohexyl.

“Dialkylamino” means two alkyl groups, each independently as defined herein, appended to a parent moiety through a nitrogen atom (i.e., substituents of the form —N(R⁰)₂, where each R⁰ is an alkyl group). Examples of dialkylamino groups include, but are not limited to N,N-dimethylamino, N,N-diethylamino, N-isopropyl-N-methylamino, N-ethyl-N-hexylamino, and the like.

“Di(C₁-C₄alkyl)aminocarbonyl” means a dialkylamino group, as defined herein, appended to a parent moiety via a carbonyl group (i.e., a group of the form, —C(O)N(R)₂, wherein each R⁰ is alkyl, as defined herein). Examples of dialkylamino groups include, but are not limited to N,N-dimethylaminocarbonyl, N,N-diethylaminocarbonyl, N-isopropyl-N-methylaminocarbonyl, N-ethyl-N-hexylaminocarbonyl, and the like.

“gem-cyclopropyl” means any alkyl group that has a carbon substituted in such a way to form the following structure:

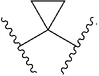 [SMILES: *C1(*)CC1]

“Fused ring system” and “fused ring” refer to a polycyclic ring system that contains bridged or fused rings; that is, where two rings have more than one shared atom in their ring structures. In this application, fused-polycyclics and fused ring systems are not necessarily all aromatic ring systems. Typically, but not necessarily, fused-polycyclics share a vicinal set of atoms, for example naphthalene or 1,2,3,4-tetrahydro-naphthalene. The fused ring structure may contain heteroatoms and may be optionally substituted with one or more groups. It should additionally be noted that saturated carbons of such fused groups (i.e., saturated ring structures) can contain two substitution groups.

“Halo” and “halogen” mean a fluoro, chloro, bromo or iodo group.

“Haloalkyl” means an alkyl radical, as defined herein, substituted with one or more halo atoms. For example, halo-substituted (C₁₋₄)alkyl includes trifluoromethyl, 2,2-dichloroethyl, 2,2,2-trifluoroethyl, perchloroethyl, 2-bromopropyl, and the like.

“Heteroaryl” means a monovalent monocyclic or polycyclic radical having 5 to 14 ring atoms of which one or more of the ring atoms, for example one, two, three, or four ring atoms, are heteroatoms independently selected from —O—, —S(O), (n is 0, 1, or 2), —N—, —N(R^{x})—, and the remaining ring atoms are carbon atoms, where R^{x} is hydrogen, alkyl, hydroxy, alkoxy, —C(O)R⁰ or —S(O)₂R⁰, where R⁰ is alkyl. The monocyclic heteroaryl radical is aromatic and whereas the polycyclic heteroaryl radical may be partially saturated, where at least one of the rings comprising a polycyclic radical is aromatic. The polycyclic heteroaryl radical includes fused, bridged and spiro ring systems. Unless stated otherwise, the valency may be located on any atom of any ring of the heteroaryl group, valency rules permitting. In particular, when the point of valency is located on the nitrogen, then R^{x} is absent. More specifically, the term heteroaryl includes, but is not limited to, 1,2,4-triazolyl, 1,3,5-triazolyl, phthalimidyl, pyridinyl, pyrrolyl, imidazolyl, thienyl, furanyl, indolyl, 2,3-dihydro-1H-indolyl (including, for example, 2,3-dihydro-1H-indol-2-yl, 2,3-dihydro-1H-indol-5-yl, and the like), isoindolyl, indolinyl, isoindolinyl, benzimidazolyl, benzodioxol-4-yl, benzofuranyl, cinnolinyl, indolizinyl, naphthyridin-3-yl, phthalazin-3-yl, phthalazin-4-yl, pteridinyl, purinyl, quinazolinyl, quinoxalinyl, tetrazoyl, pyrazolyl, pyrazinyl, pyrimidinyl, pyridazinyl, oxazolyl, isooxazolyl, oxadiazolyl, benzoxazolyl, quinolinyl, isoquinolinyl, tetrahydroisoquinolinyl (including, for example, tetrahydroisoquinolin-4-yl, tetrahydroisoquinolin-6-yl, and the like), 2,3,3a,7a-tetrahydro-1H-isoindolyl, pyrrolo[3,2-c]pyridinyl (including, for example, pyrrolo[3,2-c]pyridin-2-yl, pyrrolo[3,2-c]pyridin-7-yl, and the like), benzopyranyl, thiazolyl, isothiazolyl, thiadiazolyl, benzothiazolyl, benzothienyl, and the N-oxide derivatives thereof.

“Heterocyclyl” means a monovalent, monocyclic or polycyclic hydrocarbon radical having 3 to 13 ring atoms of which one or more of the ring atoms, for example 1, 2, 3 or 4 ring atoms, are heteroatoms independently selected from —O—, —S(O)ₙ— (n is 0, 1, or 2), —N═ and —N(R^{y})— (where R^{y} is hydrogen, alkyl, hydroxy, alkoxy, —C(O)R⁰ or —S(O)₂R⁰, where R⁰ is alkyl, as defined herein), and the remaining ring atoms are carbon. The heterocycloalkyl radical may be saturated or partially unsaturated, but cannot contain an aromatic ring. The heteocycloalkyl radical includes fused, bridged and spiro ring systems. More specifically the term heterocycloalkyl includes, but is not limited to, azetidinyl, pyrrolidinyl, 2-oxopyrrolidinyl, 2,5-dihydro-1H-pyrrolyl, piperidinyl, 4-piperidonyl, morpholinyl, piperazinyl, 2-oxopiperazinyl, tetrahydropyranyl, 2-oxopiperidinyl, thiomorpholinyl, thiamorpholinyl, perhydroazepinyl, pyrazolidinyl, imidazolinyl, imidazolidinyl, dihydropyridinyl, tetrahydropyridinyl, oxazolinyl, oxazolidinyl, isoxazolidinyl, thiazolinyl, thiazolidinyl, quinuclidinyl, isothiazolidinyl, octahydroindolyl, octahydroisoindolyl, decahydroisoquinolyl, tetrahydrofuryl, 1,4-dioxa-8-azaspiro[4.5]decan-8-yl and tetrahydropyranyl, and the N-oxide derivatives thereof.

“Heterocyclylalkyl” means a heterocyclyl group appended to a parent moiety via an alkyl group, as defined herein. Examples of heterocyclylalkyl groups include, but are not limited to, morpholin-4-ylmethyl, 2-(morpholin-4-yl)ethyl, morpholin-2-ylmethyl, 2-(morpholin-2-yl)ethyl, morpholin-3-ylmethyl, 2-(morpholin-3-yl)ethyl, piperazin-1-ylmethyl, 2-(piperazin-1-yl)ethyl, piperidin-1-ylmethyl, 2-(piperidin-1-yl)ethyl, piperidin-2-ylmethyl, 2-(piperidin-2-yl)ethyl, piperidin-4-ylmethyl, 2-(piperidin-4-yl)ethyl, pyrrolidin-1-ylmethyl, 2-(pyrrolidin-1-yl)ethyl, pyrrolidin-2-ylmethyl, 2-(pyrrolidin-2-yl)ethyl.

“Hydroxyalkyl” means an alkyl group, as defined herein, substituted with at least one, for example one, two, or three, hydroxy group(s), provided that if two hydroxy groups are present they are not both on the same carbon atom. Representative examples include, but are not limited to, hydroxymethyl, 2-hydroxyethyl, 2-hydroxypropyl, 3-hydroxypropyl, 1-(hydroxymethyl)-2-methylbutyl, 2-hydroxybutyl, 3-hydroxybutyl, 4-hydroxybutyl, 2,3-dihydroxypropyl, 1-(hydroxymethyl)-2-hydroxyethyl, 2,3-dihydroxybutyl, 3,4-dihydroxybutyl, 2-(hydroxymethyl)-3-hydroxypropyl, 2-hydroxyethylene, 2,3-dihydroxypropyl, 1-(hydroxymethyl)-2-hydroxyethyl, and the like.

The term “optionally substituted” means the substitution may or may not occur and includes instances where said substitution occurs and instances in which it does not. One of ordinary skill in the art would understand that with respect to any molecule described as containing one or more substituents, only sterically practical and/or synthetically feasible compounds are meant to be included. Unless otherwise specified in this specification, when a variable is said to be optionally substituted or substituted with a substituent(s), this is to be understood that this substitution occurs by replacing a hydrogen that is covalently bound to the variable with one these substituent(s). This meaning shall apply to all variables that are stated to be substituted or optionally substituted in the specification.

Polyethylene glycol (PEG) are polymers of ethylene oxide. Polyethylene glycol refers to the polymer with molecular weight less than 50,000. A polymer is made by joining molecules of ethylene oxide and water together in a repeating pattern. Polyethylene glycol has the following structure: —(CH₂—CH₂—O)n-.

“Saturated bridged ring system” refers to a bicyclic or polycyclic ring system that is not aromatic. Such a system may contain isolated or conjugated unsaturation, but not aromatic or heteroaromatic rings in its core structure (but may have aromatic substitution thereon). For example, hexahydro-furo[3,2-b]furan, 2,3,3a,4,7,7a-hexahydro-1H-indene, 7-aza-bicyclo[2.2.1]heptane and 1,2,3,4,4a,5,8,8a-octahydro-naphthalene are all included in the class “saturated bridged ring system.”

“Spiro ring” refers to a ring originating from a particular annular carbon of another ring. For example, as depicted below:

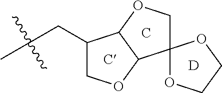

a ring atom of a saturated bridged ring system (rings C and C′), but not a bridgehead atom, can be a shared atom between the saturated bridged ring system and a spiro ring (ring D) attached thereto. A representative example of a spiro ring system is 2,3-dioxa-8-azaspiro[4.5]decan-8-yl.

“Isomers” means compounds having identical molecular formulae but differing in the nature or sequence of bonding of their atoms or in the arrangement of their atoms in space. Isomers that differ in the arrangement of their atoms in space are termed “stereoisomers.” Stereoisomers that are not mirror images of one another are termed “diastereomers” and stereoisomers that are nonsuperimposable mirror images are termed “enantiomers” or sometimes “optical isomers.” A carbon atom bonded to four nonidentical substituents is termed a “chiral center.” A compound with one chiral center has two enantiomeric forms of opposite chirality is termed a “racemic mixture.” A compound that has more than one chiral center has 2ⁿ⁻¹ enantiomeric pairs, where n is the number of chiral centers. Compounds with more than one chiral center may exist as ether an individual diastereomer or as a mixture of diastereomers, termed a “diastereomeric mixture.” When one chiral center is present a stereoisomer may be characterized by the absolute configuration of that chiral center. Absolute configuration refers to the arrangement in space of the substituents attached to the chiral center. Enantiomers are characterized by the absolute configuration of their chiral centers and described by the R- and S-sequencing rules of Cahn, Ingold and Prelog. Conventions for stereochemical nomenclature, methods for the determination of stereochemistry and the separation of stereoisomers are well known in the art (e.g., see “Advanced Organic Chemistry,” 3rd edition, March, Jerry, John Wiley & Sons, New York, 1985). The names and illustration used in this application to describe compounds of the invention, unless indicated otherwise, are meant to be encompassed all possible stereoisomers and any mixture, racemic or otherwise, thereof.

“Metabolite” refers to the break-down or end product of a compound or its salt produced by metabolism or biotransformation in the animal or human body; for example, biotransformation to a more polar molecule such as by oxidation, reduction, or hydrolysis, or to a conjugate (see Goodman and Gilman, “The Pharmacological Basis of Therapeutics” 8.sup.th Ed., Pergamon Press, gilman et al. (eds), 1990 for a discussion of biotransformation). As used herein, the metabolite of a compound of the invention or its salt may be the biologically active form of the compound in the body. In one example, a prodrug may be used such that the biologically active form, a metabolite, is released in vivo. In another example, a biologically active metabolite is discovered serendipitously, that is, no prodrug design per se was undertaken. An assay for activity of a metabolite of a compound of the present invention is known to one of skill in the art in light of the present disclosure.

“Patient” and “subject” for the purposes of the present invention includes humans and other animals, particularly mammals, and other organisms. Thus the methods are applicable to both human therapy and veterinary applications. In another embodiment the patient is a mammal, and in another embodiment the patient is human.

A “pharmaceutically acceptable salt” of a compound means a salt that is pharmaceutically acceptable and that possesses the desired pharmacological activity of the parent compound. It is understood that the pharmaceutically acceptable salts are non-toxic. Additional information on suitable pharmaceutically acceptable salts can be found in Remington's Pharmaceutical Sciences, 17^{th} ed., Mack Publishing Company, Easton, Pa., 1985, or S. M. Berge, et al., “Pharmaceutical Salts,” J. Pharm. Sci., 1977; 66:1-19. It is also understood that the compound can have one or more pharmaceutically acceptable salts associated with it.

Examples of pharmaceutically acceptable acid addition salts include those formed with inorganic acids such as hydrochloric acid, hydrobromic acid, sulfuric acid, nitric acid, phosphoric acid, and the like; as well as organic acids such as acetic acid, trifluoroacetic acid, propionic acid, hexanoic acid, cyclopentanepropionic acid, glycolic acid, pyruvic acid, lactic acid, oxalic acid, maleic acid, malonic acid, succinic acid, fumaric acid, tartaric acid, citric acid, benzoic acid, cinnamic acid, 3-(4-hydroxybenzoyl)benzoic acid, mandelic acid, methanesulfonic acid, ethanesulfonic acid, 1,2-ethanedisulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, p-toluenesulfonic acid, salicylic acid and the like.

Examples of a pharmaceutically acceptable base addition salts include those formed when an acidic proton present in the parent compound is replaced by a metal ion, such as sodium, potassium, lithium, ammonium, calcium, magnesium, iron, zinc, copper, manganese, aluminum salts and the like. Preferable salts are the ammonium, potassium, sodium, calcium and magnesium salts. Salts derived from pharmaceutically acceptable organic non-toxic bases include, but are not limited to, salts of primary, secondary and tertiary amines, substituted amines including naturally occurring substituted amines, cyclic amines and basic ion exchange resins. Examples of organic bases include isopropylamine, trimethylamine, diethylamine, triethylamine, tripropylamine, ethanolamine, 2-dimethylaminoethanol, 2-diethylaminoethanol, dicyclohexylamine, lysine, arginine, histidine, caffeine, procaine, hydrabamine, choline, betaine, ethylenediamine, glucosamine, methylglucamine, theobromine, purines, piperazine, piperidine, N-ethylpiperidine, tromethamine, N-methylglucamine, polyamine resins, and the like. Exemplary organic bases are isopropylamine, diethylamine, ethanolamine, trimethylamine, dicyclohexylamine, choline, and caffeine.

“Prodrug” refers to compounds that are transformed (typically rapidly) in vivo to yield the parent compound of the above formulae, for example, by hydrolysis in blood. Common examples include, but are not limited to, ester and amide forms of a compound having an active form bearing a carboxylic acid moiety. Examples of pharmaceutically acceptable esters of the compounds of this invention include, but are not limited to, alkyl esters (for example with between about one and about six carbons) the alkyl group is a straight or branched chain. Acceptable esters also include cycloalkyl esters and arylalkyl esters such as, but not limited to benzyl. Examples of pharmaceutically acceptable amides of the compounds of this invention include, but are not limited to, primary amides and secondary and tertiary alkyl amides (for example with between about one and about six carbons). Amides and esters of the compounds of the present invention may be prepared according to conventional methods. A thorough discussion of prodrugs is provided in T. Higuchi and V. Stella, “Pro-drugs as Novel Delivery Systems,” Vol 14 of the A.C.S. Symposium Series, and in Bioreversible Carriers in Drug Design, ed. Edward B. Roche, American Pharmaceutical Association and Pergamon Press, 1987, both of which are incorporated herein by reference for all purposes.

“Therapeutically effective amount” is an amount of a compound of the invention, that when administered to a patient, effectively treats the disease. The amount of a compound of the invention which constitutes a “therapeutically effective amount” will vary depending upon a sundry of factors including the activity, metabolic stability, rate of excretion and duration of action of the compound, the age, weight, general health, sex, diet and species of the patient, the mode and time of administration of the compound, the concurrent administration of adjuvants or additional therapies and the severity of the disease for which the therapeutic effect is sought. The therapeutically effective amount for a given circumstance can be determined without undue experimentation.

“Treating” or “treatment” of a disease, disorder, or syndrome, as used herein, includes (i) preventing the disease, disorder, or syndrome from occurring in a human, i.e., causing the clinical symptoms of the disease, disorder, or syndrome not to develop in an animal that may be exposed to or predisposed to the disease, disorder, or syndrome but does not yet experience or display symptoms of the disease, disorder, or syndrome; (ii) inhibiting the disease, disorder, or syndrome, i.e., arresting its development; and (iii) relieving the disease, disorder, or syndrome, i.e., causing regression of the disease, disorder, or syndrome. As is known in the art, adjustments for systemic versus localized delivery, the age, weight, general health, sex, diet and species of the patient, the mode and time of administration of the compound, the concurrent administration of adjuvants or additional therapeutically active ingredients and the severity of the disease for which the therapeutic effect is sought may be necessary, and will be ascertainable with routine experimentation.

The compounds disclosed herein and their pharmaceutically acceptable salts can exist as single stereoisomers, racemates, and as mixtures of enantiomers and diastereomers. The compounds disclosed herein can also exist as geometric isomers. All such single stereoisomers, racemates and mixtures thereof, and geometric isomers are intended to be within the scope of the compounds disclosed herein.

It is assumed that when considering generic descriptions of compounds disclosed herein for the purpose of constructing a compound, such construction results in the creation of a stable structure. That is, one of ordinary skill in the art would recognize that theoretically some constructs which would not normally be considered as stable compounds (that is, sterically practical and/or synthetically feasible, supra).

Methods for the preparation and/or separation and isolation of single stereoisomers from racemic mixtures or non-racemic mixtures of stereoisomers are well known in the art. For example, optically active (R)- and (S)-isomers can be prepared using chiral synthons or chiral reagents, or resolved using conventional techniques. Enantiomers (R- and S-isomers) can be resolved by methods known to one of ordinary skill in the art, for example by: formation of diastereoisomeric salts or complexes which can be separated, for example, by crystallization; via formation of diastereoisomeric derivatives which can be separated, for example, by crystallization, selective reaction of one enantiomer with an enantiomer-specific reagent, for example enzymatic oxidation or reduction, followed by separation of the modified and unmodified enantiomers; or gas-liquid or liquid chromatography in a chiral environment, for example on a chiral support, such as silica with a bound chiral ligand or in the presence of a chiral solvent. It will be appreciated that where a desired enantiomer is converted into another chemical entity by one of the separation procedures described above, a further step can be required to liberate the desired enantiomeric form. Alternatively, specific enantiomer can be synthesized by asymmetric synthesis using optically active reagents, substrates, catalysts or solvents or by converting on enantiomer to the other by asymmetric transformation. For a mixture of enantiomers, enriched in a particular enantiomer, the major component enantiomer can be further enriched (with concomitant loss in yield) by recrystallization.

In addition, the compounds of this disclosure can exist in unsolvated as well as solvated forms with pharmaceutically acceptable solvents such as water, ethanol, and the like. In general, the solvated forms are considered equivalent to the unsolvated forms for the purposes of the compounds of this disclosure.

In addition, it is intended that the present disclosure cover compounds made either using standard organic synthetic techniques, including combinatorial chemistry or by biological methods, such as bacterial digestion, metabolism, enzymatic conversion, and the like.

The examples and scheme below depict the general synthetic procedure for the compounds disclosed herein. Synthesis of the compounds of Formulae I disclosed herein, and embodiments thereof, are not limited by these examples and schemes. One skilled in the art will know that other procedures can be used to synthesize the compounds of Formulae I disclosed herein, and that the procedures described in the examples and schemes is only one such procedure. In the descriptions below, one of ordinary skill in the art would recognize that specific reaction conditions, added reagents, solvents, and reaction temperatures can be modified for the synthesis of specific compounds that fall within the scope of this disclosure. All intermediate compounds described below, for which there is no description of how to synthesize such intermediates within these examples below, are commercially available compounds unless otherwise specified.

### SYNTHETIC EXAMPLES

The following abbreviations and acronyms are used herein and have the indicated meanings throughout:

| | |
|---|---|
| ATP | adenosine triphosphate |
| BOC₂O | di-tert-butyl dicarbonate |
| BOC | t-butyloxycarbonyl |
| br | broad |
| d | doublet |
| dba | dibenzylideneacetone |
| dd | doublet of doublet |
| DIPEA | diisopropylethylamine |
| DMA | N,N-dimethylacetamide |
| DMAP | 4-Dimethylaminopyridine |
| DME | 1,2-dimethoxyethane |
| DMSO | dimethyl sulfoxide |
| dppf | 1,1′-Bis(diphenylphosphino)ferrocene |
| dt | doublet of triplet |
| EDCI | 1-ethyl-3-(3-dimethylaminopropyl)carbodiimide |
| | hydrochloride |
| EI | Electron Impact ionization |
| EtOH | ethanol |
| h or hr | hour(s) |
| HATU | O-(7-azabenzotriazol-1-yl)-n,n,n′,n′-tetramethyluronium |
| | hexafluoro-phosphate |
| HPLC | high pressure liquid chromatography |
| JAK | Janus kinase |
| LDA | lithium diisopropylamide |
| M | molar or molarity |
| m | Multiplet |
| Me | Methyl |
| MeOH | methanol |
| min, Min | minute(s) |
| mmol | millimole(s) |
| MS | mass spectral analysis |
| M/W or MW | microwaves |
| N | normal or normality |
| nBuLi | n-butyl lithium |
| NMP | 1-methyl-2-pyrrolidinone |
| NMR | nuclear magnetic resonance spectroscopy |
| OAc | acetate |
| OiPr | isopropoxide |
| o-tol | 2-methylphenyl |
| Ph | phenyl |
| q | Quartet |
| s | Singlet |
| t or tr | Triplet |
| TEA | triethylamine |
| Tf | 4-trifluoromethylsulfonyl |
| TFA | trifluoroacetic acid |
| THF | tetrahydrofuran |
| Tr | trityl |
| X-phos | 2-Dicyclohexylphosphino-2′,4′,6′-triisopropylbiphenyl |
| Xantphos | 4,5-Bis(diphenylphosphino)-9,9-dimethylxanthene |

### INTERMEDIATES

### Intermediate 1

### N-[3,5-Bis(trifluoromethyl)phenyl]-4-chlorophthalazin-1-amine

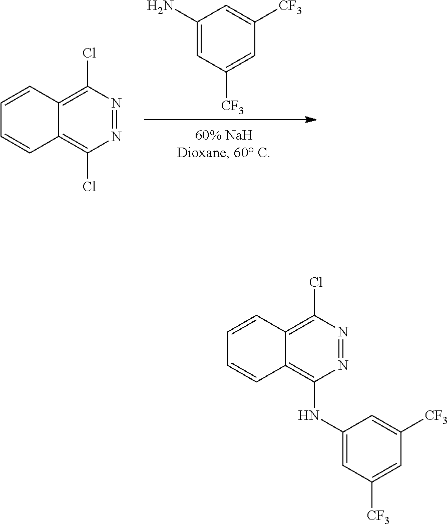 [SMILES: Clc1nnc(Cl)c2ccccc12 ; Nc1cc(C(F)(F)F)cc(C(F)(F)F)c1 ; FC(F)(F)c1cc(Nc2nnc(Cl)c3ccccc23)cc(C(F)(F)F)c1]

1,4-Dichlorophtalazine (500 mg, 2.5 mmol) and 3,5-bis(trifluoromethyl)aniline (592 mg, 2.58 mmol) were suspended in 1,4-dioxane (15 mL) and 60% sodium hydride (in mineral oil) (400 mg, 10 mmol) was carefully added. The mixture was stirred at 60° C. overnight and then was cooled to room temperature. The mixture was carefully quenched with water and then was acidified with 1N aqueous hydrochloric acid (−15 mL) and was extracted with ethyl acetate. The organic portion was washed with brine, was dried over magnesium sulfate, was filtered and was concentrated to afford a yellow solid which was washed with a minimal amount of dichloromethane to afford N-[3,5-bis(trifluoromethyl)phenyl]-4-chlorophthalazin-1-amine as a yellow solid (480 mg, 1.22 mmol, 49% yield). ¹H NMR (400 MHz, CDCl₃): δ 8.31 (m, 3H), 8.16 (m, 1H), 7.91 (m, 2H), 7.45 (s, 1H) MS (EI) for C₁₆H₈ClF₆N₃: 392 (MH⁺), Chlorine isotope pattern.

The following compounds were synthesized in an analogous fashion to the compound described above.

### 4-Chloro-N-[3-methoxy-5-(trifluoromethyl)phenyl]phthalazin-1-amine

MS (EI) for C₁₆H₁₁ClF₃N₃O: 354 (MH⁺), Chlorine isotope pattern.

### Intermediate 2

### Methyl 4-(4-chlorophthalazin-1-yl)benzoate

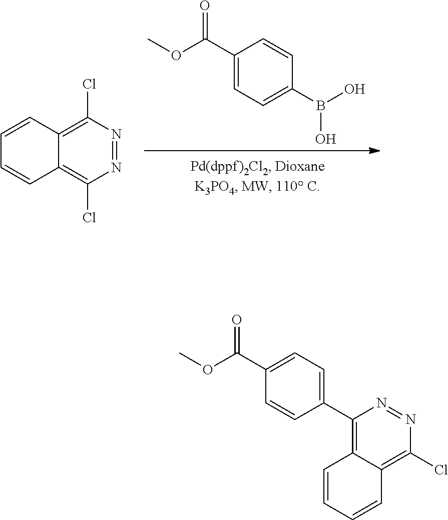 [SMILES: Clc1nnc(Cl)c2ccccc12 ; COC(=O)c1ccc(B(O)O)cc1 ; COC(=O)c1ccc(-c2nnc(Cl)c3ccccc23)cc1]

1,4-Dichlorophtalazine (500 mg, 2.5 mmol), [4-(methoxycarbonyl)phenyl]boronic acid (405 mg, 2.25 mmol), Pd(dppf)₂Cl₂ dichloromethane adduct (100 mg, 0.125 mmol) and potassium phosphate tribasic (1.59 g, 7.5 mmol) were suspended in 1,4-dioxane (4 mL) and water (0.5 mL) and the mixture was irradiated with microwaves at 110° C. for 30 minutes. The mixture was treated with 15 mL of 1N aqueous sodium hydroxide and was extracted with ethyl acetate (2×40 ml). The combined ethyl acetate layer was dried over magnesium sulfate and was concentrated. Purification of the residue on silica gel chromatography (eluted with ethyl acetate:hexanes=1:4 to 1:2) provided methyl 4-(4-chlorophthalazin-1-yl)benzoate as a light brown solid (335 mg, 1.12 mmol, 45% yield). ¹H NMR (400 MHz, CDCl₃): δ 8.43 (d, 1H), 8.25 (d, 1H), 8.04 (m, 2H), 7.94 (m, 1H), 7.81 (d, 2H), 4.02 (s, 3H).

### Intermediate 3

### 1-[4-(4,4,5,5-Tetramethyl-1,3,2-dioxaborolan-2-yl)phenyl]-1,3-dihydro-2H-imidazol-2-one

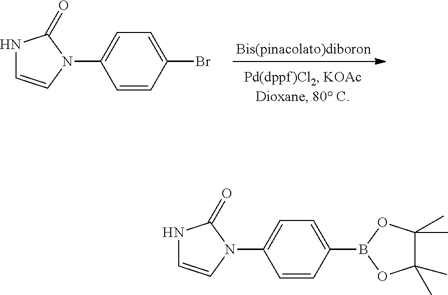 [SMILES: O=c1[nH]ccn1-c1ccc(Br)cc1 ; CC1(C)OB(c2ccc(-n3cc[nH]c3=O)cc2)OC1(C)C]

1-(4-Bromophenyl)-1,3-dihydro-2H-imidazol-2-one (100 mg, 0.418 mmol), bis(pinacolato)diboron (160 mg, 0.627 mmol), Pd(dppf)₂Cl₂ dichloromethane adduct (34 mg, 0.0418 mmol) and potassium acetate (123 mg, 1.25 mmol) were suspended in 1,4-dioxane (10 mL). The mixture was stirred at 80° C. overnight and then was cooled to room temperature. The mixture was diluted with ethyl acetate and was filtered through a celite pad and was concentrated. The residue was purified by column chromatography (eluted with ethyl acetate:dichloromethane=1:1) to give 1-[4-(4,4,5,5-tetramethyl-1,3,2-dioxaborolan-2-yl)phenyl]-1,3-dihydro-2H-imidazol-2-one (71 mg, 60% yield) as an ivory solid. ¹H NMR (400 MHz, CDCl₃): δ 9.95 (br s, 1H), 7.89 (d, 2H), 7.63 (d, 2H), 6.59 (t, 1H), 6.43 (t, 1H), 1.27 (s, 12H).

The following compounds were synthesized in an analogous fashion to the compound described above.

### 1-[4-(4,4,5,5-Tetramethyl-1,3,2-dioxaborolan-2-yl)phenyl]pyrrolidin-2-one

¹H NMR (400 MHz, CDCl₃): δ 7.81 (d, 2H), 7.65 (d, 2H), 3.88 (t, 2H), 2.62 (t, 2H), 2.16 (m, 2H), 1.33 (s, 12H).

### 5-(4,4,5,5-Tetramethyl-1,3,2-dioxaborolan-2-yl)-2-benzofuran-1(3H)-one

¹H NMR (400 MHz, CDCl₃): δ 7.95 (m, 3H), 5.32 (s, 2H), 1.37 (s, 12H).

### 1-[4-(4,4,5,5-Tetramethyl-1,3,2-dioxaborolan-2-yl)phenyl]imidazolidin-2-one

¹H NMR (400 MHz, CDCl₃): δ 7.78 (d, 2H), 7.55 (d, 2H), 5.10 (—NH, br-s, 1H), 3.95 (t, 2H), 3.59 (t, 2H), 1.33 (s, 12H).

### Intermediate 4

### 1-[2-Fluoro-4-(4,4,5,5-tetramethyl-1,3,2-dioxaborolan-2-yl)phenyl]ethanone

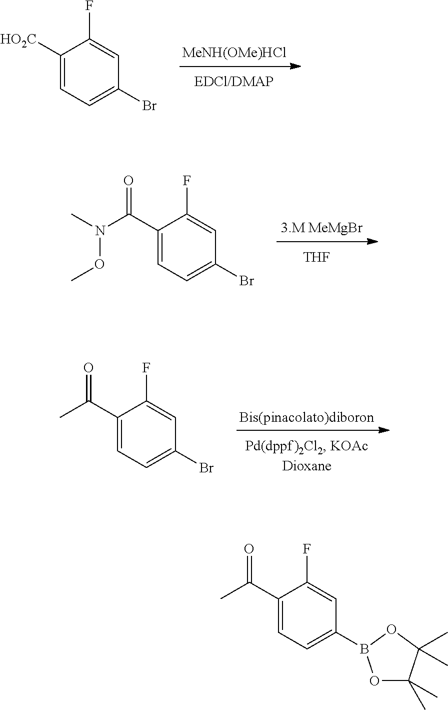 [SMILES: "MeNH(OMe)HCl" ; O=C(O)c1ccc(Br)cc1F ; CON(C)C(=O)c1ccc(Br)cc1F ; CC(=O)c1ccc(Br)cc1F ; "THF" ; CC(=O)c1ccc(B2OC(C)(C)C(C)(C)O2)cc1F]

### 4-Bromo-2-fluoro-N-methoxy-N-methylbenzamide

To a solution of 4-bromo-2-fluorobenzoic acid (2.0 g, 9.13 mmol) in dichloromethane (100 mL) were added EDCI (2.62 g, 13.69 mmol), N,O-dimethylhydroxylamine hydrochloride salt (1.06 g, 10.9 mmol), 4-(dimethylamino)pyridine (1.33 g, 10.9 mmol) and diisopropylethylamine (5 mL). The mixture was stirred at room temperature overnight and then was washed with water, 10% citric acid solution and brine successively. The separated organic layer was dried over magnesium sulfate, was filtered and was concentrated to give 4-bromo-2-fluoro-N-methoxy-N-methylbenzamide (1.97 g, 82% yield) as an oil. ¹H NMR (400 MHz, CDCl₃): δ 7.34 (m, 3H), 3.54 (s, 3H), 3.34 (s, 3H).

### 1-(4-Bromo-2-fluorophenyl)ethanone

To a solution of 4-bromo-2-fluoro-N-methoxy-N-methylbenzamide (1.97 g, 7.5 mmol) in dry tetrahydrofuran (50 mL) was added methyl magnesium bromide (3M in diethyl ether, 5.5 mL) at 0° C. The resulting mixture was allowed to warm to room temperature and then was stirred for 3 h. The reaction mixture was quenched with saturated ammonium chloride, and the organic layer was washed with water and brine, was dried over magnesium sulfate, was filtered and was concentrated to give 1-(4-bromo-2-fluorophenyl)ethanone (1.34 g, 83% yield) as an oil. ¹H NMR (600 MHz, CDCl₃): δ 7.77 (t, 1H), 7.36 (m, 2H), 2.63 (d, 3H).

### 1-[2-Fluoro-4-(4,4,5,5-tetramethyl-1,3,2-dioxaborolan-2-yl)phenyl]ethanone

1-(4-Bromo-2-fluorophenyl)ethanone (500 mg, 2.30 mmol), bis(pinacolato)diboron (877 mg, 3.45 mmol), Pd(dppf)₂Cl₂ dichloromethane adduct (187 mg, 0.230 mmol) and potassium acetate (677 mg, 6.90 mmol) were suspended in 1,4-dioxane (15 mL). The mixture was stirred at 80° C. overnight and then was cooled to ambient temperature. The mixture was diluted with ethyl acetate, was filtered through a celite pad and was concentrated to give 1-[2-fluoro-4-(4,4,5,5-tetramethyl-1,3,2-dioxaborolan-2-yl)phenyl]ethanone (607 mg, 99% yield) as a yellow oil. ¹H NMR (400 MHz, CDCl₃): δ 7.85 (t, 1H), 7.63 (d, 1H), 7.56 (d, 1H), 1.35 (s, 12H).

### Intermediate 5

### 3-[2-(Dimethylamino)ethoxy]-5-(trifluoromethyl)aniline

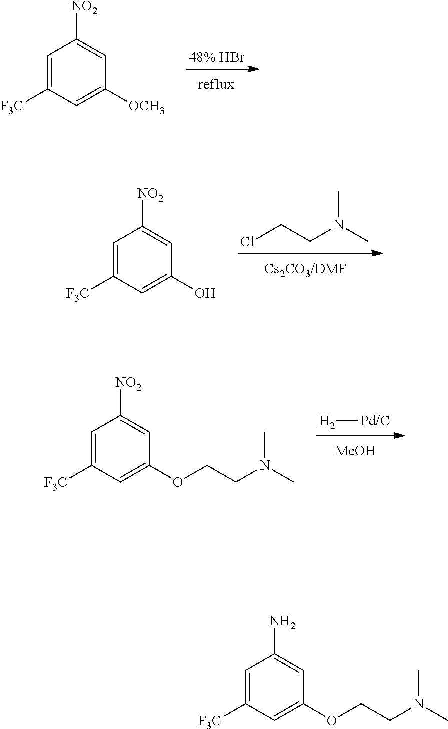

### 3-Nitro-5-(trifluoromethyl)phenol

3-Methoxy-5-nitrobenzotrifluoride (1.0 g, 4.52 mmol) was dissolved in 48% aqueous hydrogen bromide (20 mL) and the solution was heated at reflux overnight. After cooling to room temperature, the residue was dissolved in water and ethyl acetate. The mixture was extracted with ethyl acetate. The organic layer was dried over magnesium sulfate, filtered and was concentrated to give 3-nitro-5-(trifluoromethyl)phenol (950 mg, 93% yield) as a yellow solid. ¹H NMR (400 MHz, CDCl₃): δ 8.07 (s, 1H), 7.88 (t, 1H), 7.43 (s, 1H), 6.22 (br s, 1H).

### N,N-Dimethyl-2-[3-nitro-5-(trifluoromethyl)phenoxy]ethanamine

3-Nitro-5-(trifluoromethyl)phenol (950 mg, 4.58 mmol), 2-(dimethylamino)ethyl chloride hydrochloride (990 mg, 6.88 mmol) and cesium carbonate (4.47 g, 13.7 mmol) were dissolved in N,N-dimethylformamide (15 mL) and the solution was stirred for 4 h at 50° C. After cooling to room temperature, the mixture was dissolved in water and ethyl acetate. The mixture was extracted with ethyl acetate (20 mL×2). The combined organic phase was dried over magnesium sulfate, was filtered and was concentrated. The residue was purified by column chromatography (eluted with dichloromethane:methanol=20:1) to give N,N-dimethyl-2-[3-nitro-5-(trifluoromethyl)phenoxy]ethanamine (700 mg, 55% yield) as a yellow oil. ¹H NMR (400 MHz, CDCl₃): δ 8.08 (s, 1H), 7.93 (t, 1H), 7.49 (s, 1H), 4.19 (t, 2H), 2.82 (t, 2H), 2.42 (s, 6H).

### 3-[2-(Dimethylamino)ethoxy]-5-(trifluoromethyl)aniline

A mixture of N,N-dimethyl-2-[3-nitro-5-(trifluoromethyl)phenoxy]ethanamine (700 mg, 2.5 mmol) and 10% Pd on C (70 mg) in methanol (30 mL) was stirred under hydrogen (45 psi) for 3 h. The reaction mixture was filtered through a celite pad and the filtrate was evaporated to give 3-[2-(dimethylamino)ethoxy]-5-(trifluoromethyl)aniline (520 mg, 84% yield) as a sticky oil. ¹H NMR (400 MHz, CDCl₃): δ 6.54 (s, 1H), 6.50 (s, 1H), 6.34 (t, 1H), 4.06 (t, 2H), 3.82 (br s, 2H), 2.73 (t, 2H), 2.33 (s, 6H).

### Intermediate 6

### 3-[3-(Dimethylamino)propyl]-5-(trifluoromethyl)aniline

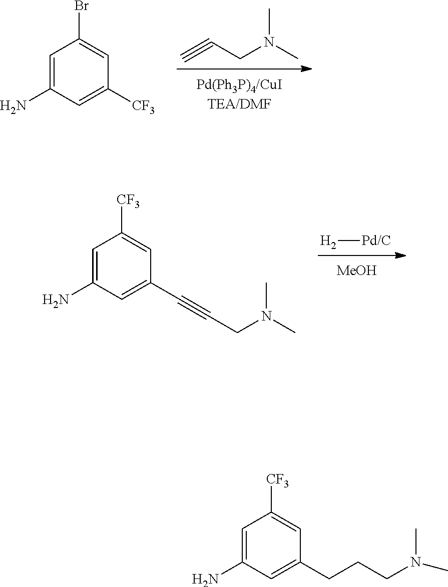

### 3-[3-(Dimethylamino)prop-1-yn-1-yl]-5-(trifluoromethyl)aniline

3-Bromo-5-(trifluoromethyl)aniline (0.5 g, 2.08 mmol) and N,N-dimethylpropargylamine (0.345 g, 4.16 mmol) were dissolved in anhydrous N,N-dimethylformamide and the solution was purged with nitrogen. Triethylamine (0.585 mL, 4.16 mmol), tetrakis(triphenylphosphine)palladium (0) (0.12 g, 0.104 mmol) and copper (I) iodide (0.08 g, 0.416 mmol) were added and the resulting solution was stirred overnight at 60° C. After cooling to room temperature, the residue was dissolved in water and ethyl acetate. The mixture was extracted with ethyl acetate. The combined organic phase was washed with brine, was dried over magnesium sulfate, was filtered and was concentrated. The residue was purified by column chromatography (eluted with dichloromethane:methanol=20:1 to 9:1) to give 3-[3-(dimethylamino)prop-1-yn-1-yl]-5-(trifluoromethyl)aniline (60 mg, 12% yield) as a sticky brown oil. ¹H NMR (400 MHz, CDCl₃): δ 7.08 (s, 1H), 6.89 (s, 1H), 6.83 (s, 1H), 3.87 (br s, 2H), 3.47 (s, 2H), 2.37 (s, 6H).

### 3-[3-(Dimethylamino)propyl]-5-(trifluoromethyl)aniline

A mixture of 3-[3-(dimethylamino)prop-1-yn-1-yl]-5-(trifluoromethyl)aniline (60 mg, 0.247 mmol) and 10% Pd on C (6 mg) in methanol (10 mL) was stirred under hydrogen (45 psi) overnight. The reaction mixture was filtered through a celite pad and the filtrate was evaporated to give 3-[3-(dimethylamino)propyl]-5-(trifluoromethyl)aniline (60 mg, 98% yield) as a yellow sticky oil.

¹H NMR (400 MHz, CDCl₃): δ 6.81 (s, 1H), 6.73 (s, 1H), 6.67 (s, 1H), 3.84 (br s, 2H), 2.62 (t, 2H), 2.55 (t, 2H), 2.40 (s, 6H), 1.87 (m, 2H).

### Intermediate 7

### [4-(5-Methyl-1H-pyrazol-3-yl)phenyl]boronic acid

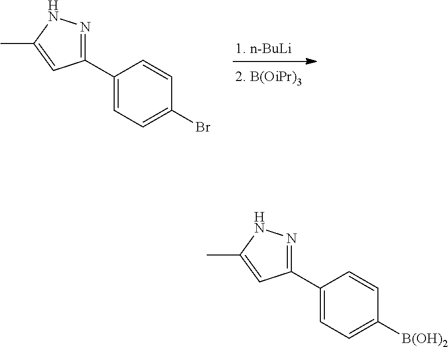

To a 2.5M solution of n-butyllithium (2.02 mL, 5.05 mmol) in dry tetrahydrofuran (10 mL) was added a solution of 3-(4-bromophenyl)-5-methyl-1H-pyrazole (200 mg, 0.842 mmol) in tetrahydrofuran (5 mL) at −78° C. The resulting mixture was stirred at −78° C. for 45 minutes. A solution of triisopropylborate (0.232 mL, 1.01 mmol) was then added and the mixture was stirred at −78° C. for 2 h, and then was allowed to warm to room temperature and was stirred for an additional hour. The mixture was quenched by slow addition of 3% aqueous sodium hydroxide. The resulting aqueous layer was acidified to pH 5-6 by dropwise addition of 3N aqueous hydrochloric acid. The resulting mixture was extracted with ethyl acetate (2×). The combined organic layer was dried over magnesium sulfate, was filtered and was concentrated. The residue was purified by column chromatography (eluted with dichloromethane:methanol=20:1) to give [4-(5-methyl-1H-pyrazol-3-yl)phenyl]boronic acid (98 mg, 57% yield) as a solid. ¹H NMR (400 MHz, CD₃OD): δ 7.52 (d, 2H), 6.80 (d, 2H), 6.16 (s, 1H), 2.25 (s, 3H).

### Intermediate 8

### 3-[(Dimethylamino)methyl]-5-(trifluoromethyl)aniline

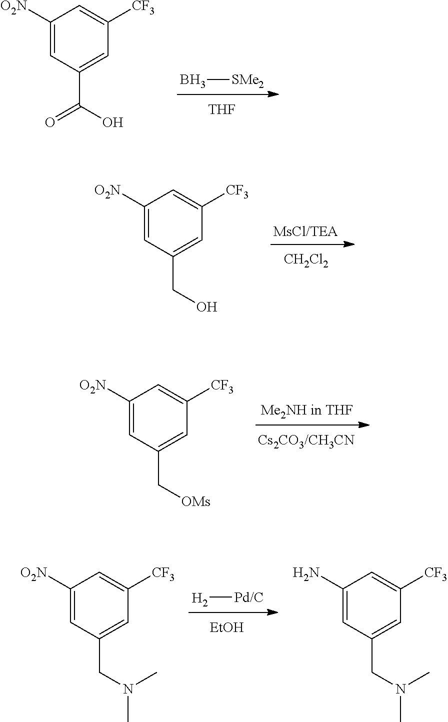

### [3-Nitro-5-(trifluoromethyl)phenyl]methanol

To a solution of 3-nitro-5-(trifluoromethyl)benzoic acid (1.0 g, 4.25 mmol) in distilled tetrahydrofuran (10 mL) was added dropwise borane-methylsulfide (2 mL, 21.3 mmol) at room temperature. The mixture was stirred for 16 h and then was carefully quenched with methanol and was evaporated to give [3-nitro-5-(trifluoromethyl)phenyl]methanol (932 mg, 4.215 mmol, yield=99% yield) as an orange liquid. ¹H NMR (400 MHz, CDCl₃): δ 8.42 (d, 2H), 7.99 (s, 1H), 4.92 (s, 2H).

### 3-Nitro-5-(trifluoromethyl)benzyl methanesulfonate

To a solution of [3-nitro-5-(trifluoromethyl)phenyl]methanol (931 mg, 4.21 mmol) in dichloromethane (10 mL) was added triethylamine (1.8 mL, 12.631 mmol) and methanesulfonyl chloride (0.39 mL, 5.05 mmol) successively at 0° C. The resulting red solution was stirred for an additional 1 h, then was washed with water and brine successively, was dried over magnesium sulfate and was evaporated to give 3-nitro-5-(trifluoromethyl)benzyl methanesulfonate (1.3 g, 4.35 mmol) as a yellow oil. ¹H NMR (400 MHz, CDCl₃): δ 8.50 (d, 2H), 8.01 (s, 1H), 5.38 (s, 2H), 3.14 (s, 3H).

### N,N-Dimethyl-1-[3-nitro-5-(trifluoromethyl)phenyl]methanamine

To a solution of 3-nitro-5-(trifluoromethyl)benzyl methanesulfonate (1.29 g, 4.34 mmol) in acetonitrile (10 mL) was added cesium carbonate (4.24 g, 13.004 mmol) and dimethylamine (2M in tetrahydrofuran, 3.3 ml, 6.502 mmol) at 0° C. The resulting mixture was stirred at room temperature for 2 h and then was filtered through a Celite pad. The filtrate was concentrated and the resulting residue was purified by silica gel column chromatography (dichloromethane:ethyl acetate=10:1 to 1:1, R_{f}=0.3 in hexanes:ethyl acetate=2:1) to afford N,N-dimethyl-1-[3-nitro-5-(trifluoromethyl)phenyl]methanamine (255 mg, 1.03 mmol, 24% yield) as a yellow liquid. ¹H NMR (400 MHz, CDCl₃): δ 8.39 (d, 2H), 7.95 (s, 1H), 3.58 (s, 2H), 2.28 (s, 6H).

### 3-[(Dimethylamino)methyl]-5-(trifluoromethyl)aniline

A mixture of N,N-dimethyl-1-[3-nitro-5-(trifluoromethyl)phenyl]methanamine (251 mg, 1.01 mmol) and 10% Pd on C (25 mg) in ethanol (20 mL) was stirred under hydrogen (45 psi) for 0.5 h. The reaction mixture was filtered through a Celite pad and the filtrate was concentrated to give 3-[(dimethylamino)methyl]-5-(trifluoromethyl)aniline (520 mg, 84% yield) as a yellow oil. ¹H NMR (400 MHz, CDCl₃): δ 6.92 (s, 1H), 6.80 (d, 2H), 3.81 (—NH₂, br-s, 2H), 3.53 (s, 2H).

### Intermediate 9

### Methyl 3-amino-5-(trifluoromethyl)benzoate

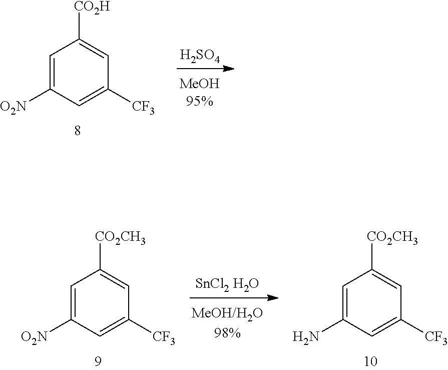

### Methyl 3-nitro-5-(trifluoromethyl)benzoate

3-Nitro-5-(trifluoromethyl)benzoic acid (8) (1.0 g, 4.52 mmol) was dissolved in methanol (30 mL) and a drop of sulfuric acid was added to the solution. The solution was heated at reflux overnight. After cooling to room temperature, the solvent was evaporated and the residue was extracted with ethyl acetate and water. The organic solution was washed with 1N aqueous sodium hydroxide (×2), was dried over magnesium sulfate, was filtered and was concentrated to give methyl 3-nitro-5-(trifluoromethyl)benzoate (1.01 g, 95% yield) as a yellow solid. ¹H NMR (400 MHz, CDCl₃): δ 9.05 (s, 1H), 8.68 (s, 1H), 8.63 (s, 1H), 4.04 (s, 3H).

### Methyl 3-amino-5-(trifluoromethyl)benzoate

To a mixture of methyl 3-nitro-5-(trifluoromethyl)benzoate (350 mg, 1.40 mmol) and tin(II) chloride dihydrate (1.58 g, 7.02 mmol) in methanol (20 mL) was added water (1 mL) and the resulting mixture was stirred at 70° C. for 2 h. After cooling to room temperature, the reaction mixture was concentrated and was quenched by the addition of saturated sodium bicarbonate solution. The mixture was extracted with ethyl acetate (3×). The combined organic portion was dried over magnesium sulfate, was filtered and was concentrated to give methyl 3-amino-5-(trifluoromethyl)benzoate (300 mg, 98% yield) as a colorless solid. ¹H NMR (400 MHz, CDCl₃): δ 7.65 (s, 1H), 7.48 (s, 1H), 7.05 (s, 1H), 3.99 (br s, 2H), 3.91 (s, 3H).

### Intermediate 10

### 3-[2-(Dimethylamino)ethyl]-5-(trifluoromethyl)aniline hydrochloride salt

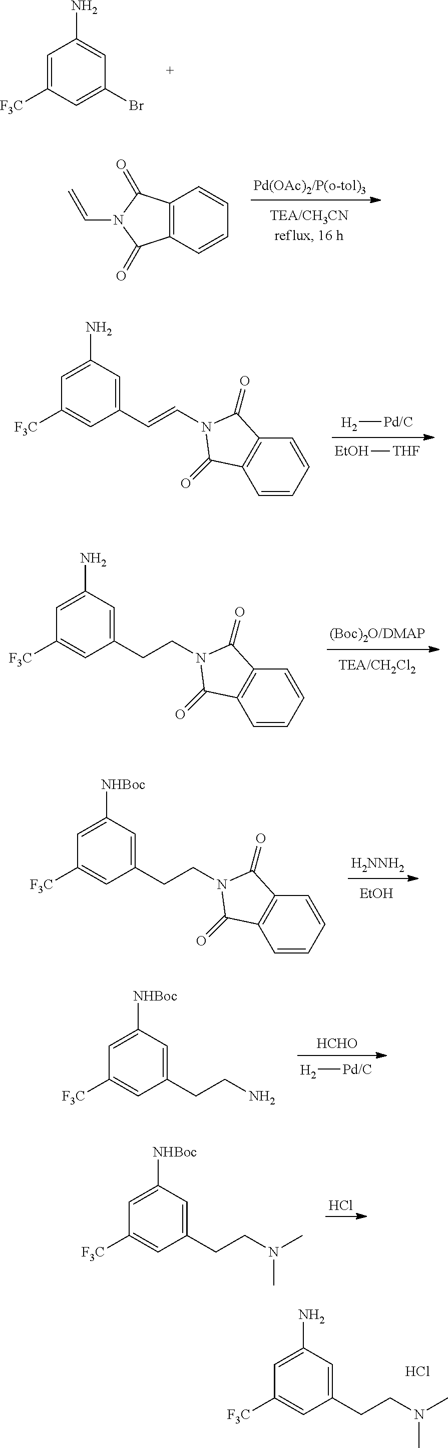

### 2-{(E)-2-[3-Amino-5-(trifluoromethyl)phenyl]vinyl}-1H-isoindole-1,3(2H)-dione

3-Amino-5-bromobenzotrifluoride (2.00 g, 8.332 mmol) was dissolved in acetonitrile (80 mL) and the solution was degassed with nitrogen. Pd(OAc)₂ (94 mg, 0.417 mmol), tris(2-methylphenyl)phosphine (254 mg, 0.833 mmol) and triethylamine (3.5 mL, 25.0 mmol) were added and the bright orange solution was allowed to stir at room temperature for 1 h. To this solution was added N-vinylphthalimide (1.73 g, 10.0 mmol) and the reaction mixture was degassed and was heated at reflux for 16 h. The reaction was filtered through a Celite pad. The filtrate was washed with water and brine, was dried over magnesium sulfate, was filtered and was concentrated. The residue was purified by column chromatography (eluted with hexanes:ethyl acetate=3:1 to 2:1 to 1:1) to afford 2-{(E)-2-[3-amino-5-(trifluoromethyl)phenyl]vinyl}-1H-isoindole-1,3(2H)-dione (2.02 g, 73% yield) as a yellow solid. ¹H NMR (400 MHz, CDCl₃): δ 7.92-7.90 (m, 2H), 7.78-7.76 (m, 2H), 7.59 (d, 1H), 7.35 (d, 1H), 7.09 (s, 1H), 6.91 (s, 1H), 6.79 (s, 1H), 3.88 (—NH₂; br s, 2H).

### 2-{2-[3-Amino-5-(trifluoromethyl)phenyl]ethyl}-1H-isoindole-1,3(2H)-dione

2-{(E)-2-[3-Amino-5-(trifluoromethyl)phenyl]vinyl}-1H-isoindole-1,3(2H)-dione (2.02 g, 6.076 mmol) and 10% Pd on C (0.20 g) in ethanol (125 mL) and tetrahydrofuran (90 mL) was allowed to stir under 50 psi of hydrogen at room temperature for 4 h. The mixture was filtered through a Celite pad and was concentrated. The residue was purified by column chromatography (eluted with hexanes:ethyl acetate=3:1) to give 2-{2-[3-amino-5-(trifluoromethyl)phenyl]ethyl}-1H-isoindole-1,3(2H)-dione (1.35 g, 4.041 mmol) as a colorless solid. ¹H NMR (400 MHz, CDCl₃): δ 7.82-7.78 (m, 2H), 7.70-7.67 (m, 2H), 6.82 (s, 1H), 6.72 (s, 2H), 3.87 (t, 2H), 3.79 (—NH₂; br s, 2H), 2.90 (t, 2H).

### tert-Butyl {3-[2-(1,3-dioxo-1,3-dihydro-2H-isoindol-2-yl)ethyl]-5-(trifluoromethyl)phenyl}carbamate

To a solution of 2-{2-[3-amino-5-(trifluoromethyl)phenyl]ethyl}-1H-isoindole-1,3(2H)-dione (300 mg, 0.897 mmol) in dichloromethane (10 mL) was added di-tert-butyl dicarbonate (294 mg, 1.346 mmol) and 4-(dimethylamino)pyridine (110 mg, 0.897 mmol) at 0° C. The resulting solution was stirred for 15 h and was concentrated to give a residue which was purified by silica gel column chromatography (eluted with hexanes:ethyl acetate=3:1, R_{f}=0.4 in hexanes:ethyl acetate=2:1) to afford tert-butyl {3-[2-(1,3-dioxo-1,3-dihydro-2H-isoindol-2-yl)ethyl]-5-(trifluoromethyl)phenyl}carbamate (156 mg, 0.359 mmol, 40% yield) as a viscous liquid. ¹H NMR (400 MHz, CDCl₃): δ 7.83-7.81 (m, 2H), 7.70-7.68 (m, 2H), 7.48 (s, 1H), 7.43 (s, 1H), 7.40 (s, 1H), 3.93 (t, 2H), 3.06 (t, 2H), 1.43 (s, 9H).

### tert-Butyl [3-(2-aminoethyl)-5-(trifluoromethyl)phenyl]carbamate

To a solution of tert-butyl {3-[2-(1,3-dioxo-1,3-dihydro-2H-isoindol-2-yl)ethyl]-5-(trifluoromethyl)phenyl}carbamate (151 mg, 0.348 mmol) in ethanol (3 mL) was added hydrazine monohydrate (0.034 mL, 0.695 mmol). The resulting solution was stirred at 100° C. for 1 h and then was concentrated to give a residue. Silica gel column chromatography (eluted with methanol:dichloromethane=1:10 to 1:5) gave tert-butyl [3-(2-aminoethyl)-5-(trifluoromethyl)phenyl]carbamate (67 mg, 0.220 mmol, 63% yield) as a solid. ¹H NMR (400 MHz, CD₃OD): δ 7.65 (s, 1H), 7.53 (s, 1H), 7.15 (s, 1H), 2.98 (t, 2H), 2.85 (t, 2H), 1.51 (s, 9H).

### tert-Butyl {3-[2-(dimethylamino)ethyl]-5-(trifluoromethyl)phenyl}carbamate

To a mixture of tert-butyl [3-(2-aminoethyl)-5-(trifluoromethyl)phenyl]carbamate (67 mg, 0.220 mmol) and 10% Pd on C (7 mg) in methanol (5 mL) was added 37% formaldehyde (0.1 mL, 1.32 mmol) and the resulting mixture was stirred under hydrogen (45 psi) for 16 h. The reaction mixture was filtered through a Celite pad and the filtrate was concentrated to give a residue. Silica gel column chromatography (eluted with methanol:dichloromethane=1:10) afforded tert-butyl {3-[2-(dimethylamino)ethyl]-5-(trifluoromethyl)phenyl}carbamate (18 mg, 0.054 mmol, 25% yield) as a solid. ¹H NMR (400 MHz, CDCl₃): δ 7.48 (s, 1H), 7.38 (s, 1H), 7.10 (s, 1H), 6.6 (—NH, s, 1H), 2.80 (t, 2H), 2.55 (t, 2H), 2.30 (s, 6H), 1.50 (s, 9H).

### 3-[2-(Dimethylamino)ethyl]-5-(trifluoromethyl)aniline hydrochloride salt

To a solution of tert-butyl {3-[2-(dimethylamino)ethyl]-5-(trifluoromethyl)phenyl}carbamate (31 mg, 0.093 mmol) in methanol was added 2M hydrochloric acid in diethyl ether (1 mL, 1.87 mmol) dropwise. The mixture was stirred at room temperature for 16 h. And then it was concentrated to give 3-[2-(dimethylamino)ethyl]-5-(trifluoromethyl)aniline hydrochloride salt (25 mg, quantitative yield) as a pale yellow solid. ¹H NMR (400 MHz, CD₃OD): δ 7.78 (s, 1H), 7.67 (s, 1H), 7.58 (s, 1H), 3.40 (m, 2H), 3.23 (m, 2H), 2.92 (s, 6H).

### Intermediate 11

### 3-(1-Methylpiperidin-4-yl)-5-(trifluoromethyl)aniline

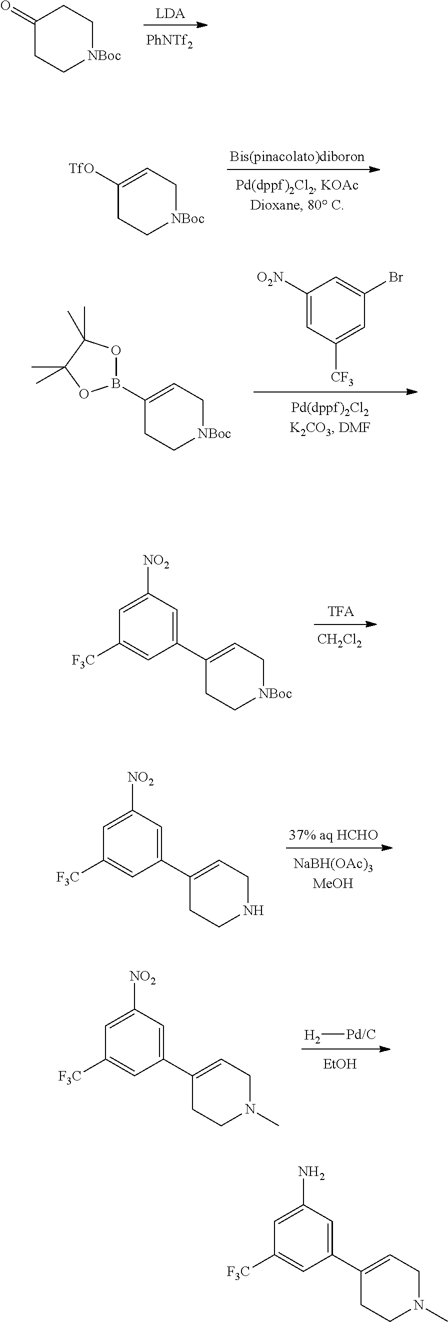

### tert-Butyl 4-{[(trifluoromethyl)sulfonyl]oxy}-3,6-dihydropyridine-1(2H)-carboxylate

To a solution of diisopropylamine (0.847 mL, 6.00 mmol) in dry tetrahydrofuran (15 mL) at −60° C. was added n-butyllithium (1.6M in hexane; 3.75 mL, 6.00 mmol) under nitrogen atmosphere. The reaction mixture was stirred for 5 min at −60° C. A solution of tert-butyl 4-oxopiperidine-1-carboxylate (1.0 g, 5.00 mmol) in dry tetrahydrofuran (20 mL) was added, and the reaction mixture was stirred for 10 min. Then a solution of N-phenyltrifluoromethanesulfonamide (1.96 g, 5.50 mmol) was added. The reaction mixture was stirred at −60° C. for 30 min and the mixture was allowed to warm to room temperature and was stirred for 2 h. The reaction was quenched with saturated sodium bicarbonate, followed by extraction with ethyl acetate. The organic layer was washed sequentially with 5% citric acid solution, 1N aqueous sodium hydroxide, water and brine, was dried over magnesium sulfate, was filtered and was concentrated. The residue was purified by column chromatography (eluted with ethyl acetate:hexanes=1:10) to give tert-butyl 4-{[(trifluoromethyl)sulfonyl]oxy}-3,6-dihydropyridine-1(2H)-carboxylate (1.2 g, 72% yield) as a colorless oil. ¹H NMR (400 MHz, CDCl₃): δ 5.73 (s, 1H), 4.01 (s, 2H), 3.59 (t, 2H), 2.40 (s, 2H), 1.43 (s, 9H).

### tert-Butyl 4-(4,4,5,5-tetramethyl-1,3,2-dioxaborolan-2-yl)-3,6-dihydropyridine-1(2H)-carboxylate

tert-Butyl 4-{[(trifluoromethyl)sulfonyl]oxy}-3,6-dihydropyridine-1(2H)-carboxylate (460 mg, 1.38 mmol), bis(pinacolato)diboron (458 mg, 1.80 mmol), Pd(dppf)₂Cl₂ dichloromethane adduct (56 mg, 0.069 mmol) and potassium acetate (408 mg, 1.46 mmol) were suspended in 1,4-dioxane (20 mL). The mixture was stirred at 80° C. overnight and then was cooled to room temperature. The mixture was diluted with ethyl acetate, was filtered through a Celite pad and was concentrated. The residue was purified by column chromatography (eluted with ethyl acetate:hexanes=1:10) to give tert-butyl 4-(4,4,5,5-tetramethyl-1,3,2-dioxaborolan-2-yl)-3,6-dihydropyridine-1(2H)-carboxylate (335 mg, 78% yield) as a colorless solid. ¹H NMR (400 MHz, CDCl₃): δ 5.50 (s, 1H), 3.94 (s, 2H), 3.45 (t, 2H), 2.21 (s, 2H), 1.45 (s, 9H), 1.25 (s, 12H).

### tert-Butyl 4-[3-nitro-5-(trifluoromethyl)phenyl]-3,6-dihydropyridine-1(2H)-carboxylate

tert-Butyl 4-(4,4,5,5-tetramethyl-1,3,2-dioxaborolan-2-yl)-3,6-dihydropyridine-1(2H)-carboxylate (200 mg, 0.646 mmol), 1-bromo-3-nitro-5-(trifluoromethyl)benzene (261 mg, 0.969 mmol), Pd(dppf)₂Cl₂ dichloromethane adduct (52 mg, 0.064 mmol) and potassium carbonate (268 mg, 1.94 mmol) were suspended in anhydrous N,N-dimethylformamide (5 mL). The mixture was stirred at 80° C. overnight and then was cooled to room temperature. The mixture was partitioned between ethyl acetate and water. The organic layer was dried over magnesium sulfate, was filtered and was concentrated. The residue was purified by column chromatography (eluted with ethyl acetate:Hexanes=1:10) to give tert-butyl 4-[3-nitro-5-(trifluoromethyl)phenyl]-3,6-dihydropyridine-1(2H)-carboxylate (114 mg, 47% yield) as a yellow sticky oil. ¹H NMR (400 MHz, CDCl₃): δ 8.37 (s, 1H), 8.33 (s, 1H), 7.89 (s, 1H), 6.27 (s, 1H), 4.09 (s, 2H), 3.65 (t, 2H), 2.55 (s, 2H), 1.46 (s, 9H).

### 4-[3-Nitro-5-(trifluoromethyl)phenyl]-1,2,3,6-tetrahydropyridine

To a solution of tert-butyl 4-[3-nitro-5-(trifluoromethyl)phenyl]-3,6-dihydropyridine-1(2H)-carboxylate (205 mg, 0.550 mmol) in dichloromethane (10 mL) was added trifluoroacetic acid (0.42 mL, 5.505 mmol). The resulting solution was stirred at room temperature overnight and was evaporated to give a residue. Silica gel column chromatography (eluted with methanol:dichloromethane=1:20 to 1:10) gave 4-[3-nitro-5-(trifluoromethyl)phenyl]-1,2,3,6-tetrahydropyridine (212 mg, quantitative yield) as a light yellow foam. ¹H NMR (400 MHz, CD₃OD): δ 9.78 (s, 1H), 8.45 (s, 1H), 8.41 (s, 1H), 7.92 (s, 1H), 6.27 (s, 1H), 3.92 (s, 2H), 3.50 (t, 2H), 2.88 (s, 2H).

### 1-Methyl-4-[3-nitro-5-(trifluoromethyl)phenyl]-1,2,3,6-tetrahydropyridine

To a solution of 4-[3-nitro-5-(trifluoromethyl)phenyl]-1,2,3,6-tetrahydropyridine (200 mg, 0.541 mmol) dissolved in methanol (10 mL) was added 37% aqueous formaldehyde (0.08 mL, 1.08 mmol,) and sodium triacetoxyborohydride (228 mg, 1.08 mmol). The resulting solution was stirred at room temperature for 1 h and was concentrated to give a residue. Silica gel column chromatography (eluted with methanol:dichloromethane=1:10) gave 1-methyl-4-[3-nitro-5-(trifluoromethyl)phenyl]-1,2,3,6-tetrahydropyridine (95 mg, 61% yield) as a light yellow solid. ¹H NMR (400 MHz, CD₃OD): δ 8.41 (s, 1H), 8.35 (s, 1H), 7.93 (s, 1H), 6.32 (s, 1H), 3.26 (s, 2H), 2.80 (t, 2H), 2.67 (s, 2H), 2.50 (s, 3H).

### 3-(1-Methylpiperidin-4-yl)-5-(trifluoromethyl)aniline

A mixture of 1-methyl-4-[3-nitro-5-(trifluoromethyl)phenyl]-1,2,3,6-tetrahydropyridine (95 mg, 0.330 mmol) and 10% Pd on C (30 mg) in ethanol (10 mL) was stirred under hydrogen (45 psi) for 2 h. The reaction mixture was filtered through a Celite pad and the filtrate was concentrated to give 3-(1-methylpiperidin-4-yl)-5-(trifluoromethyl)aniline (80 mg, 93% yield) as a sticky oil. ¹H NMR (400 MHz, CDCl₃): δ 6.78 (s, 1H), 6.71 (s, 1H), 6.67 (s, 1H), 3.28 (d, 2H), 2.52 (m, 4H), 2.31 (t, 2H), 1.94 (m, 2H), 1.85 (d, 2H).

### Intermediate 12

### 1-tert-Butyl 3-ethyl 6-(4,4,5,5-tetramethyl-1,3,2-dioxaborolan-2-yl)-1H-indazole-1,3-dicarboxylate

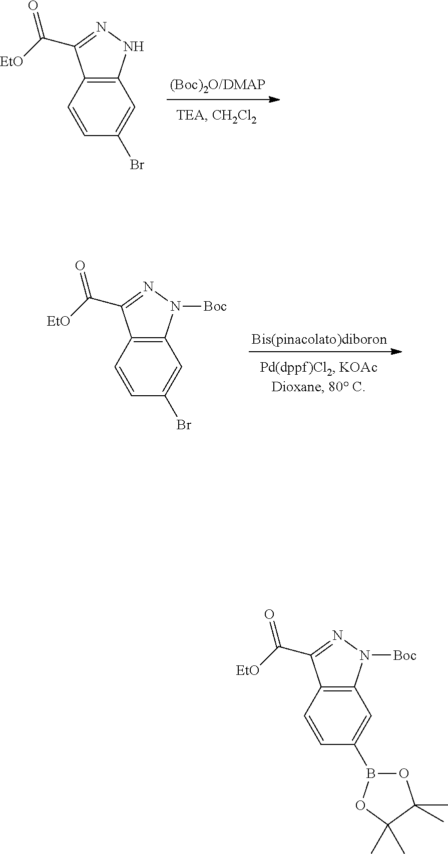 [SMILES: CCOC(=O)c1n[nH]c2cc(Br)ccc12 ; "TEA, CH2Cl2" ; CCOC(=O)c1nn(C(=O)OC(C)(C)C)c2cc(Br)ccc12 ; CCOC(=O)c1nn(C(=O)OC(C)(C)C)c2cc(B3OC(C)(C)C(C)(C)O3)ccc12]

### 1-tert-Butyl 3-ethyl 6-bromo-1H-indazole-1,3-dicarboxylate

To a solution of ethyl 6-bromo-1H-indazole-3-carboxylate (200 mg, 0.743 mmol) dissolved in dichloromethane (50 mL) was added triethylamine (114 mL, 0.818 mmol), di-tert-butyl dicarbonate (324 mg, 1.486 mmol) and 4-(dimethylamino)pyridine (9 mg, 0.074 mmol) successively at 0° C. The mixture was stirred at 0° C. for 1 h and then for 2 h at room temperature. The organic layer was washed successively with 0.5N aqueous hydrochloric acid, water and brine, was dried over magnesium sulfate, was filtered and was concentrated. The crude material was purified by silica gel column chromatography (eluted with hexanes:ethyl acetate=3:1, R_{f}=0.6 in hexanes:ethyl acetate=2:1) to give 1-tert-butyl 3-ethyl 6-bromo-1H-indazole-1,3-dicarboxylate (274 mg, 0.742 mmol, 99% yield) as a pale yellow solid. ¹H NMR (400 MHz, CDCl₃): δ 8.44 (s, 1H), 8.10 (d, 1H), 7.54 (d, 1H), 4.51 (q, 2H), 1.73 (s, 9H), 1.46 (t, 3H).

### 1-tert-Butyl 3-ethyl 6-(4,4,5,5-tetramethyl-1,3,2-dioxaborolan-2-yl)-1H-indazole-1,3-dicarboxylate

1-tert-Butyl 3-ethyl 6-bromo-1H-indazole-1,3-dicarboxylate (274 mg, 0.743 mmol), bis(pinacolato)diboron (335 mg, 1.320 mmol), Pd(dppf)₂Cl₂ dichloromethane adduct (72 mg, 0.088 mmol) and potassium acetate (259 mg, 2.640 mmol) were suspended in 1,4-dioxane (15 mL). The mixture was stirred at 80° C. overnight and then was cooled to room temperature. The mixture was diluted with ethyl acetate, was filtered through a Celite pad and was concentrated. The residue was purified by column chromatography (eluted with hexanes:ethyl acetate=3:1, R_{f}=0.4 in hexanes:ethyl acetate=3:1) to give 1-tert-butyl 3-ethyl 6-(4,4,5,5-tetramethyl-1,3,2-dioxaborolan-2-yl)-1H-indazole-1,3-dicarboxylate (232 mg, 0.557 mmol, 75% yield) as a colorless solid. ¹H NMR (400 MHz, CDCl₃): δ 8.72 (s, 1H), 8.24 (d, 1H), 7.83 (d, 1H), 4.54 (q, 2H), 1.76 (s, 9H), 1.50 (t, 3H), 1.38 (s, 12H).

### Intermediate 13

### 1-[2-Fluoro-4-(4,4,5,5-tetramethyl-1,3,2-dioxaborolan-2-yl)phenyl]propan-1-one

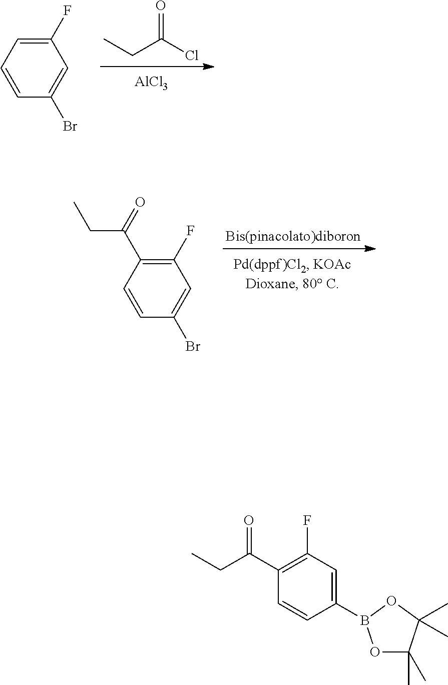

### 1-(4-Bromo-2-fluorophenyl)propan-1-one

A mixture of 3-bromofluorobenzene (5.0 g, 28.57 mmol) and aluminum (III) chloride (11.6 g, 86.99 mmol) was heated under nitrogen until a slurry formed. Propionyl chloride (3.2 g, 34.59 mmol) was added over 15 min and the mixture was heated at 90° C. for 1 h. The reaction was poured onto ice-water (100 mL) and the resulting mixture was extracted with dichloromethane (3×50 mL). The combined organic extract was dried over magnesium sulfate, was filtered, was concentrated and was purified by column chromatography (eluted with hexanes:ethyl acetate=10:1) to give 1-(4-bromo-2-fluorophenyl)propan-1-one as a colorless solid (854 mg, 3.696 mmol, 13% yield). ¹H NMR (400 MHz, CDCl₃): δ 7.75 (t, 1H), 7.38-7.29 (m, 2H), 3.00-2.94 (m, 2H), 1.18 (t, 3H).

### 1-[2-Fluoro-4-(4,4,5,5-tetramethyl-1,3,2-dioxoborolan-2-yl)phenyl]propan-1-one

1-(4-Bromo-2-fluorophenyl)propan-1-one (433 mg, 1.874 mmol), bis(pinacolato)diboron (714 mg, 2.811 mmol), Pd(dppf)₂Cl₂ dichloromethane adduct (153 mg, 0.817 mmol) and potassium acetate (552 mg, 5.622 mmol) were suspended in 1,4-dioxane (20 mL). The mixture was stirred at 80° C. overnight and then was cooled to room temperature. The mixture was diluted with ethyl acetate, was filtered through a Celite pad and was concentrated. The residue was purified by column chromatography (eluted with hexanes:ethyl acetate=10:1) to give 1-[2-fluoro-4-(4,4,5,5-tetramethyl-1,3,2-dioxaborolan-2-yl)phenyl]propan-1-one (103 mg, 0.467 mmol, 25% yield) as a yellow oil. ¹H NMR (400 MHz, CDCl₃): δ 7.82 (t, 1H), 7.61 (d, 1H), 7.53 (d, 1H), 3.00 (q, 2H), 1.35 (s, 12H), 1.20 (t, 3H).

### Example 1

### Methyl 4-(4-{[3,5-bis(trifluoromethyl)phenyl]amino}phthalazin-1-yl)benzoate

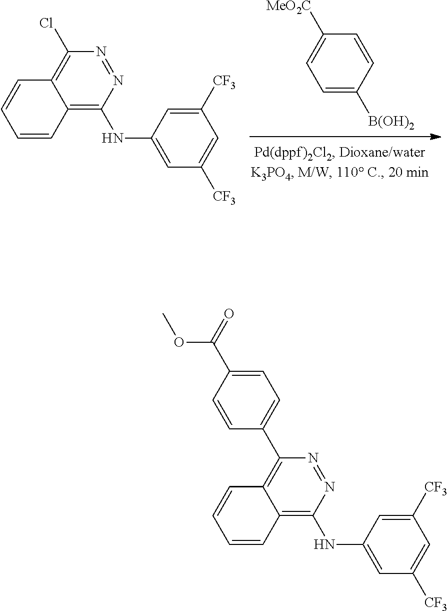 [SMILES: FC(F)(F)c1cc(Nc2nnc(Cl)c3ccccc23)cc(C(F)(F)F)c1 ; COC(=O)c1ccc(B(O)O)cc1 ; COC(=O)c1ccc(-c2nnc(Nc3cc(C(F)(F)F)cc(C(F)(F)F)c3)c3ccccc23)cc1]

N-[3,5-Bis(trifluoromethyl)phenyl]-4-chlorophthalazin-1-amine (prepared according to the procedure for Intermediate 1) (1.27 g, 3.245 mmol), [4-(methoxycarbonyl)phenyl]boronic acid (876 mg, 4.867 mmol), Pd(dppf)₂Cl₂ dichloromethane adduct (265 mg, 0.325 mmol) and potassium phosphate tribasic (1.38 g, 6.493 mmol) were suspended in 1,4-dioxane (15 mL) and water (1.5 mL) and the mixture was irradiated with microwaves at 110° C. for 20 minutes. The mixture was treated with 1N aqueous sodium hydroxide (10 mL) and was extracted with ethyl acetate. The ethyl acetate layer was washed with 1N aqueous sodium hydroxide (2×10 mL), was dried over magnesium sulfate, was filtered and was concentrated. The residue was washed with a minimal amount of dichloromethane and diethyl ether to afford methyl 4-(4-{[3,5-bis(trifluoromethyl)phenyl]amino}phthalazin-1-yl)benzoate as a pale brown solid (1.6 g, 0.324 mmol, 99% yield). ¹H NMR (400 MHz, d₆-DMSO): δ 9.97 (s, 1H), 8.84 (s, 2H), 8.72 (d, 1H), 8.19-8.14 (m, 3H), 8.04 (t, 1H), 7.95 (d, 1H), 7.88 (d, 2H), 7.71 (s, 1H), 3.93 (s, 3H). MS (EI) for C₂₄H₁₅F₆N₃O₂: 492 (MH⁺).

The following compounds were synthesized in an analogous fashion to the compound described above.

### 1-[4-(4-{[3,5-Bis(trifluoromethyl)phenyl]amino}phthalazin-1-yl)phenyl]-1,3-dihydro-2H-imidazol-2-one

Using 1-[4-(4,4,5,5-tetramethyl-1,3,2-dioxaborolan-2-yl)phenyl]-1,3-dihydro-2H-imidazol-2-one (prepared according to the procedure described for Intermediate 3). ¹H NMR (400 MHz, d₆-DMSO): δ 10.38 (s, 1H), 9.88 (s, 1H), 8.80 (s, 2H), 8.67 (d, 1H), 8.09 (t, 1H), 7.96 (m, 4H), 7.73 (d, 2H), 7.66 (s, 1H), 7.09 (t, 1H), 6.64 (t, 1H). MS (EI) for C₂₅H₁₅F₆N₅O: 516 (MH⁺).

### N-[3,5-Bis(trifluoromethyl)phenyl]-4-(1H-indazol-6-yl)phthalazin-1-amine

MS (EI) for C₂₃H₁₃F₆N₅: 474 (MH⁺).

### N-[3,5-Bis(trifluoromethyl)phenyl]-4-[4-(1H-pyrazol-3-yl)phenyl]phthalazin-1-amine

MS (EI) for C₂₅H₁₅F₆N₅: 500 (MH⁺).

### N-[3,5-Bis(trifluoromethyl)phenyl]-4-[4-(5-methyl-1H-pyrazol-3-yl)phenyl]phthalazin-1-amine

Using [4-(5-methyl-1H-pyrazol-3-yl)phenyl]boronic acid (prepared according to the procedure described for Intermediate 7). ¹H NMR (600 MHz, d₆-DMSO): δ 12.63 (s, 1H), 9.88 (s, 1H), 8.80 (s, 2H), 8.67 (d, 1H), 8.10 (m, 1H), 7.98 (m, 4H), 7.68 (m, 3H), 6.52 (s, 1H), 2.26 (s, 3H). MS (EI) for C₂₆H₁₇F₆N₅: 514 (MH⁺).

### 1-[4-(4-{[3,5-Bis(trifluoromethyl)phenyl]amino}phthalazin-1-yl)phenyl]pyrrolidin-2-one

Using 1-[4-(4,4,5,5-tetramethyl-1,3,2-dioxaborolan-2-yl)phenyl]pyrrolidin-2-one (prepared according to the procedure described for Intermediate 3). ¹H NMR (400 MHz, d₆-DMSO): δ 9.91 (s, 1H), 8.84 (s, 2H), 8.71 (d, 1H), 8.14 (t, 1H), 8.01 (m, 2H), 7.90 (d, 2H), 7.71 (m, 3H), 3.96 (t, 2H), 2.56 (t, 2H), 2.12 (m, 2H). MS (EI) for C₂₆H₁₈F₆N₄O: 517 (MH⁺).

### Methyl 4-(4-{[3-(methyloxy)-5-(trifluoromethyl)phenyl]amino}phthalazin-1-yl)benzoate

Using 4-chloro-N-[3-methoxy-5-(trifluoromethyl)phenyl]phthalazin-1-amine (prepared according to the procedure described for Intermediate 1). MS (EI) for C₂₄H₁₈F₃N₃O₃: 454 (MH⁺).

### 4-(4-{[3,5-Bis(trifluoromethyl)phenyl]amino}phthalazin-1-yl)benzamide

MS (EI) for C₂₃H₁₄F₆N₄O: 477 (MH⁺).

### Methyl 4-(4-{[3,5-bis(trifluoromethyl)phenyl]amino}phthalazin-1-yl)-3-methylbenzoate

¹H NMR (600 MHz, d₆-DMSO): δ 9.93 (s, 1H), 8.83 (s, 2H), 8.70 (d, 1H), 8.08 (t, 1H), 8.00 (s, 1H), 7.93 (m, 2H), 7.68 (s, 1H), 7.47 (d, 1H), 7.41 (d, 1H) 3.88 (s, 3H), 2.06 (s, 3H). MS (EI) for C₂₅H₁₇F₆N₃O₂: 506 (MH⁺).

### 4-(4-{[3,5-Bis(trifluoromethyl)phenyl]amino}phthalazin-1-yl)benzaldehyde

¹H NMR (600 MHz, d₆-DMSO): δ 10.17 (s, 1H), 9.99 (s, 1H), 8.85 (s, 2H), 8.74 (d, 1H), 8.20-8.11 (m, 3H), 8.03 (t, 1H), 7.95 (m, 3H), 7.72 (s, 1H). MS (EI) for C₂₃H₁₃F₆N₃O: 462 (MH⁺).

### 5-(4-{[3,5-Bis(trifluoromethyl)phenyl]amino}phthalazin-1-yl)-2-benzofuran-1(3H)-one

Using 5-(4,4,5,5-tetramethyl-1,3,2-dioxaborolan-2-yl)-2-benzofuran-1(3H)-one (prepared according to the procedure described for Intermediate 3). ¹H NMR (600 MHz, d₆-DMSO): δ 10.00 (s, 1H), 8.85 (s, 2H), 8.74 (d, 1H), 8.15 (t, 1H), 8.05 (m, 3H), 7.95 (m, 2H), 7.72 (s, 1H), 5.55 (s, 2H). MS (EI) for C₂₄H₁₃F₆N₃O₂: 490 (MH⁺).

### N-[3,5-Bis(trifluoromethyl)phenyl]-4-{4-[5-(methylamino)-1,3,4-thiadiazol-2-yl]phenyl}phthalazin-1-amine

MS (EI) for C₂₅H₁₆F₆N₆S: 547 (MH⁺).

### 1-[5-(4-{[3,5-Bis(trifluoromethyl)phenyl]amino}phthalazin-1-yl)-2-thienyl]ethanone

MS (EI) for C₂₂H₁₃F₆N₃OS: 482 (MH⁺).

### Methyl 5-(4-{[3,5-bis(trifluoromethyl)phenyl]amino}phthalazin-1-yl)thiophene-2-carboxylate

MS (EI) for C₂₂H₁₃F₆N₃O₂S: 498 (MH⁺).

### 1-[3-(4-{[3,5-Bis(trifluoromethyl)phenyl]amino}phthalazin-1-yl)phenyl]ethanone

MS (EI) for C₂₄H₁₅F₆N₃O: 476 (MH⁺).

### 1-[4-(4-{[3,5-Bis(trifluoromethyl)phenyl]amino}phthalazin-1-yl)phenyl]imidazolidin-2-one

Using 1-[4-(4,4,5,5-tetramethyl-1,3,2-dioxaborolan-2-yl)phenyl]imidazolidin-2-one (prepared according to the procedure described for Intermediate 3) ¹H NMR (400 MHz, d₆-DMSO): δ 9.84 (s, 1H), 8.79 (s, 2H), 8.64 (d, 1H), 8.15-8.02 (m, 1H), 7.96-7.95 (m, 2H), 7.73 (d, 2H), 7.63-7.60 (m, 3H), 7.06 (s, 1H), 3.91 (t, 2H), 3.42 (t, 2H). MS (EI) for C₂₅H₁₇F₆N₅O: 518 (MH⁺).

### N-[4-(4-{[3,5-Bis(trifluoromethyl)phenyl]amino}phthalazin-1-yl)phenyl]acetamide

MS (EI) for C₂₄H₁₆F₆N₄O: 491 (MH⁺).

### Ethyl 6-(4-{[3,5-bis(trifluoromethyl)phenyl]amino}phthalazin-1-yl)-1H-indazole-3-carboxylate

Using 1-tert-Butyl 3-ethyl 6-(4,4,5,5-tetramethyl-1,3,2-dioxaborolan-2-yl)-1H-indazole-1,3-dicarboxylate (prepared according to the procedure described for Intermediate 12) ¹H NMR (400 MHz, d₆-DMSO): δ 9.92 (s, 1H), 8.81 (s, 2H), 8.68 (d, 1H), 8.21 (d, 1H), 8.09 (t, 1H), 7.98-7.92 (m, 3H), 7.67 (s, 1H), 7.68 (d, 1H), 4.41 (q, 2H), 1.38 (t, 3H). MS (EI) for C₂₆H₁₇F₆N₅O₂: 546 (MH⁺).

### Methyl 4-(4-{[3,5-bis(trifluoromethyl)phenyl]amino}phthalazin-1-yl)-3-fluorobenzoate

¹H NMR (400 MHz, d₆-DMSO): δ 9.98 (s, 1H), 8.80 (s, 2H), 8.69 (d, 1H), 8.10 (t, 1H), 7.98-7.94 (m, 2H), 7.89 (d, 1H), 7.79 (t, 1H), 7.68 (s, 1H), 7.62 (d, 1H), 3.89 (s, 3H). MS (EI) for C₂₄H₁₄F₇N₃O₂: 510 (MH⁺).

### Methyl 4-(4-{[3,5-bis(trifluoromethyl)phenyl]amino}phthalazin-1-yl)-3-chlorobenzoate

¹H NMR (400 MHz, d₆-DMSO): δ 9.97 (s, 1H), 8.82 (s, 2H), 8.70 (d, 1H), 8.12 (s, 1H), 8.08 (d, 2H), 7.93 (t, 1H), 7.73 (d, 1H), 7.69 (s, 1H), 7.44 (d, 1H), 3.90 (s, 3H).

MS (EI) for C₂₄H₁₄ClF₆N₃O₂: 526 (MH⁺), Chlorine isotope pattern.

### Example 2

### Methyl 4-(4-{[3-bromo-5-(trifluoromethyl)phenyl]amino}phthalazin-1-yl)benzoate

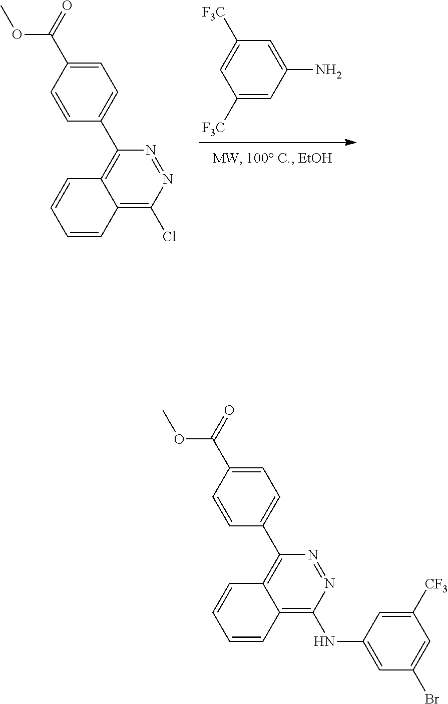 [SMILES: COC(=O)c1ccc(-c2nnc(Cl)c3ccccc23)cc1 ; Nc1cc(C(F)(F)F)cc(C(F)(F)F)c1 ; COC(=O)c1ccc(-c2nnc(Nc3cc(Br)cc(C(F)(F)F)c3)c3ccccc23)cc1]

Methyl 4-(4-chlorophthalazin-1-yl)benzoate (prepared according to the procedure for Intermediate 2) (120 mg, 0.401 mmol) and 3-bromo-5-(trifluoromethyl)aniline (144 mg, 0.602 mmol) were suspended in ethanol (4.0 mL) and the mixture was irradiated with microwaves at 100° C. for 30 minutes. The mixture was concentrated and the residue was washed with a minimal amount of dichloromethane and diethyl ether to give methyl 4-(4-{[3-bromo-5-(trifluoromethyl)phenyl]amino}phthalazin-1-yl)benzoate (129 mg, 64% yield) as a yellow solid. ¹H NMR (600 MHz, d₆-DMSO): δ 8.85 (d, 1H), 8.54 (s, 1H), 8.34 (s, 1H), 8.21-8.13 (m, 3H), 8.07 (t, 1H), 7.95 (d, 1H), 7.85 (d, 2H), 7.69 (s, 1H), 3.89 (d, 3H).

MS (EI) for C₂₃H₁₅BrF₃N₃O₂: 502 (MH⁺), Bromine isotope pattern.

The following compounds were synthesized in an analogous fashion to the compound described above.

### Methyl 4-(4-{[3-methyl-5-(trifluoromethyl)phenyl]amino}phthalazin-1-yl)benzoate

¹H NMR (400 MHz, d₆-DMSO): δ 8.99 (d, 1H), 8.27 (t, 1H), 8.21 (d, 2H), 8.16 (t, 1H), 8.05 (s, 1H), 8.00 (d, 1H), 7.92 (s, 1H), 7.88 (d, 2H), 7.48 (s, 1H), 6.84-6.80 (m, 1H), 3.93 (s, 3H), 2.47 (s, 3H). MS (EI) for C₂₄H₁₈F₃N₃O₂: 438 (MH⁺).

### Methyl 4-{4-[(3-ethylphenyl)amino]phthalazin-1-yl}benzoate

¹H NMR (600 MHz, d₆-DMSO): δ 9.31 (s, 1H), 8.70 (d, 1H), 8.16 (d, 2H), 8.05 (t, 1H), 7.96 (t, 1H), 7.89 (d, 1H), 7.82 (dd, 4H), 7.29 (t, 1H), 6.93 (d, 1H), 3.93 (s, 3H), 2.65 (q, 2H), 1.24 (t, 3H). MS (EI) for C₂₄H₂₁N₃O₂: 384 (MH⁺).

### Methyl 4-(4-{[3-chloro-5-(trifluoromethyl)phenyl]amino}phthalazin-1-yl)benzoate

¹H NMR (600 MHz, d₆-DMSO): δ 8.91 (d, 1H), 8.36 (s, 1H), 8.26 (s, 1H), 8.21-8.12 (m, 3H), 8.05 (t, 1H), 7.93 (d, 1H), 7.82 (d, 2H), 7.57 (s, 1H), 3.86 (s, 3H). MS (EI) for C₂₃H₁₅ClF₃N₃O₂: 458 (MH⁺), Chorine isotope pattern.

### Methyl 4-(4-{[3-(dimethylamino)phenyl]amino}phthalazin-1-yl)benzoate

¹H NMR (400 MHz, d₆-DMSO): δ 9.15 (s, 1H), 8.69 (d, 1H), 8.15 (d, 2H), 8.03 (t, 1H), 7.94 (t, 1H), 7.83 (d, 1H), 7.76 (d, 2H), 7.39-7.37 (m, 2H), 7.17 (t, 1H), 6.49-6.46 (m, 1H), 3.92 (s, 3H), 2.50 (s, 6H). MS (EI) for C₂₄H₂₂N₄O₂: 399 (MH⁺).

### Methyl 4-(4-{[3-(trifluoromethyl)phenyl]amino}phthalazin-1-yl)benzoate

¹H NMR (600 MHz, d₆-DMSO): δ 9.03 (dd, 1H), 8.23 (dd, 2H), 8.17 (d, 2H), 8.15-8.10 (m, 1H), 8.09-8.03 (m, 1H), 7.96 (d, 1H), 7.88-7.81 (m, 2H), 7.70 (s, 1H), 7.64-7.56 (m, 1H), 3.89 (s, 3H). MS (EI) for C₂₃H₁₆F₃N₃O₂: 424 (MH⁺).

### Methyl 4-(4-{[3-(4-methylpiperazin-1-yl)-5-(trifluoromethyl)phenyl]amino}phthalazin-1-yl)benzoate

¹H NMR (600 MHz, d₆-DMSO): δ 9.45 (s, 1H), 8.71 (d, 1H), 8.17 (d, 2H), 8.08 (t, 1H), 7.98 (m, 2H), 7.86 (m, 4H), 6.90 (s, 1H), 3.92 (s, 3H), 3.28 (m, 8H), 2.08 (s, 3H). MS (EI) for C₂₈H₂₆F₃N₅O₂: 522 (MH⁺).

### Methyl 4-[4-({3-[(trifluoromethyl)oxy]phenyl}amino)phthalazin-1-yl]benzoate

¹H NMR (400 MHz, d₆-DMSO): δ 9.02 (d, 1H), 8.29 (t, 1H), 8.24 (d, 2H), 8.16 (t, 1H), 8.00 (d, 1H), 7.96 (br-s, 1H), 7.89 (d, 2H), 7.84 (d, 1H), 7.63 (t, 1H), 7.27 (d, 1H), 3.94 (s, 3H). MS (EI) for C₂₃H₁₆F₃N₃O₂: 440 (MH⁺).

### 4-{4-[(3-bromophenyl)amino]phthalazin-1-yl}benzoate

¹H NMR (400 MHz, d₆-DMSO): δ 8.98-8.96 (m, 1H), 8.26 (t, 1H), 8.22-8.20 (m, 2H), 8.17-8.12 (m, 2H), 7.99 (d, 1H), 7.89-7.87 (m, 2H), 7.78-7.77 (m, 1H), 7.49-7.47 (m, 2H), 3.92 (s, 3H). MS (EI) for C₂₂H₁₆BrN₃O₂: 434 (MH⁺), Bromine isotope pattern.

### Methyl 4-{4-[(3-chlorophenyl)amino]phthalazin-1-yl}benzoate

¹H NMR (400 MHz, d₆-DMSO): δ 9.00-8.99 (m, 1H), 8.26 (t, 1H), 8.21 (d, 2H), 8.15 (t, 1H), 8.01-7.99 (m, 2H), 7.88 (d, 2H), 7.73 (d, 1H), 7.54 (t, 1H), 7.35 (d, 1H), 3.94 (s, 3H). MS (EI) for C₂₂H₁₆ClN₃O₂: 390 (MH⁺), Chlorine isotope pattern.

### Methyl 4-(4-{[3-(1-methylethyl)phenyl]amino}phthalazin-1-yl)benzoate

¹H NMR (400 MHz, d₆-DMSO): δ 8.97 (d, 1H), 8.25 (t, 1H), 8.21-8.16 (m, 3H), 7.97 (d, 1H), 7.84 (d, 2H), 7.56-7.52 (m, 2H), 7.47 (t, 1H), 7.29-7.27 (m, 1H), 3.93 (s, 3H), 2.97 (quint, 1H), 1.26 (d, 6H). MS (EI) for C₂₅H₂₃N₃O₂: 398 (MH⁺).

### Methyl 4-(4-{[4-chloro-3-(trifluoromethyl)phenyl]amino}phthalazin-1-yl)benzoate

¹H NMR (400 MHz, d₆-DMSO): δ 8.92 (d, 1H), 8.46 (s, 1H), 8.23 (m, 4H), 8.12 (t, 1H), 7.99 (d, 1H), 7.87 (d, 2H), 7.81 (d, 1H), 3.93 (s, 3H). MS (EI) for C₂₃H₁₅ClF₃N₃O₂: 458 (MH⁺), Chlorine isotope pattern.

### Methyl 4-(4-{[3-fluoro-5-(trifluoromethyl)phenyl]amino}phthalazin-1-yl)benzoate

¹H NMR (400 MHz, d₆-DMSO): δ 8.89 (d, 1H), 8.25-8.20 (m, 5H), 8.11 (t, 1H), 7.99 (d, 1H), 7.89 (d, 2H), 7.45 (d, 1H), 3.92 (s, 3H). MS (EI) for C₂₃H₁₅F₄N₃O₂: 442 (MH⁺).

### Methyl 4-(4-{[3-{[2-(dimethylamino)ethyl]oxy}-5-(trifluoromethyl)phenyl]amino}phthalazin-1-yl)benzoate hydrochloride salt

Using 3-[2-(dimethylamino)ethoxy]-5-(trifluoromethyl)aniline hydrochloride salt (the hydrochloride salt of the aniline prepared according to the procedure for Intermediate 5). ¹H NMR (400 MHz, d₆-DMSO): δ 9.61 (s, 1H), 8.74 (d, 1H), 8.18 (d, 2H), 8.09 (m, 3H), 8.00 (t, 1H), 7.92 (d, 1H), 7.88 (d, 2H), 6.93 (s, 1H), 4.16 (t, 2H), 3.93 (s, 3H), 2.68 (t, 2H), 2.21 (s, 6H). MS (EI) for C₂₂H₂₅F₃N₄O₃: 511 (MH⁺).

### Methyl 4-[4-({3-[3-(dimethylamino)propyl]-5-(trifluoromethyl)phenyl}amino)phthalazin-1-yl]benzoate hydrochloride salt

Using 3-[3-(dimethylamino)propyl]-5-(trifluoromethyl)aniline hydrochloride salt (the hydrochloride salt of the aniline prepared according to the procedure for Intermediate 6). ¹H NMR (400 MHz, d₆-DMSO): δ 9.64 (s, 1H), 8.74 (d, J=8.13 Hz, 1H), 8.36 (s, 1H), 8.24-8.14 (m, 3H), 8.09 (t, 1H), 7.99 (t, 1H), 7.93 (d, 1H), 7.86 (d, J=7.97 Hz, 2H), 7.27 (s, 1H), 3.93 (s, 3H), 2.76 (t, 2H), 2.62 (m, 3H), 2.42 (s, 6H), 1.88 (m, 2H). MS (EI) for C₂₈H₂₂F₃N₄O₂: 509 (MH⁺).

### Methyl 4-{4-[(3-cyanophenyl)amino]phthalazin-1-yl}benzoate

¹H NMR (400 MHz, d₆-DMSO): δ 8.97 (d, 1H), 8.39 (s, 1H), 8.2-8.20 (m, 3H), 8.14 (m, 3H), 7.99 (d, 1H), 7.89 (d, 2H), 7.69 (m, 2H), 3.93 (s, 3H). MS (EI) for C₂₃H₁₆N₄O₂: 381 (MH⁺).

### Methyl 4-(4-{[4-(methyloxy)-3-(trifluoromethyl)phenyl]amino}phthalazin-1-yl)benzoate

¹H NMR (400 MHz, d₆-DMSO): δ 9.45 (s, 1H), 8.66 (d, 1H), 8.30 (s, 1H), 8.21 (d, 1H), 8.14 (d, 2H), 8.06 (t, 1H), 7.96 (t, 1H), 7.88 (d, 1H), 7.82 (d, 2H), 7.32 (d, 1H), 3.91 (d, 6H). MS (EI) for C₂₄H₁₈F₃N₃O₃: 454 (MH⁺).

### Methyl 4-(4-{[4-fluoro-3-(trifluoromethyl)phenyl]amino}phthalazin-1-yl)benzoate

¹H NMR (400 MHz, d₆-DMSO): δ 9.00 (d, 1H), 8.28 (m, 2H), 8.20 (d, 2H), 8.13 (m, 2H), 7.98 (d, 1H), 7.86 (d, 2H), 7.68 (t, 1H), 3.92 (s, 3H). MS (EI) for C₂₃H₁₅F₄N₃O₂: 442 (MH⁺).

### Methyl 4-{4-[(3-bromo-5-methylphenyl)amino]phthalazin-1-yl}benzoate

¹H NMR (400 MHz, d₆-DMSO): δ 8.82 (d, 1H), 8.15 (m, 3H), 8.05 (t, 1H), 7.92 (m, 2H), 7.82 (d, 2H), 7.61 (s, 1H), 7.21 (s, 1H), 3.88 (s, 3H), 2.32 (s, 3H). MS (EI) for C₂₃H₁₈BrN₃O₂: 448 (MH⁺), Bromine isotope pattern.

### Methyl 4-[4-({3-[(dimethylamino)methyl]-5-(trifluoromethyl)phenyl}amino)phthalazin-1-yl]benzoate

Using 3-[(dimethylamino)methyl]-5-(trifluoromethyl)aniline hydrochloride salt (the hydrochloride salt of the aniline prepared according to the procedure for Intermediate 8). ¹H NMR (400 MHz, d₆-DMSO): δ 9.65 (s, 1H), 8.74 (d, 1H), 8.48 (s, 1H), 8.26 (s, 1H), 8.16 (d, 2H), 8.09 (t, 1H), 7.99 (t, 1H), 7.87 (d, 1H), 7.86 (d, 2H), 7.30 (s, 1H), 3.93 (s, 3H), 3.54 (s, 2H), 2.23 (s, 6H). MS (EI) for C₂₆H₂₃F₃N₄O₂: 481 (MH⁺).

### Methyl 4-(4-{[3-(1,1-dimethylethyl)phenyl]amino}phthalazin-1-yl)benzoate

¹H NMR (400 MHz, d₆-DMSO): δ 8.72 (d, 1H), 8.16 (d, 2H), 8.06 (t, 1H), 7.98-7.83 (m, 6H), 7.33 (t, 1H), 7.12 (d, 1H), 3.92 (s, 3H), 1.34 (s, 9H). MS (EI) for C₂₆H₂₅N₃O₂: 412 (MH⁺).

### Methyl 3-[(4-{4-[(methyloxy)carbonyl]phenyl}phthalazin-1-yl)amino]-5-(trifluoromethyl)benzoate

Using 3-amino-5-(trifluoromethyl)benzoate (prepared according to the procedure for Intermediate 9). ¹H NMR (400 MHz, d₆-DMSO): δ 9.92 (s, 1H), 8.97 (s, 1H), 8.89 (s, 1H), 8.76 (d, 1H), 8.18 (d, 2H), 8.13 (t, 1H), 8.02 (t, 1H), 7.94 (d, 1H), 7.89-7.85 (m, 3H), 3.94 (d, 6H). MS (EI) for C₂₅H₁₈F₃N₃O₄: 482 (MH⁺).

### Methyl 4-[4-({3-[2-(dimethylamino)ethyl]-5-(trifluoromethyl)phenyl}amino)phthalazin-1-yl]benzoate

Using 3-[2-(dimethylamino)ethyl]-5-(trifluoromethyl)aniline hydrochloride salt (prepared according to the procedure for Intermediate 10). ¹H NMR (400 MHz, d₆-DMSO): δ 9.56 (s, 1H), 8.67 (d, 1H), 8.34 (s, 1H), 8.12-8.10 (m, 3H), 8.04 (t, 1H), 7.94 (t, 1H), 7.86 (d, 1H), 7.80 (d, 2H), 7.23 (s, 1H), 3.88 (s, 3H), 2.81 (t, 2H), 2.65-2.60 (m, 2H), 2.23 (s, 6H). MS (EI) for C₂₇H₂₅F₃N₄O₂: 495 (MH⁺).

### Methyl 4-(4-{[3-(1-methylpiperidin-4-yl)-5-(trifluoromethyl)phenyl]amino}phthalazin-1-yl)benzoate

Using 3-(1-methylpiperidin-4-yl)-5-(trifluoromethyl)aniline hydrochloride salt (the hydrochloride salt of the aniline prepared according to the procedure for Intermediate 11). ¹H NMR (400 MHz, d₆-DMSO): δ 9.66 (s, 1H), 8.76 (d, 1H), 8.41 (s, 1H), 8.26 (s, 1H), 8.17 (d, 2H), 8.07 (t, 1H), 7.99 (t, 1H), 7.87 (m, 3H), 7.25 (s, 1H), 3.93 (s, 3H), 3.08 (br s, 2H), 2.71 (m, 1H), 2.39 (m, 5H), 1.82 (m, 4H). MS (EI) for C₂₉H₂₇F₃N₄O₂: 521 (MH⁺).

### Example 3

### 4-(4-{[3,5-Bis(trifluoromethyl)phenyl]amino}phthalazin-1-yl)benzoic acid and 4-(4-{[3,5-Bis(trifluoromethyl)phenyl]amino}phthalazin-1-yl)-N-(2-morpholin-4-ylethyl)benzamide formic acid salt

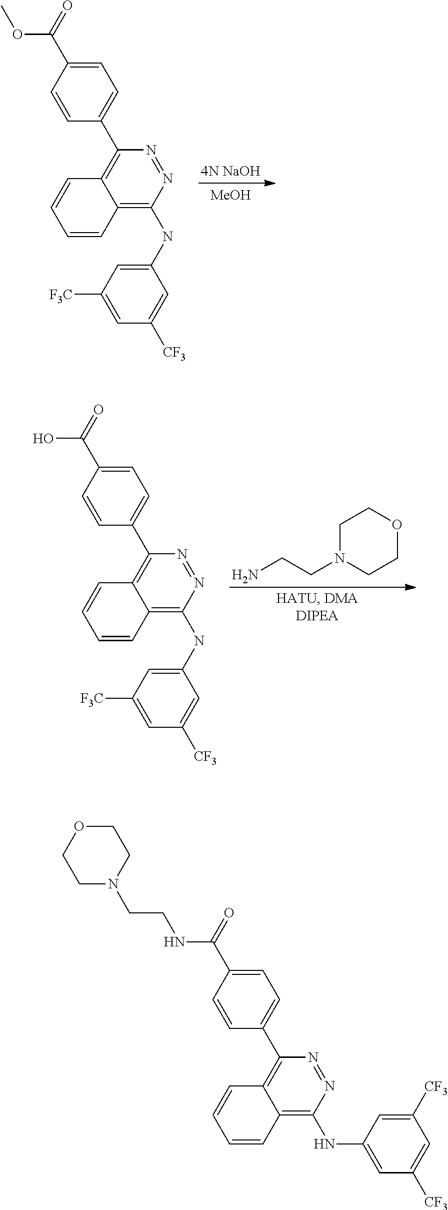

### 4-(4-{[3,5-Bis(trifluoromethyl)phenyl]amino}phthalazin-1-yl)benzoic acid

A solution of methyl 4-(4-{[3,5-bis(trifluoromethyl)phenyl]amino}phthalazin-1-yl)benzoate (prepared according to the procedure for Example 1) (1.5 g, 3.053 mol) in methanol (30 mL) and 4N aqueous sodium hydroxide (3.3 mL) was heated at reflux for 2 h. After cooling to room temperature, the volatiles were removed in vacuo. The residue was dissolved in water and was washed with diethyl ether (2×). The aqueous layer was acidified and the resulting precipitate was collected and dried under vacuum to give 4-(4-{[3,5-bis(trifluoromethyl)phenyl]amino}phthalazin-1-yl)benzoic acid (1.4 g, 96% yield) as a solid. ¹H NMR (400 MHz, d₆-DMSO); δ 10.0 (s, 1H), 8.86 (s, 2H), 8.75 (d, 1H), 8.16 (m, 2H), 8.02 (t, 1H), 7.96 (m, 1H), 7.86 (d, 2H), 7.71 (s, 1H), 7.56 (m, 1H). MS (EI) for C₂₃H₁₃F₆N₃O₂: 478 (MH⁺).

### 4-(4-{[3,5-Bis(trifluoromethyl)phenyl]amino}phthalazin-1-yl)-N-(2-morpholin-4-ylethyl)benzamide formic acid salt

To 4-(4-{[3,5-bis(trifluoromethyl)phenyl]amino}phthalazin-1-yl)benzoic acid (30 mg, 0.063 mmol) and O-(7-azabenzotriazol-1-yl)-N,N,N′,N′-tetramethyluronium hexafluorophosphate (HATU) (24 mg, 0.063 mmol) in N,N-dimethylacetamide (0.3 mL) was added diisopropylethylamine (0.025 mL, 0.144 mmol) and 4-(2-aminoethyl)morpholine (0.009 mL, 0.069 mmol) and the mixture was stirred at room temperature for 3 h and then was purified by preparative HPLC to afford 4-(4-{[3,5-bis(trifluoromethyl)phenyl]amino}phthalazin-1-yl)-N-(2-morpholin-4-ylethyl)benzamide formic acid salt (23 mg, 0.036 mmol, 57% yield) as a pale yellow solid. ¹H NMR (400 MHz, d₆-DMSO) δ 9.90 (s, 1H), 8.78 (s, 2H), 8.66 (d, 1H), 8.54 (t, 1H), 8.10-8.04 (m, 1H), 8.01-7.93 (m, 3H), 7.88 (d, 1H), 7.77-7.72 (m, 2H), 7.65 (s, 1H), 3.53 (t, 4H), 3.39 (q, 2H), 2.44 (q, 2H), 2.42-2.36 (m, 4H). MS (EI) for C₂₉H₂₅F₆N₅O₂: 590 (MH⁺).

### Example 4

### Methyl 4-(4-{[3-{[2-(dimethylamino)ethyl]amino}-5-(trifluoromethyl)phenyl]amino}phthalazin-1-yl)benzoate

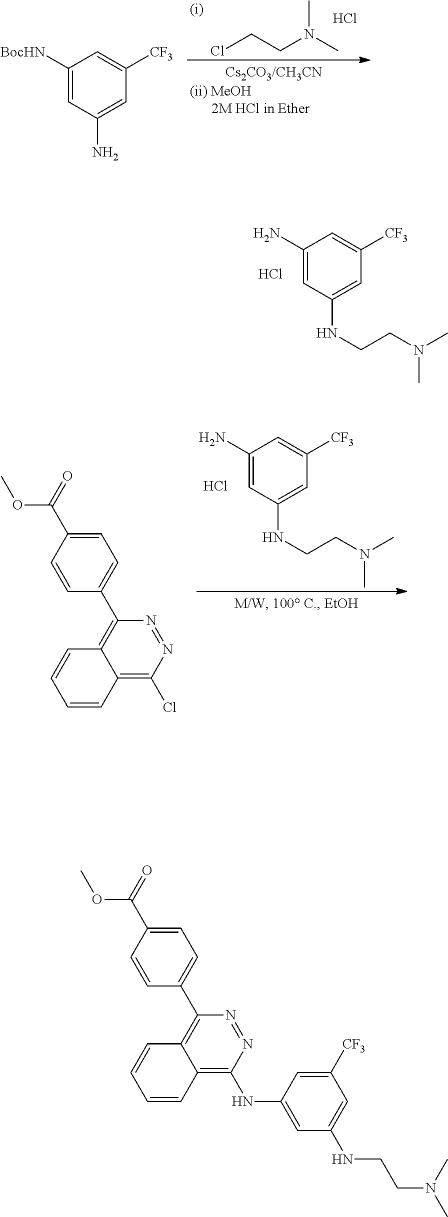

### N-[2-(Dimethylamino)ethyl]-5-(trifluoromethyl)benzene-1,3-diamine hydrochloride salt

To a solution of tert-butyl [3-amino-5-(trifluoromethyl)phenyl]carbamate (400 mg, 1.45 mmol) in acetonitrile (20 mL) was added cesium carbonate (1.42 g, 4.34 mmol) and 2-(dimethylamino)ethyl chloride hydrochloride (313 mg, 2.17 mmol) with constant stirring. The resulting mixture was heated at 100° C. for 15 h. and then was filtered through a Celite pad. The filtrate was evaporated to give a residue. Silica gel column chromatography (dichloromethane:methanol=10:1, R_{f}=0.3 in dichloromethane; methanol=4:1) gave a brown oil which was dissolved in methanol (10 mL). 2M Hydrochloric acid in diethyl ether (4.8 mL, 9.44 mmol) was carefully added dropwise. The mixture was stirred at room temperature for 15 h and then was concentrated to give N-[2-(dimethylamino)ethyl]-5-(trifluoromethyl)benzene-1,3-diamine hydrochloride salt (134 mg, 0.473 mmol, 33% yield) as a pale yellow solid.

### Methyl 4-(4-{[3-{[2-(dimethylamino)ethyl]amino}-5-(trifluoromethyl)phenyl]amino}phthalazin-1-yl)benzoate

Methyl 4-(4-chlorophthalazin-1-yl)benzoate (prepared according to the procedure for Intermediate 2) (65 mg, 0.167 mmol) and N-[2-(dimethylamino)ethyl]-5-(trifluoromethyl)benzene-1,3-diamine hydrochloride salt (79 mg, 0.328 mmol) were suspended in ethanol (2.0 mL) and the mixture was irradiated with microwaves at 100° C. for 20 minutes. The mixture was concentrated and then the residue was purified by silica gel column chromatography (eluted with hexanes:ethyl acetate=3:1, R_{f}=0.1 in hexanes:ethyl acetate=3:1) to give the crude product which was washed with a minimal amount of dichloromethane and diethyl ether to afford methyl 4-(4-{[3-{[2-(dimethylamino)ethyl]amino}-5-(trifluoromethyl)phenyl]amino}phthalazin-1-yl)benzoate as a colorless solid (20 mg, 0.043 mmol, 20% yield). ¹H NMR (400 MHz, d₆-DMSO): δ 9.38 (s, 1H), 8.71 (d, 1H), 8.16 (d, 2H), 8.07 (t, 1H), 7.97 (t, 1H), 7.90 (d, 1H), 7.85 (d, 2H), 7.60 (d, 2H), 6.60 (NH, s, 1H), 6.05 (NH, br s, 1H), 3.93 (s, 3H), 3.21 (s, 2H), 2.58 (s, 2H), 2.28 (s, 6H). MS (EI) for C₂₂H₂₆F₃N₅O₂: 510 (MH⁺).

### Example 5

### Methyl 4-(4-{[3-ethyl-5-(trifluoromethyl)phenyl]amino}phthalazin-1-yl)benzoate

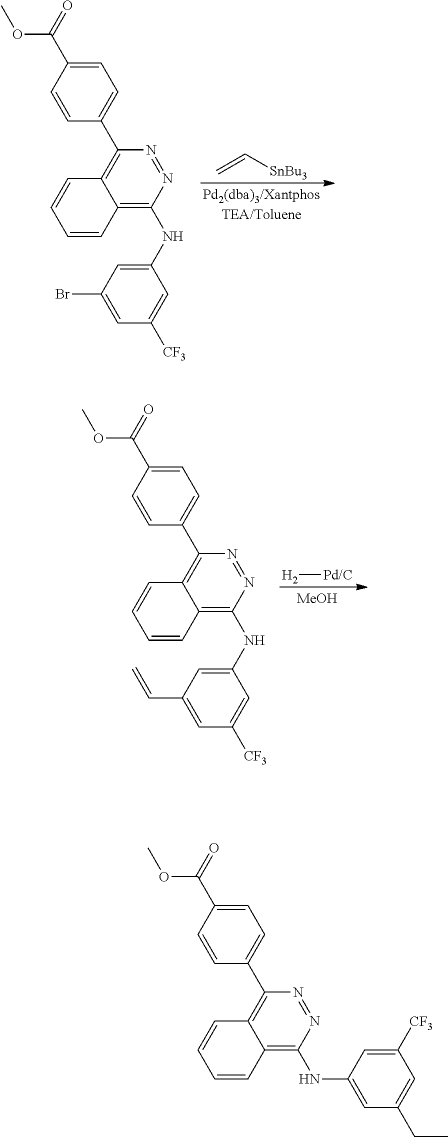

### Methyl 4-(4-{[3-(trifluoromethyl)-5-vinylphenyl]amino}phthalazin-1-yl)benzoate

Methyl 4-(4-{[3-bromo-5-(trifluoromethyl)phenyl]amino}phthalazin-1-yl)benzoate (prepared according to the procedure for Example 2) (120 mg, 0.238 mmol), tributyl(vinyl)tin (0.138 mL, 0.476 mmol), Pd₂(dba)₃ (10.8 mg, 0.0119 mmol), Xantphos (27.5 mg, 0.0476 mmol) and triethylamine (0.1 mL, 0.714 mmol) were suspended in toluene (5 mL) under nitrogen atmosphere and the mixture was stirred in a sealed tube at 80° C. overnight. After cooling to room temperature, the mixture was filtered through a Celite pad and the filtrate was concentrated. The residue was stirred with 1,8-diazabicyclo[5.4.0]undec-7-ene (0.106 mL, 0.714 mmol) and iodine in dichloromethane (0.1 M; 0.714 mmol) for 1 h. Volatiles were removed and the residue was purified by column chromatography (eluted with ethyl acetate:hexanes=1:4 to 1:2) to give methyl 4-(4-{[3-(trifluoromethyl)-5-vinylphenyl]amino}phthalazin-1-yl)benzoate (43 mg, 40% yield) as a yellow solid. ¹H NMR (400 MHz, CDCl₃+CD₃OD): δ 8.57 (d, 1H), 8.23 (m, 3H), 8.04 (m, 1H), 7.93 (m, 2H), 7.78 (d, 2H), 7.39 (s, 1H), 6.82 (q, 1H), 5.95 (d, 1H), 5.40 (d, 1H), 3.99 (s, 3H).

### Methyl 4-(4-{[3-ethyl-5-(trifluoromethyl)phenyl]amino}phthalazin-1-yl)benzoate

A mixture of methyl 4-(4-{[3-(trifluoromethyl)-5-vinylphenyl]amino}phthalazin-1-yl)benzoate (40 mg, 0.089 mmol) and 10% Pd on C (4 mg) in methanol (10 mL) was stirred under hydrogen (45 psi) for 2 h. The reaction mixture was filtered through a Celite pad and the filtrate was evaporated. The residue was purified by column chromatography (eluted with dichloromethane:methanol=20:1) to give methyl 4-(4-{[3-ethyl-5-(trifluoromethyl)phenyl]amino}phthalazin-1-yl)benzoate (40 mg, 99%, 0.0886 mmol) as a colorless solid. ¹H NMR (400 MHz, d₆-DMSO): δ 9.58 (s, 1H), 8.72 (d, 1H), 8.37 (s, 1H), 8.17 (m, 3H), 8.09 (t, 1H), 7.98 (t, 1H), 7.91 (d, 1H), 7.85 (d, 2H), 7.24 (s, 1H), 3.92 (s, 3H), 2.75 (q, 2H), 1.26 (t, 3H). MS (EI) for C₂₅H₂₀F₃N₃O₂: 452 (MH⁺).

### Example 6

### N-[3,5-Bis(trifluoromethyl)phenyl]-4-(3-methyl-1H-indazol-6-yl)phthalazin-1-amine

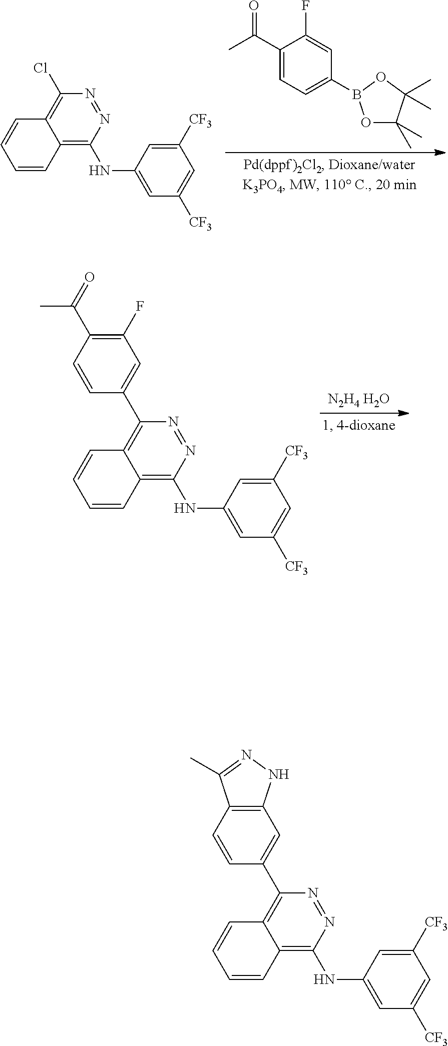 [SMILES: FC(F)(F)c1cc(Nc2nnc(Cl)c3ccccc23)cc(C(F)(F)F)c1 ; CC(=O)c1ccc(B2OC(C)(C)C(C)(C)O2)cc1F ; CC(=O)c1ccc(-c2nnc(Nc3cc(C(F)(F)F)cc(C(F)(F)F)c3)c3ccccc23)cc1F ; "N2H4 H2O" ; Cc1n[nH]c2cc(-c3nnc(Nc4cc(C(F)(F)F)cc(C(F)(F)F)c4)c4ccccc34)ccc12]

### 1-[4-(4-{[3,5-Bis(trifluoromethyl)phenyl]amino}phthalazin-1-yl)-2-fluorophenyl]ethanone

N-[3,5-Bis(trifluoromethyl)phenyl]-4-chlorophthalazin-1-amine (prepared according to the procedure for Intermediate 1) (150 mg, 0.383 mmol), 1-[2-fluoro-4-(4,4,5,5-tetramethyl-1,3,2-dioxaborolan-2-yl)phenyl]ethanone (prepared according to the procedure for Intermediate 4) (151 mg, 0.574 mmol), Pd(dppf)₂Cl₂ dichloromethane adduct (31 mg, 0.0383 mmol) and potassium phosphate tribasic (243 mg, 1.15 mmol) were suspended in 1,4-dioxane (4 mL) and water (0.4 mL) and the mixture was irradiated with microwaves at 110° C. for 20 minutes. The mixture was treated with 1N aqueous sodium hydroxide (10 mL) and extracted with ethyl acetate (20 mL). The organic layer was washed 1N aqueous sodium hydroxide (2×10 mL), was dried and was concentrated. The residue was purified by column chromatography (eluted with ethyl acetate:hexanes=1:4) to give 1-[4-(4-{[3,5-bis(trifluoromethyl)phenyl]amino}phthalazin-1-yl)-2-fluorophenyl]ethanone (135 mg, 71% yield) as an ivory solid. ¹H NMR (400 MHz, d₆-DMSO): δ 9.99 (s, 1H), 8.84 (s, 2H), 8.72 (d, 1H), 8.15 (t, 1H), 8.03 (m, 2H), 7.96 (d, 1H), 7.72 (m, 3H), 2.68 (d, 3H).

### N-[3,5-Bis(trifluoromethyl)phenyl]-4-(3-methyl-1H-indazol-6-yl)phthalazin-1-amine

1-[4-(4-{[3,5-Bis(trifluoromethyl)phenyl]amino}phthalazin-1-yl)-2-fluorophenyl]ethanone (130 mg, 0.263 mmol) and hydrazine hydrate (0.13 mL, 2.63 mmol) were dissolved in 1,4-dioxane (10 mL). The mixture was heated at reflux overnight and then was cooled to room temperature. The mixture was extracted with ethyl acetate and water. The organic layer was washed, was dried over magnesium sulfate and was concentrated. The residue was purified by column chromatography (eluted with ethyl acetate:hexanes=1:2) to give N-[3,5-bis(trifluoromethyl)phenyl]-4-(3-methyl-1H-indazol-6-yl)phthalazin-1-amine (32 mg, 0.065 mmol, 25% yield) as an ivory solid. ¹H NMR (400 MHz, d₆-DMSO): δ 12.86 (s, 1H), 9.93 (s, 1H), 8.86 (s, 2H), 8.72 (d, 1H), 8.12 (t, 1H), 8.00 (m, 2H), 7.89 (d, 1H), 7.73 (d, 2H), 7.38 (d, 1H), 2.54 (s, 3H). MS (EI) for C₂₄H₁₅F₆N₅: 488 (MH⁺).

The following compounds were synthesized in an analogous fashion to the compound described above.

### 4-(3-Amino-1H-indazol-6-yl)-N-[3,5-bis(trifluoromethyl)phenyl]phthalazin-1-amine

MS (EI) for C₂₃H₁₄F₆N₆: 489 (MH⁺).

### N-[3,5-Bis(trifluoromethyl)phenyl]-4-(3-ethyl-1H-indazol-6-yl)phthalazin-1-amine

Using 1-[2-fluoro-4-(4,4,5,5-tetramethyl-1,3,2-dioxaborolan-2-yl)phenyl]propan-1-one (prepared according to the procedure for Intermediate 13). ¹H NMR (400 MHz, d₆-DMSO): δ 9.94 (s, 1H), 8.86-8.81 (m, 2H), 8.71 (d, 1H), 8.12-8.10 (m, 1H), 8.00-8.98 (m, 2H), 7.92 (d, 1H), 7.74-7.70 (m, 2H), 7.36 (d, 1H), 3.01 (q, 2H), 1.38 (t, 3H). MS (EI) for C₂₅H₁₇F₆N₅: 502 (MH⁺).

### Example 7

### Methyl 4-(4-{[3-{[3-(dimethylamino)propyl]amino}-5-(trifluoromethyl)phenyl]amino}phthalazin-1-yl)benzoate

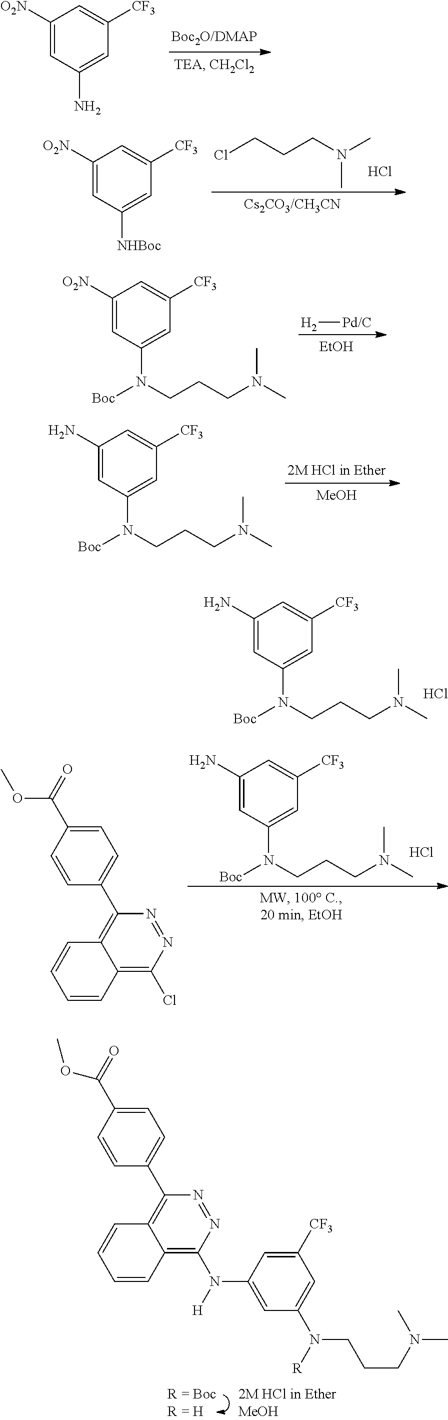

### tert-Butyl [3-nitro-5-(trifluoromethyl)phenyl]carbamate

To a solution of 3-amino-5-nitrobenzotrifluoride (300 mg, 1.46 mmol) in dichloromethane (10 mL) was added triethylamine (0.2 ml, 1.46 mmol) and 4-(dimethylamino)pyridine (178 mg, 1.46 mmol) at 0° C. Di-tert-butyl dicarbonate (476 mg, 2.183 mmol) was added to the solution in small portions. The resulting solution was stirred for 15 h, was washed with water, saturated sodium bicarbonate solution and brine, was dried over magnesium sulfate and was evaporated to give a residue. Silica gel column chromatography (eluted with hexanes:ethyl acetate=2:1, R_{f}=0.6 in hexanes:ethyl acetate=2:1) gave tert-butyl [3-nitro-5-(trifluoromethyl)phenyl]carbamate (430 mg, 1.404 mmol, 97% yield) as a yellow oil. ¹H NMR (400 MHz, CDCl₃): δ 8.45 (s, 1H), 8.27 (s, 1H), 7.79 (s, 1H), 1.48 (s, 9H).

### tert Butyl [3-(dimethylamino)propyl][3-nitro-5-(trifluoromethyl)phenyl]carbamate

To a solution of tert-butyl [3-nitro-5-(trifluoromethyl)phenyl]carbamate (426 mg, 1.391 mmol) in acetonitrile (10 mL) was added cesium carbonate (1.37 g, 4.201 mmol) and 3-dimethylaminopropyl chloride hydrochloride (330 mg, 2.087 mmol) with stirring. The resulting mixture was heated at 100° C. for 4 h. The mixture was filtered through a Celite pad and the filtrate was evaporated. Silica gel column chromatography of the residue (eluted with dichloromethane:methanol=10:1 to 5:1, R_{f}=0.3 in dichloromethane:methanol=4:1) gave tert-butyl [3-(dimethylamino)propyl][3-nitro-5-(trifluoromethyl)phenyl]carbamate (121 mg, 0.309 mmol, 22% yield) as reddish oil. ¹H NMR (400 MHz, CDCl₃): δ 8.39 (s, 1H), 8.26 (s, 1H), 7.94 (s, 1H), 3.80 (t, 2H), 2.31 (t, 2H), 1.80 (q, 2H), 1.49 (s, 9H).

### tert-Butyl [3-amino-5-(trifluoromethyl)phenyl][3-(dimethylamino)propyl]carbamate

The mixture of tert-butyl [3-(dimethylamino)propyl][3-nitro-5-(trifluoromethyl)phenyl]carbamate (117 mg, 0.299 mmol) and 10% Pd on C (12 mg) in methanol (10 mL) was stirred under hydrogen (60 psi) for 2 h. The reaction mixture was filtered through a Celite pad and the filtrate was evaporated to give tert-butyl [3-amino-5-(trifluoromethyl)phenyl][3-(dimethylamino)propyl]carbamate (105 mg, 97% yield) as brown oil. ¹H NMR (400 MHz, CDCl₃): δ 6.82 (s, 1H), 6.72 (s, 1H), 6.69 (s, 1H), 3.90 (—NH₂, br-s, 2H), 3.63 (t, 2H), 2.29 (t, 2H), 1.73 (q, 2H), 1.44 (s, 9H).

### tert-Butyl [3-amino-5-(trifluoromethyl)phenyl][3-(dimethylamino)propyl]carbamate hydrochloride salt

To a solution of tert-butyl [3-amino-5-(trifluoromethyl)phenyl][3-(dimethylamino)propyl]carbamate (104 mg, 0.287 mmol) in methanol (3 mL) was added 2M hydrochloric acid in diethyl ether (0.29 mL, 0.576 mmol) dropwise. The mixture was stirred at room temperature for 1 h and then it was evaporated to give tert-butyl [3-amino-5-(trifluoromethyl)phenyl][3-(dimethylamino)propyl]carbamate hydrochloride salt (127 mg, quantitative yield) as a pale yellow oily solid.

### Methyl 4-(4-{[3-{(tert-butoxycarbonyl)[3-(dimethylamino)propyl]amino}-5-(trifluoromethyl)phenyl]amino}phthalazin-1-yl)benzoate

tert-Butyl [3-amino-5-(trifluoromethyl)phenyl][3-(dimethylamino)propyl]carbamate hydrochloride salt (120 mg, 0.302 mmol) and methyl 4-(4-chlorophthalazin-1-yl)benzoate (prepared according to the procedure for Intermediate 2) (60 mg, 0.201 mmol) were suspended in ethanol (2.0 mL) and the mixture was irradiated with microwaves at 100° C. for 20 minutes. The mixture was concentrated and the residue was purified by silica gel column chromatography (dichloromethane:methanol=6:1 to 3:1) to give methyl 4-(4-{[3-{(tert-butoxycarbonyl)[3-(dimethylamino)propyl]amino}-5-(trifluoromethyl)phenyl]amino}phthalazin-1-yl)benzoate (50 mg, 0.080 mmol, 40% yield) as a brown oil. ¹H NMR (400 MHz, CDCl₃): δ 8.19 (d, 2H), 7.77-7.66 (m, 8H), 6.51 (s, 1H), 3.97 (s, 3H), 3.20 (t, 2H), 2.44 (t, 2H), 2.27 (d, 6H), 1.79 (q, 2H), 1.44 (s, 9H).

### Methyl 4-(4-{[3-{[3-(dimethylamino)propyl]amino}-5-(trifluoromethyl)phenyl]amino}phthalazin-1-yl)benzoate

To a solution of methyl 4-(4-{[3-{(tert-butoxycarbonyl)[3-(dimethylamino)propyl]amino}-5-(trifluoromethyl)phenyl]amino}phthalazin-1-yl)benzoate (45 mg, 0.072 mmol) in methanol (3 mL) was added 2M hydrochloric acid in diethyl ether (0.072 mL, 0.144 mmol) dropwise. The mixture was stirred at room temperature for 15 h and then it was evaporated to give the product which was washed with a minimal amount of dichloromethane and diethyl ether to afford methyl 4-(4-{[3-{[3-(dimethylamino)propyl]amino}-5-(trifluoromethyl)phenyl]amino}phthalazin-1-yl)benzoate as a brown solid (20 mg, 0.036 mmol, 50% yield). ¹H NMR (400 MHz, d₆-DMSO): δ 9.28 (d, 1H), 8.23 (t, 1H), 8.17 (d, 3H), 7.94 (d, 1H), 7.81 (d, 2H), 7.15 (s, 2H), 6.87 (s, 1H), 3.88 (s, 3H), 3.17-3.11 (m, 4H), 2.68 (d, 6H), 1.95 (q, 2H). MS (EI) for C₂₈H₂₈F₃N₅O₂: 524 (MH⁺).

### Example 8

### 4-(1H-1,2,3-Benzotriazol-6-yl)-N-[3,5-bis(trifluoromethyl)phenyl]phthalazin-1-amine

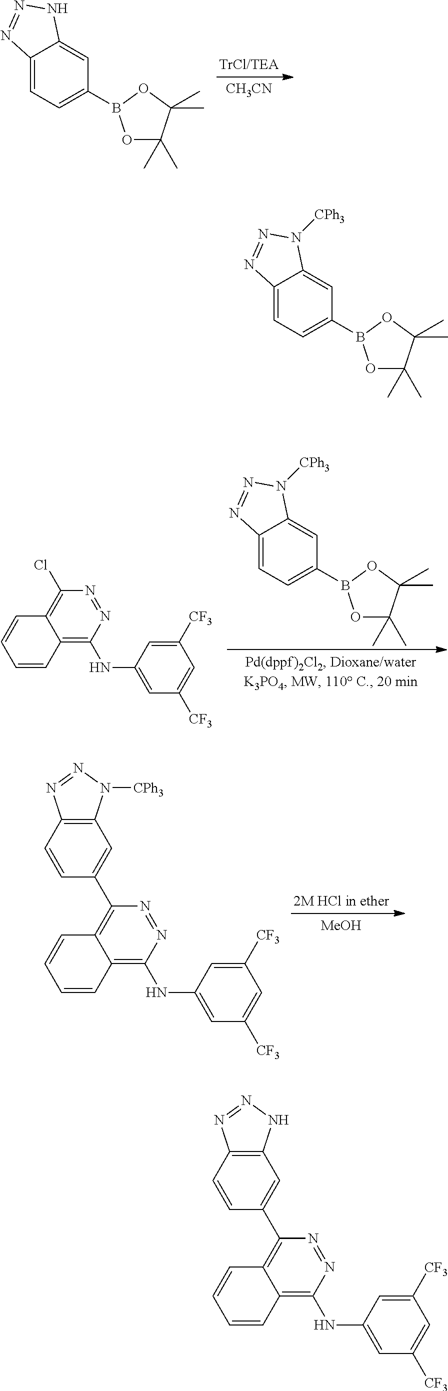

### 6-(4,4,5,5-Tetramethyl-1,3,2-dioxaborolan-2-yl)-1-trityl-1H-benzotriazole

6-(4,4,5,5-Tetramethyl-1,3,2-dioxaborolan-2-yl)-1H-benzotriazole (250 mg, 1.02 mmol) and trityl chloride (425 mg, 1.53 mmol) were dissolved in acetonitrile (10 mL) and triethylamine (0.435 mL, 3.06 mmol) was added. The mixture was stirred overnight and the resulting precipitate was filtered, was washed with hexanes and was dried in vacuo to give 6-(4,4,5,5-tetramethyl-1,3,2-dioxaborolan-2-yl)-1-trityl-1H-benzotriazole (438 mg, 88% yield) as an ivory solid. ¹H NMR (400 MHz, CDCl₃): δ 8.57 (s, 1H), 7.50 (d, 1H), 7.33 (m, 9H), 7.17 (m, 6H), 6.38 (d, 1H), 1.34 (s, 12H).

### N-[3,5-Bis(trifluoromethyl)phenyl]-4-(1-trityl-1H-benzotriazol-6-yl)phthalazin-1-amine

N-[3,5-Bis(trifluoromethyl)phenyl]-4-chlorophthalazin-1-amine (prepared according to the procedure for Intermediate 1) (100 mg, 0.255 mmol), 6-(4,4,5,5-tetramethyl-1,3,2-dioxaborolan-2-yl)-1-trityl-1H-benzotriazole (185 mg, 0.379 mmol), Pd(dppf)₂Cl₂ dichloromethane adduct (20 mg, 0.0245 mmol) and potassium phosphate tribasic (105 mg, 0.494 mmol) were suspended in 1,4-dioxane (4 mL) and water (0.4 mL), and the mixture was irradiated with microwaves at 110° C. for 20 minutes. The mixture was treated with 1N aqueous sodium hydroxide (10 mL) and was extracted with ethyl acetate. The organic layer was washed with 1N aqueous sodium hydroxide (2×10 mL), was dried and was concentrated. The residue was purified by column chromatography (eluted with ethyl acetate:hexanes=1:4) to give N-[3,5-bis(trifluoromethyl)phenyl]-4-(1-trityl-1H-benzotriazol-6-yl)phthalazin-1-amine (140 mg, 77% yield) as a colorless solid. ¹H NMR (400 MHz, d₆-DMSO): δ 9.95 (s, 1H), 8.83 (s, 2H), 8.71 (d, 1H), 8.40 (s, 1H), 8.12 (t, 1H), 7.98 (m, 2H), 7.70 (s, 1H), 7.61 (d, 1H), 7.42 (m, 9H), 7.15 (m, 6H), 6.64 (d, 1H).

### 4-(1H-1,2,3-Benzotriazol-6-yl)-N-[3,5-bis(trifluoromethyl)phenyl]phthalazin-1-amine

N-[3,5-Bis(trifluoromethyl)phenyl]-4-(1-trityl-1H-benzotriazol-6-yl)phthalazin-1-amine (140 mg, 0.195 mmol) was dissolved in methanol (10 mL) and 2M hydrochloric acid in diethyl ether (1.5 mL) was added to the solution. The mixture was stirred for 1 h and concentrated. The residue was purified by column chromatography (eluted with dichloromethane:methanol=9:1) to give 4-(1H-1,2,3-benzotriazol-6-yl)-N-[3,5-bis(trifluoromethyl)phenyl]phthalazin-1-amine (70 mg, 0.147 mmol, 76% yield) as a colorless solid. ¹H NMR (400 MHz, d₆-DMSO): δ 9.97 (s, 1H), 8.83 (s, 2H), 8.72 (d, 1H), 8.16 (s, 1H), 8.05 (m, 2H), 7.95 (m, 2H), 7.70 (d, 1H), 7.65 (s, 1H). MS (EI) for C₂₂H₁₂F₆N₆: 475 (MH⁺).

### Example 9

### N-[3,5-Bis(trifluoromethyl)phenyl]-4-(1,3-dihydro-2H-pyrrolo[3,4-c]pyridin-2-yl)phthalazin-1-amine

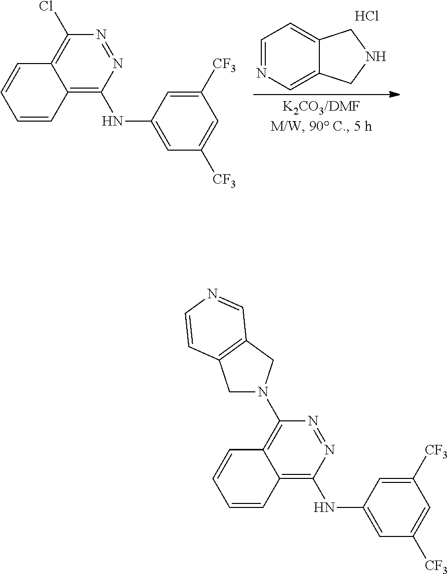

N-[3,5-Bis(trifluoromethyl)phenyl]-4-chlorophthalazin-1-amine (prepared according to the procedure for Intermediate 1) (80 mg, 0.204 mmol), 2,3-dihydro-1H-pyrrolo[3,4-c]pyridine hydrochloride salt (160 mg, 1.02 mmol) and potassium carbonate (141 mg, 1.02 mmol) were suspended in N,N-dimethylformamide (1.0 mL) and the mixture was irradiated with microwaves at 90° C. for 5 h. The mixture was purified by silica gel column chromatography (eluted with ethyl acetate:dichloromethane=1:1) to give the product. The product was washed with a minimal amount of dichloromethane and hexanes to afford N-[3,5-bis(trifluoromethyl)phenyl]-4-(1,3-dihydro-2H-pyrrolo[3,4-c]pyridin-2-yl)phthalazin-1-amine as a yellow solid (8 mg, 0.0168 mmol, 8% yield). ¹H NMR (600 MHz, d₆-DMSO): δ 9.56 (s, 1H), 8.67 (s, 1H), 8.65 (s, 2H), 8.54 (m, 3H), 8.08 (t, 1H), 8.02 (t, 1H), 7.57 (s, 1H), 7.50 (d, 1H), 5.25 (d, 4H). MS (EI) for C₂₃H₁₅F₆N₅: 476 (MH⁺).

### Example 10

### N-{3-[3-(Dimethylamino)propyl]-5-(trifluoromethyl)phenyl}-4-(3-methyl-1H-indazol-6-yl)phthalazin-1-amine trifluoroacetate salt

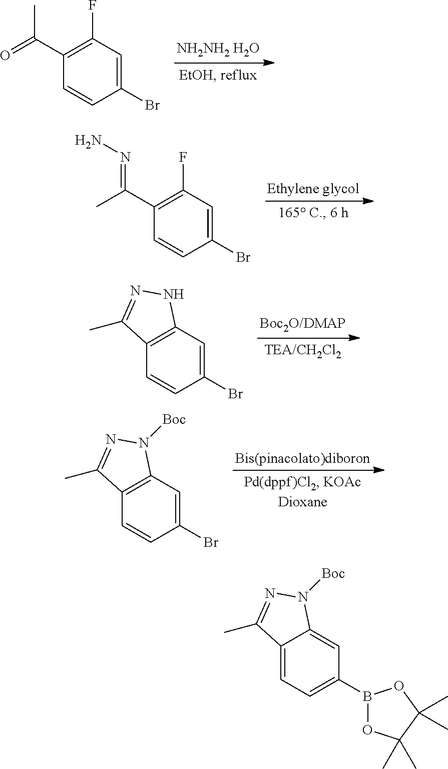

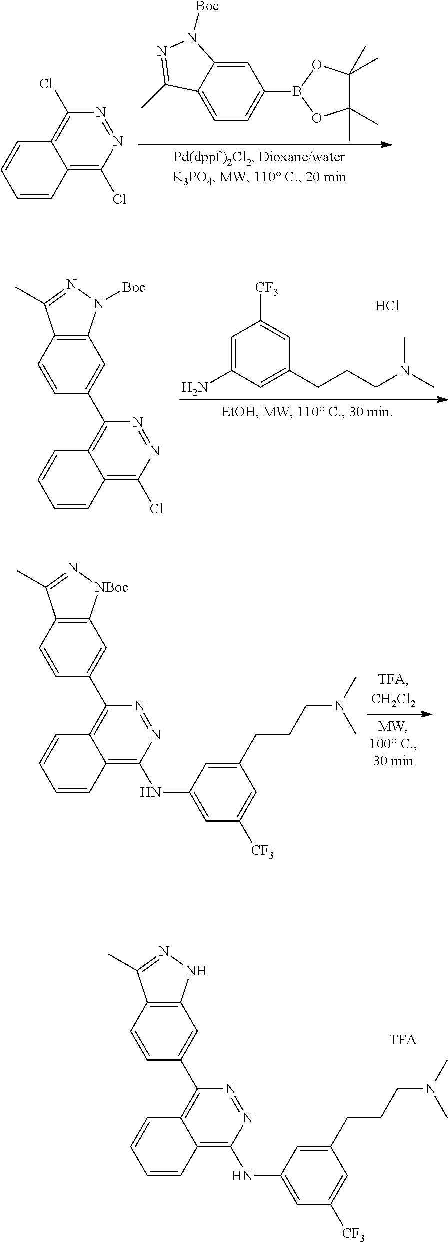

### 1-(4-Bromo-2-fluorophenyl)ethanone hydrazone

1-(4-Bromo-2-fluorophenyl)ethanone (prepared according to the procedure for Intermediate 4) (467 mg, 2.15 mmol) was dissolved in ethanol (5 mL) and was treated with hydrazine (0.168 mL, 2.79 mmol) at reflux for 8 h. The reaction mixture was then concentrated and was purified by silica gel chromatography (eluted with hexanes:ethyl acetate=4:1) to give 1-(4-bromo-2-fluorophenyl)ethanone hydrazone (420 mg, 84% yield) as a yellow colorless solid. ¹H NMR (400 MHz, CDCl₃): δ 7.39 (m, 1H), 7.23 (m, 2H), 5.41 (br s, 2H), 2.10 (d, 3H).

### 6-Bromo-3-methyl-1H-indazole

1-(4-Bromo-2-fluorophenyl)ethanone hydrazone (420 mg, 1.817 mmol) was dissolved in ethylene glycol (5 mL) and was heated at 165° C. for 6 h after which time the cooled reaction mixture was poured into water (15 mL). The aqueous mixture was neutralized using a small amount of saturated aqueous sodium bicarbonate to afford a pale yellow precipitate. The solid was filtered, was washed with water and was dried to afford 6-bromo-3-methyl-1H-indazole (330 mg, 86% yield) as a pale yellow solid. ¹H NMR (400 MHz, CDCl₃): δ 9.94 (br s, 1H), 7.60 (s, 1H), 7.53 (d, 1H), 7.23 (d, 1H), 2.62 (s, 3H).

### tert-Butyl 6-bromo-3-methyl-1H-indazole-1-carboxylate

To a solution of 6-bromo-3-methyl-1H-indazole (330 mg, 1.56 mmol) in dichloromethane (15 mL) was added triethylamine (0.67 mL, 4.68 mmol), di-tert-butyl dicarbonate (443 mg, 2.03 mmol) and 4-(dimethylamino)pyridine (19 mg, 0.156 mmol) at 0° C. The resulting solution was stirred for 3 h and the reaction mixture was washed with water, was dried over magnesium sulfate, was filtered and was concentrated. The residue was purified by column chromatography (eluted with ethyl acetate:hexanes=1:6) to give tert-butyl 6-bromo-3-methyl-1H-indazole-1-carboxylate (366 mg, 75% yield) as a light pink solid. ¹H NMR (400 MHz, CDCl₃): δ 8.31 (s, 1H), 7.46 (d, 1H), 7.37 (d, 1H), 2.54 (s, 3H), 1.60 (s, 9H).

### tert-Butyl 3-methyl-6-(4,4,5,5-tetramethyl-1,3,2-dioxaborolan-2-yl)-1H-indazole-1-carboxylate

tert-Butyl 6-bromo-3-methyl-1H-indazole-1-carboxylate (360 mg, 1.157 mmol), bis(pinacolato)diboron (150 mg, 1.73 mmol), Pd(dppf)₂Cl₂ dichloromethane adduct (94 mg, 0.1157 mmol) and potassium acetate (340 mg, 3.471 mmol) were suspended in 1,4-dioxane (15 mL). The mixture was stirred at 80° C. overnight and then was cooled to room temperature. The mixture was diluted with ethyl acetate, was filtered through a celite pad and the filtrate was concentrated. The residue was purified by column chromatography (eluted with ethyl acetate:hexanes=1:6) to give tert-butyl 3-methyl-6-(4,4,5,5-tetramethyl-1,3,2-dioxaborolan-2-yl)-1H-indazole-1-carboxylate (280 mg, 67% yield) as a colorless solid. ¹H NMR (400 MHz, CDCl₃): δ 8.62 (s, 1H), 7.71 (d, 1H), 7.64 (d, 1H), 2.60 (s, 3H), 1.73 (s, 9H), 1.36 (s, 12H).

### tert-Butyl 6-(4-chlorophthalazin-1-yl)-3-methyl-1H-indazole-1-carboxylate

1,4-Dichlorophthalazine (150 mg, 0.753 mmol), tert-butyl 3-methyl-6-(4,4,5,5-tetramethyl-1,3,2-dioxaborolan-2-yl)-1H-indazole-1-carboxylate (270 mg, 0.753 mmol), Pd(dppf)₂Cl₂ dichloromethane adduct (61 mg, 0.0753 mmol) and potassium phosphate tribasic (479 mg, 2.259 mmol) were suspended in 1,4-dioxane (10 mL) and water (1.0 mL) and the mixture was irradiated with microwaves at 110° C. for 20 minutes. The mixture was treated with 1N aqueous sodium hydroxide (10 mL) and was extracted with ethyl acetate. The organic layer was washed with 1N aqueous sodium hydroxide (2×10 mL), was dried over magnesium sulfate, was filtered and was concentrated. The residue was purified by column chromatography (eluted with ethyl acetate:hexanes=1:2) to give tert-butyl 6-(4-chlorophthalazin-1-yl)-3-methyl-1H-indazole-1-carboxylate (146 mg, 48% yield) as a colorless solid. ¹H NMR (400 MHz, d₆-DMSO): δ 8.43 (d, 1H), 8.37 (s, 1H), 8.22 (t, 1H), 8.17 (t, 1H), 8.09 (d, 2H), 7.71 (d, 1H), 2.62 (s, 3H), 1.59 (s, 9H).

### tert-Butyl 6-[4-({3-[3-(dimethylamino)propyl]-5-(trifluoromethyl)phenyl}amino)phthalazin-1-yl]-3-methyl-1H-indazole-1-carboxylate

tert-Butyl 6-(4-chlorophthalazin-1-yl)-3-methyl-1H-indazole-1-carboxylate (138 mg, 0.349 mmol) and 3-[3-(dimethylamino)propyl]-5-(trifluoromethyl)aniline hydrochloride salt (the hydrochloride salt of the aniline prepared according to the procedure for Intermediate 6) (148 mg, 0.524 mmol) were suspended in ethanol (4.0 mL) and the mixture was irradiated with microwaves at 100° C. for 30 minutes. The mixture was concentrated and was purified by column chromatography (eluted with dichloromethane:methanol=1:9 to 1:5) to give tert-butyl 6-[4-({3-[3-(dimethylamino)propyl]-5-(trifluoromethyl)phenyl}amino)phthalazin-1-yl]-3-methyl-1H-indazole-1-carboxylate (31 mg, 15% yield) as a colorless solid. ¹H NMR (400 MHz, CDCl₃): δ 8.47 (m, 2H), 8.14 (s, 1H), 7.96 (m, 3H), 7.88 (t, 1H), 7.74 (m, 2H), 7.62 (d, 1H), 7.03 (s, 1H), 2.71 (t, 2H), 2.64 (m, 5H), 2.43 (s, 6H), 1.96 (t, 2H), 1.60 (s, 9H).

### N-{3-[3-(Dimethylamino)propyl]-5-(trifluoromethyl)phenyl}-4-(3-methyl-1H-indazol-6-yl)phthalazin-1-amine trifluoroacetate salt

To a solution of tert-butyl 6-[4-({3-[3-(dimethylamino)propyl]-5-(trifluoromethyl)phenyl}amino)phthalazin-1-yl]-3-methyl-1H-indazole-1-carboxylate (31 mg, 0.0512 mmol) in dichloromethane (5 mL) was carefully added trifluoroacetic acid (0.5 mL). The mixture was irradiated with microwaves at 100° C. for 30 minutes. The mixture was concentrated and was washed with a minimal amount of diethyl ether to afford N-[3-[3-(dimethylamino)propyl]-5-(trifluoromethyl)phenyl]-4-(3-methyl-1H-indazol-6-yl)phthalazin-1-amine trifluoroacetate salt as a yellow solid (20 mg, 0.0396 mmol, 84% yield). ¹H NMR (400 MHz, d₆-DMSO): δ 9.61 (s, 1H), 8.79 (m, 1H), 8.16 (m, 2H), 8.02 (m, 2H), 7.99 (d, 1H), 7.76 (s, 1H), 7.38 (m, 2H), 7.36 (d, 1H), 3.03 (m, 2H), 2.70 (m, 8H), 2.61 (s, 3H), 2.02 (m, 2H). MS (EI) for C₂₈H₂₇F₃N₆: 505 (MH⁺).

### Example 11

### N-[3,5-Bis(trifluoromethyl)phenyl]-4-{3-[(methylamino)methyl]-1H-indazol-6-yl}phthalazin-1-amine

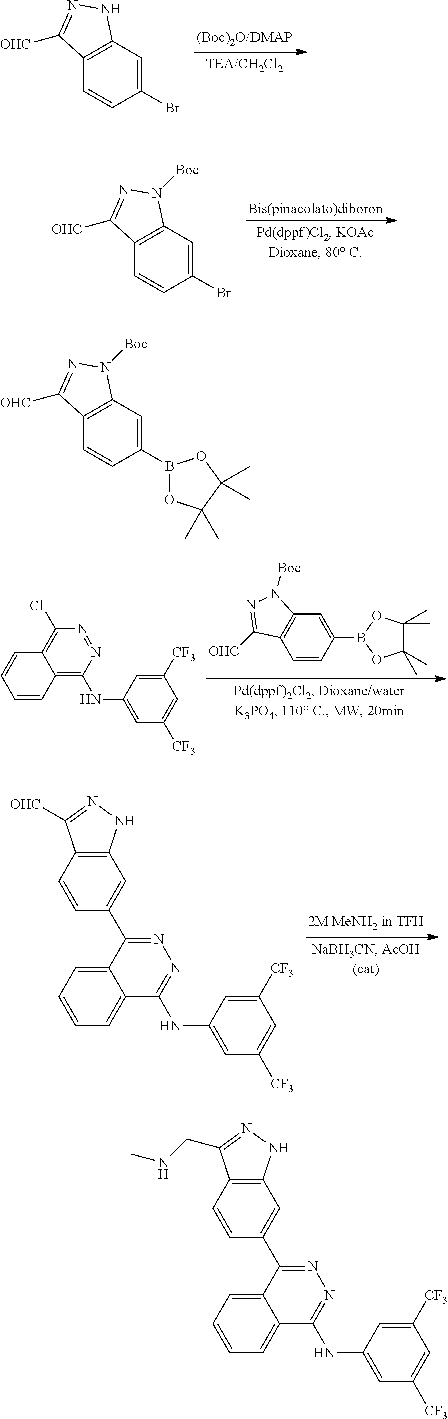

### tert-Butyl 6-bromo-3-formyl-1H-indazole-1-carboxylate

To a solution of the 6-bromo-1H-indazole-3-carbaldehyde (500 mg, 2.22 mmol) in dichloromethane (125 mL) was added triethylamine (0.34 mL, 2.44 mmol), di-tert-butyl dicarbonate (970 mg, 4.444 mmol) and 4-(dimethylamino)pyridine (27 mg, 0.222 mmol) at 0° C. The mixture was slowly warmed to room temperature and was washed with water and brine, was dried over magnesium sulfate, was filtered and was concentrated in vacuo. The crude material was purified by silica gel column chromatography (eluted with hexanes:ethyl acetate=3:1, R_{f}=0.7 in hexanes:ethyl acetate=2:1) to give tert-butyl 6-bromo-3-formyl-1H-indazole-1-carboxylate (746 mg, 2.294 mmol, 99% yield) as a colorless solid. ¹H NMR (400 MHz, CDCl₃): δ 10.30 (s, 1H), 8.42 (s, 1H), 8.18 (d, 1H), 7.57 (d, 1H), 1.76 (s, 9H), 1.52 (s, 12H).

### tert-Butyl 3-formyl-6-(4,4,5,5-tetramethyl-1,3,2-dioxaborolan-2-yl)-1H-indazole-1-carboxylate

tert-Butyl 6-bromo-3-formyl-1H-indazole-1-carboxylate (745 mg, 2.294 mmol), bis(pinacolato)diboron (874 mg, 3.441 mmol), Pd(dppf)₂Cl₂ dichloromethane adduct (187 mg, 0.229 mmol) and potassium acetate (675 mg, 6.874 mmol) were suspended in 1,4-dioxane (50 mL). The mixture was stirred at 80° C. overnight and then was cooled to room temperature. The mixture was diluted with ethyl acetate, was filtered through a celite pad and the filtrate was concentrated. The residue was purified by column chromatography (hexanes:ethyl acetate=3:1, R_{f}=0.4 in hexanes:ethyl acetate=3:1) to give tert-butyl 3-formyl-6-(4,4,5,5-tetramethyl-1,3,2-dioxaborolan-2-yl)-1H-indazole-1-carboxylate (687 mg, 1.846 mmol, 80% yield) as a colorless foam. ¹H NMR (400 MHz, CDCl₃): δ 10.37 (s, 1H), 8.67 (s, 1H), 8.29 (d, 1H), 7.84 (d, 1H), 1.77 (s, 9H), 1.37 (s, 12H).

### 6-(4-{[3,5-Bis(trifluoromethyl)phenyl]amino}phthalazin-1-yl)-1H-indazole-3-carbaldehyde

N-[3,5-Bis(trifluoromethyl)phenyl]-4-chlorophthalazin-1-amine (prepared according to the procedure for Intermediate 1) (501 mg, 1.279 mmol), tert-butyl 3-formyl-6-(4,4,5,5-tetramethyl-1,3,2-dioxaborolan-2-yl)-1H-indazole-1-carboxylate (476 mg, 1.279 mmol), Pd(dppf)₂Cl₂ dichloromethane adduct (104 mg, 0.128 mmol) and potassium phosphate tribasic (814 mg, 3.836 mmol) were suspended in 1,4-dioxane (4 mL) and water (0.4 mL) and the mixture was irradiated with microwaves at 110° C. for 20 minutes. The mixture was treated with 1N aqueous sodium hydroxide (10 mL) and was extracted with ethyl acetate. The organic layer was washed with 1N aqueous sodium hydroxide (2×10 mL), was dried over magnesium sulfate, was filtered and was concentrated. The residue was purified by column chromatography (eluted with hexanes:ethyl acetate=3:1) to give 6-(4-{[3,5-bis(trifluoromethyl)phenyl]amino}phthalazin-1-yl)-1H-indazole-3-carbaldehyde (108 mg, 0.215 mmol, 17% yield) as a colorless foam. ¹H NMR (400 MHz, CDCl₃): δ 10.21 (s, 1H), 8.46 (s, 1H), 8.38-8.30 (m, 2H), 8.01 (d, 1H), 7.98-7.80 (m, 4H), 7.53-7.40 (m, 2H).

### N-[3,5-Bis(trifluoromethyl)phenyl]-4-{3-[(methylamino)methyl]-1H-indazol-6-yl}phthalazin-1-amine

To a solution of 6-(4-{[3,5-bis(trifluoromethyl)phenyl]amino}phthalazin-1-yl)-1H-indazole-3-carbaldehyde (70 mg, 0.140 mmol) in methanol (1 mL) was added methylamine (2M in tetrahydrofuran; 2 mL), sodium cyanoborohydride (10 mg, 0.150 mmol) and glacial acetic acid (2 drops). The vessel was sealed and heated to 45° C. for 2 days. Upon completion of the reaction, the solvent was removed. The crude mixture was purified by column chromatography (eluted with dichloromethane:methanol=4:1) to give N-[3,5-bis(trifluoromethyl)phenyl]-4-{3-[(methylamino)methyl]-1H-indazol-6-yl}phthalazin-1-amine (20 mg, 0.039 mmol, 39% yield) as a colorless foam. ¹H NMR (400 MHz, d₆-DMSO): δ 13.57 (s, 1H), 10.08 (s, 1H), 8.99 (s, 2H), 8.86 (d, 1H), 8.27-8.22 (m, 2H), 8.16-8.08 (m, 2H), 7.98 (s, 1H), 7.84 (s, 1H), 7.61 (d, 1H), 4.59 (s, 2H), 2.74 (s, 3H). MS (EI) for C₂₅H₁₈F₆N₆: 517 (MH⁺).

### Example 12

### 4-[4-(4-{[3,5-Bis(trifluoromethyl)phenyl]amino}phthalazin-1-yl)phenyl]-2,4-dihydro-3H-1,2,4-triazol-3-one

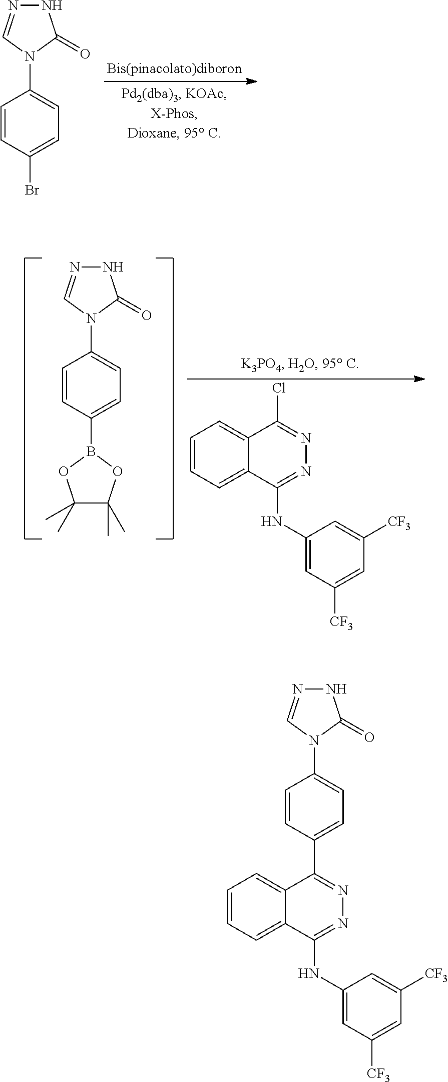

4-(4-Bromophenyl)-2,4-dihydro-3H-1,2,4-triazol-3-one (130 mg, 0.54 mmol), bis(pinacolato)diboron (150 mg, 0.59 mmol), Pd₂(dba)₃ (50 mg, 0.05 mmol), X-Phos (55 mg, 0.12 mmol) and potassium acetate (110 mg, 1.12 mmol) were suspended in 1,4-dioxane (4 mL). The mixture was stirred at 95° C. for 3 h and then N-[3,5-bis(trifluoromethyl)phenyl]-4-chlorophthalazin-1-amine (prepared according to the procedure for Intermediate 1) (100 mg, 0.25 mmol), potassium phosphate tribasic (345 mg, 1.63 mmol) and water (0.5 mL) were added and the mixture was flushed with argon and was heated at 95° C. for 5 h. The mixture was purified by preparative HPLC and then by column chromatography (eluted with ethyl acetate:hexanes) to give 4-[4-(4-{[3,5-bis(trifluoromethyl)phenyl]amino}phthalazin-1-yl)phenyl]-2,4-dihydro-3H-1,2,4-triazol-3-one (22 mg, 0.043 mmol, 17% yield) as a colorless solid. ¹H NMR (400 MHz, d₆-DMSO) δ 12.08 (br s, 1H), 9.93 (br s, 1H), 8.83 (s, 2H), 8.70 (d, 1H), 8.53 (s, 1H), 8.15-8.09 (m, 1H), 8.05-7.98 (m, 1H), 7.98-7.92 (m, 3H), 7.85-7.80 (m, 2H), 7.69 (s, 1H). MS (EI) for C₂₄H₁₄F₆N₆O: 517 (MH⁺).

### Example 13

### Methyl 4-(4-{[3-(trifluoromethyl)phenyl]amino}pyrido[3,4-d]pyridazin-1-yl)benzoate

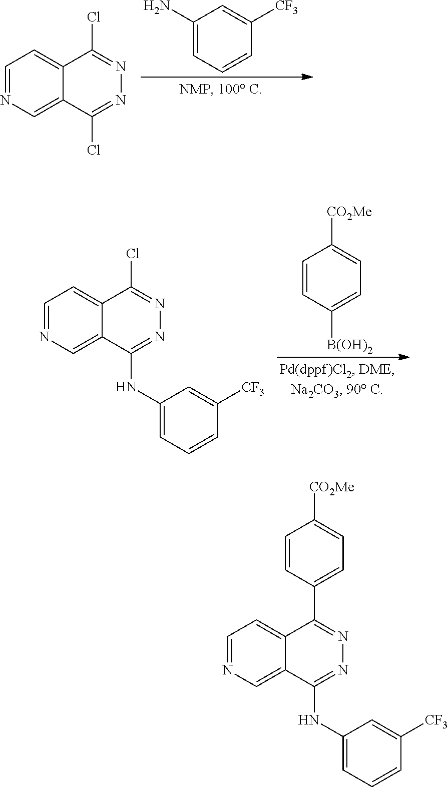

### 1-Chloro-N-(3-(trifluoromethyl)phenyl)pyrido[4,3-d]pyridazin-4-amine

A pressure vessel was charged with 1,4-dichloropyrido[4,3-d]pyridazine (prepared according to the procedures in WO2009/035568A1) (66 mg, 0.33 mmol, 1.0 eq.), 3-trifluoromethyl aniline (0.05 mL, 0.39 mmol, 1.2 eq.) and 1-methyl-2-pyrrolidinone (0.1 mL). The reaction was sealed and was heated to 105° C. for 1 h. Analysis showed mono-addition as the major product with a minor amount of bis-addition product. Regioisomeric mono-addition products were not observed. The reaction was then cooled to room temperature and was diluted with water and saturated sodium bicarbonate solution. The mixture was extracted with ethyl acetate (2×). The combined organic layer was dried with sodium sulfate, was filtered and was concentrated to give the crude product. The product was purified by column chromatography (eluted with hexanes:ethyl acetate=4:1 to 1:1) to afford 1-chloro-N-(3-(trifluoromethyl)phenyl)pyrido[4,3-d]pyridazin-4-amine (41 mg, 38% yield). MS (EI) for C₁₄H₈ClF₃N₄: 325 (MH⁺), Chlorine isotope pattern.

### Methyl 4-(4-{[3-(trifluoromethyl)phenyl]amino}pyrido[3,4-d]pyridazin-1-yl)benzoate

A pressure vessel was charged with 1-chloro-N-(3-(trifluoromethyl)phenyl)pyrido[4,3-d]pyridazin-4-amine (41 mg, 0.13 mmol, 1.0 eq.), 4-(methoxycarbonyl)phenylboronic acid (28 mg, 0.15 mmol, 1.2 eq.), 2.0M aqueous sodium carbonate (0.13 mL, 0.25 mmol, 2.0 eq.), and 1,2-dimethoxyethane (3 mL). The reaction was purged with nitrogen before adding Pd(dppf)₂Cl₂ (10 mg, 0.012 mmol, 0.1 eq.) and the mixture was sealed and was heated at 90° C. for 6 h. The reaction was then cooled to room temperature, was filtered through silica gel eluting with ethyl acetate and was concentrated to give the crude product. The product was purified by column chromatography (eluted with hexanes:ethyl acetate=1:1) to afford methyl 4-(4-{[3-(trifluoromethyl)phenyl]amino}pyrido[3,4-d]pyridazin-1-yl)benzoate (21 mg, 31% yield). ¹H NMR (400 MHz, d₆-DMSO) δ 10.04 (s, 1H), 9.27 (s, 0.5H), 9.15 (d, 0.5H), 9.02 (d, 0.5H), 8.56 (dd, 0.5H), 8.49 (d, 1H), 8.25 (t, 1H), 8.16 (dd, 2H), 7.90 (dd, 2H), 7.76 (d, 1H), 7.64 (t, 1H), 7.43 (d, 1H), 3.88 (s, 3H). MS (EI) for C₂₂H₁₅F₃N₄O₂: 425 (MH⁺).

The following compounds were synthesized in an analogous fashion to the compound described above.

### Methyl 4-(4-{[3-(1-methylethyl)phenyl]amino}pyrido[3,4-d]pyridazin-1-yl)benzoate

MS (EI) for C₂₄H₂₂N₄O₂: 399 (MH⁺).

### Example 14

### Methyl 4-(5-{[3-(trifluoromethyl)phenyl]amino}pyrido[2,3-d]pyridazin-8-yl)benzoate

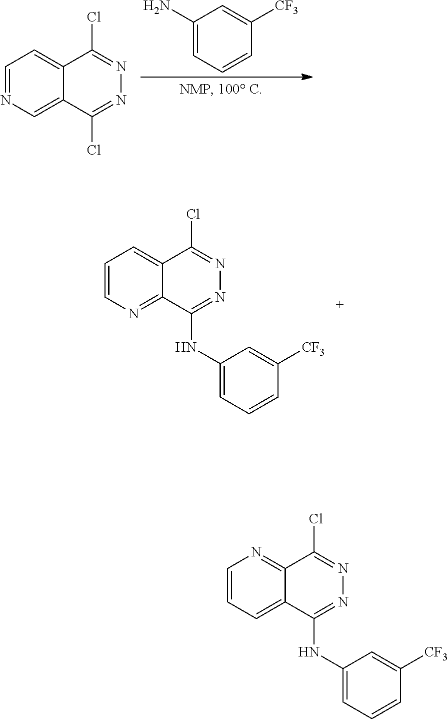

5-Chloro-N-(3-(trifluoromethyl)phenyl)pyrido[3,2-d]pyridazin-8-amine and 8-chloro-N-(3-(trifluoromethyl)phenyl)pyrido[2,3-d]pyridazin-5-amine: A pressure vessel was charged with 5,8-dichloropyrido[3,2-d]pyridazine (prepared according to the procedures in WO2009/035568A1) (100 mg, 0.51 mmol, 1.0 eq.), 3-trifluoromethyl aniline (0.64 mL, 0.45 mmol, 0.9 eq.) and 1-methyl-2-pyrrolidinone (0.5 mL). The reaction was sealed and heated to 100° C. for 23 minutes. Analysis showed a 1:1 mixture of regioisomeric mono-addition products. The reaction was cooled to room temperature, was diluted with saturated sodium bicarbonate solution. The mixture was then extracted with ethyl acetate (2×). The combined organic layer was dried over sodium sulfate, was filtered and was concentrated to give the crude product. The product was purified by column chromatography (eluted with hexanes:ethyl acetate=7:3 to 1:1) to afford 5-chloro-N-(3-(trifluoromethyl)phenyl)pyrido[3,2-d]pyridazin-8-amine and 8-chloro-N-(3-(trifluoromethyl)phenyl)pyrido[2,3-d]pyridazin-5-amine (54 mg and 52 mg respectively, 66% yield). MS (EI) for C₁₄H₈ClF₃N₄: 325 (MH⁺), Chlorine isotope pattern.

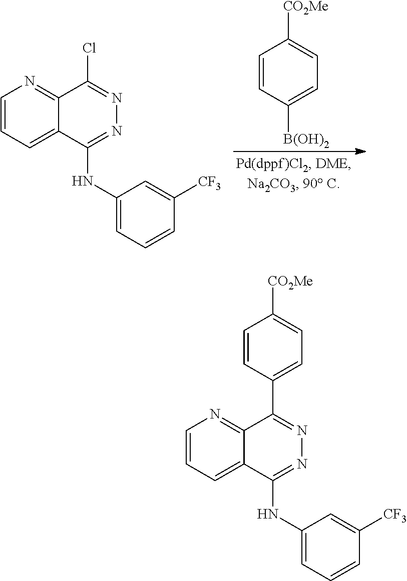 [SMILES: FC(F)(F)c1cccc(Nc2nnc(Cl)c3ncccc23)c1 ; COC(=O)c1ccc(B(O)O)cc1 ; COC(=O)c1ccc(-c2nnc(Nc3cccc(C(F)(F)F)c3)c3cccnc23)cc1]

### Methyl 4-(5-{[3-(trifluoromethyl)phenyl]amino}pyrido[2,3-d]pyridazin-8-yl)benzoate

A pressure vessel was charged with 8-chloro-N-(3-(trifluoromethyl)phenyl)pyrido[2,3-d]pyridazin-5-amine (52 mg, 0.16 mmol, 1.0 eq.), 4-(methoxycarbonyl)phenylboronic acid (37 mg, 0.21 mmol, 1.3 eq.), 2.0 M aqueous sodium carbonate (0.16 mL, 0.42 mmol, 2.0 eq.) and 1,2-dimethoxyethane (3 mL). The reaction was purged with nitrogen before adding Pd(dppf)₂Cl₂ (14 mg, 0.012 mmol, 0.1 eq.) and was sealed and was heated at 90° C. for 16 h. The reaction was cooled to room temperature, was filtered through silica gel eluting with ethyl acetate and was concentrated to give the crude product. The product was purified by column chromatography (eluted with hexanes:ethyl acetate=1:1) to afford methyl 4-(5-{[3-(trifluoromethyl)phenyl]amino}pyrido[2,3-d]pyridazin-8-yl)benzoate (25 mg, 35% yield). ¹H NMR (400 MHz, d₆-DMSO) δ 9.82 (br s, 1H), 9.33-9.21 (m, 1H), 9.08 (d, 1H), 8.46 (s, 1H), 8.23 (d, 1H), 8.15 (d, 2H), 8.12-8.01 (m, 3H), 7.62 (t, 1H), 7.40 (d, 1H), 3.87 (s, 3H). MS (EI) for C₂₂H₁₅F₃N₄O₂: 425 (MH⁺).

The following compounds were synthesized in an analogous fashion to the compound described above.

### Methyl 4-(8-{[3-(trifluoromethyl)phenyl]amino}pyrido[2,3-d]pyridazin-5-yl)benzoate

MS (EI) for C₂₂H₁₅F₃N₄O₂: 425 (MH⁺).

### Example 15

### Methyl 4-(4-{[3-(trifluoromethyl)phenyl]amino}isoquinolin-1-yl)benzoate

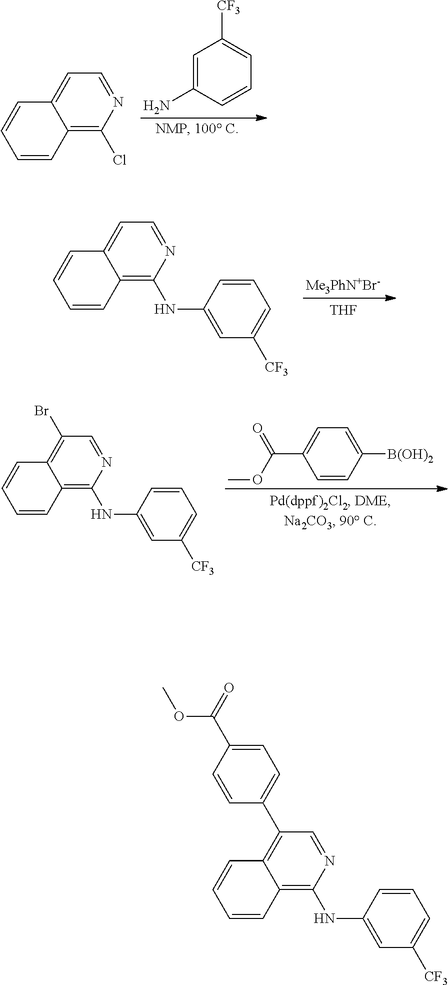

### N-(3-(Trifluoromethyl)phenyl)isoquinolin-1-amine

A pressure vessel was charged with 1-chloro-isoquinoline (1.5 g, 9.2 mmol, 1.0 eq.), 3-trifluoromethyl aniline (1.2 mL, 10.1 mmol, 1.1 eq.) and 1-methyl-2-pyrrolidinone (9 mL). The reaction was sealed and heated to 100° C. overnight. The reaction was then cooled to room temperature and was diluted with water. The mixture was extracted with ethyl acetate (2×). The combined organic portion was washed with brine, was dried over sodium sulfate, was filtered and was concentrated to give the crude product. The product was purified by column chromatography (eluted with hexanes:ethyl acetate=5:1 to 3:1) to afford N-(3-(trifluoromethyl)phenyl)isoquinolin-1-amine (2.0 g, 74% yield). MS (EI) for C₁₆H₁₁F₃N₂: 289 (MH⁺).

### 4-Bromo-N-(3-(trifluoromethyl)phenyl)isoquinolin-1-amine

A round bottom flask was charged with N-(3-(trifluoromethyl)phenyl)isoquinolin-1-amine (1.0 g, 3.5 mmol, 1.0 eq.), and tetrahydrofuran (6 mL). The mixture was cooled to 0° C. before adding dropwise trimethylphenylammonium tribromide (1.2 g, 3.15 mmol, 0.9 eq.) dissolved in 12 mL of tetrahydrofuran. The reaction was allowed to warm to room temperature slowly and was stirred overnight. The reaction was then poured into hexanes where an orange precipitate formed. The solid was collected by filtration and was dissolved in dichloromethane. The organic solution was washed with saturated sodium bicarbonate solution, water, was dried over sodium sulfate, was filtered and was concentrated to give the crude product. The product was purified by column chromatography (eluted with hexanes:ethyl acetate=95:5 to 90:10) to afford 4-bromo-N-(3-(trifluoromethyl)phenyl)isoquinolin-1-amine (0.93 g, 72% yield). MS (EI) for C₁₆H₁₀BrF₃N₂: 367 (MH⁺), Bromine isotope pattern.

### Methyl 4-(4-{[3-(trifluoromethyl)phenyl]amino}isoquinolin-1-yl)benzoate

A pressure vessel was charged with 4-bromo-N-(3-(trifluoromethyl)phenyl)isoquinolin-1-amine (140 mg, 0.38 mmol, 1.0 eq.), 4-(methoxycarbonyl)phenylboronic acid (75 mg, 0.42 mmol, 1.1 eq.), 2.0 M aqueous sodium carbonate (0.381 mL, 0.76 mmol, 2.0 eq.), and 1,2-dimethoxyethane (1 mL). The reaction was purged with nitrogen before adding Pd(dppf)₂Cl₂ (16 mg, 0.019 mmol, 0.05 eq.) and was sealed and heated to 100° C. for 16 h. The reaction was then cooled to room temperature and was absorbed onto silica gel. The product was purified by column chromatography (eluted with hexanes:ethyl acetate=80:20 to 40:60) to afford crude methyl 4-(4-{[3-(trifluoromethyl)phenyl]amino}isoquinolin-1-yl)benzoate. The compound was further purified by preparative HPLC to afford pure methyl 4-(4-{[3-(trifluoromethyl)phenyl]amino}isoquinolin-1-yl)benzoate (25 mg, 16% yield). ¹H NMR (400 MHz, d₆-DMSO) δ 9.65 (s, 1H), 8.64 (d, 1H), 8.34 (s, 1H), 8.25 (d, 1H), 8.09 (d, 1H), 8.04 (d, 1H), 7.85-7.70 (m, 3H), 7.66 (d, 2H), 7.56 (t, 1H), 7.29 (dd, 1H), 3.88 (s, 3H). MS (EI) for C₂₄H₁₇F₃N₂O₂: 423 (MH⁺).

The following compounds were synthesized in an analogous fashion to the compound described above.

### Methyl 4-(4-{[3-(1-methylethyl)phenyl]amino}isoquinolin-1-yl)benzoate

MS (EI) for C₂₆H₂₄N₂O₂: 397 (MH⁺).

### Example 16

### 4-(4-{[3,5-Bis(trifluoromethyl)phenyl]amino}phthalazin-1-yl)-N-methylbenzamide

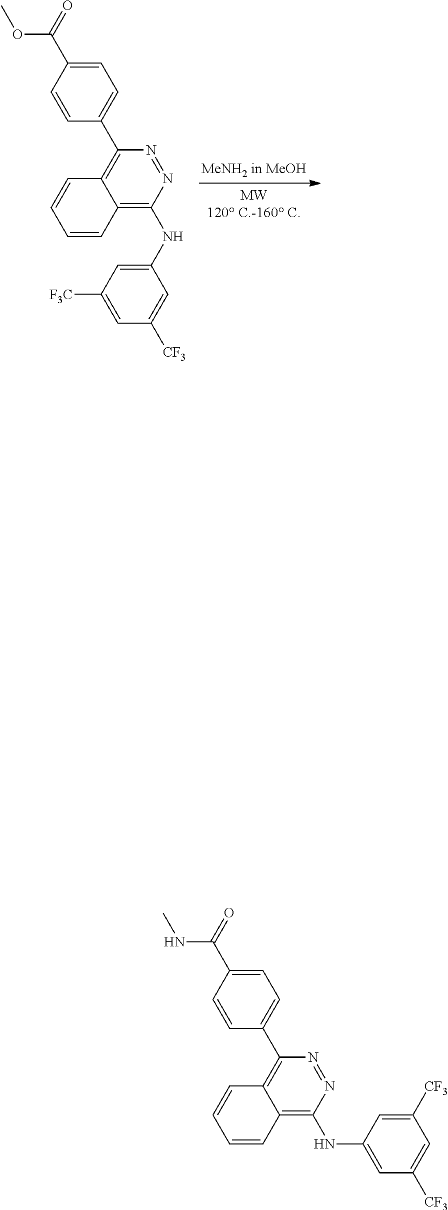

Methyl 4-(4-{[3,5-bis(trifluoromethyl)phenyl]amino}phthalazin-1-yl)benzoate (prepared according to the procedure for Example 1) (32 mg, 0.08 mmol) was irradiated with 2M methylamine in methanol (1 mL) in the microwave at 120° C. for 20 minutes, then 150° C. for 20 minutes and then at 160° C. for 1 h. The mixture was purified by preparative HPLC to afford 4-(4-{[3,5-bis(trifluoromethyl)phenyl]amino}phthalazin-1-yl)-N-methylbenzamide (3 mg, 0.006 mmol, 8% yield). ¹H NMR (400 MHz, d₆-DMSO) δ 9.98 (s, 1H), 8.85 (s, 2H), 8.73 (d, 1H), 8.63 (q, 1H), 8.14 (t, 1H), 8.07-8.01 (m, 3H), 7.95 (d, 1H), 7.80 (d, 2H), 7.72 (s, 1H), 2.85 (d, 3H). MS (EI) for C₂₄H₁₆F₆N₄O: 491 (MH⁺).

### Biological Examples

### JAK1 Kinase Assay Used to Test Compounds

JAK1 Kinase activity was measured as the percent of ATP consumed following the kinase reaction using luciferase-luciferin-coupled chemiluminescence. The reaction was conducted in white medium binding 384-well microtiter plates. The kinase reaction were initiated by combining test compounds, 8 nM of recombinant JAK1 enzyme (Insect expressed: V866-K1155), 30 μM of IRS-1 peptide (Y608) and 2 μM ATP in buffer containing 20 mM Hepes (pH 7.5), 10 mM MgCl2, 0.01% Brij, 5% glycerol and 1 mM DTT in a 20 μL volume. The reaction mixture was incubated at ambient temperature for 2 hours. Following the kinase reaction, a 20 μL aliquot of KinaseGlo was added to stop the reaction and measure remaining ATP via detection of chemiluminescence using the Envison reader. Total ATP consumption was limited to 25-60%. Table 2 displays JAK1 IRS-1tide IC₅₀, coded as follows: A≦150 nM; 150 nM<B≦500 nM; 500 nM<C≦1000 nM; 1000 nM<D≦2000 nM; and 2000 nM<E≦30,000 nM.

TABLE 2

| COMPOUND | | JAK1 IRS-1tide |
|---|---|---|
| NO. | STRUCTURE | Chemilum (IC50) (nM) |
| 1 | 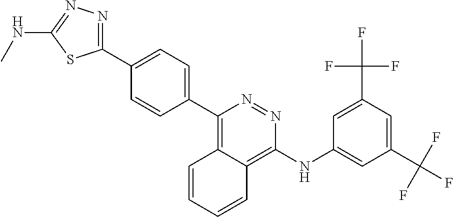 [SMILES: CNc1nnc(-c2ccc(-c3nnc(Nc4cc(C(F)(F)F)cc(C(F)(F)F)c4)c4ccccc34)cc2)s1] | E |
| 4 | 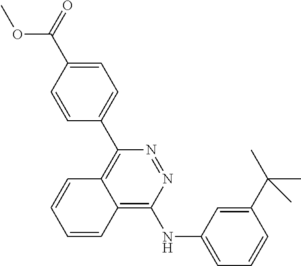 [SMILES: COC(=O)c1ccc(-c2nnc(Nc3cccc(C(C)(C)C)c3)c3ccccc23)cc1] | C |
| 5 | 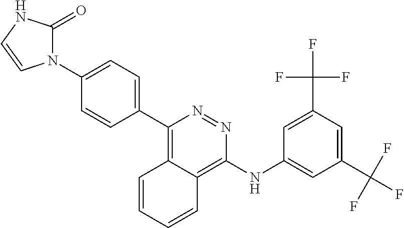 [SMILES: O=c1[nH]ccn1-c1ccc(-c2nnc(Nc3cc(C(F)(F)F)cc(C(F)(F)F)c3)c3ccccc23)cc1] | A |
| 8 | 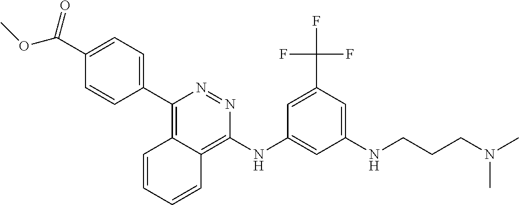 | B |
| 9 | 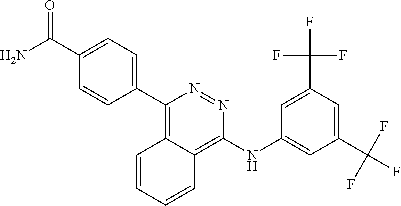 [SMILES: NC(=O)c1ccc(-c2nnc(Nc3cc(C(F)(F)F)cc(C(F)(F)F)c3)c3ccccc23)cc1] | D |
| 10 | 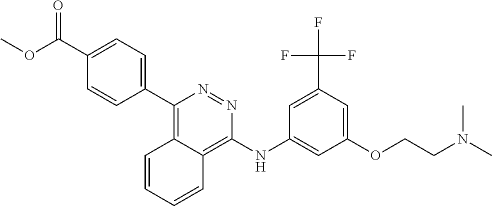 | B |
| 12 | 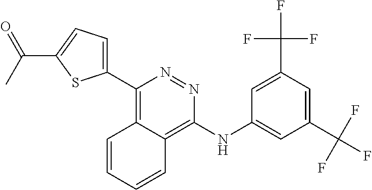 [SMILES: CC(=O)c1ccc(-c2nnc(Nc3cc(C(F)(F)F)cc(C(F)(F)F)c3)c3ccccc23)s1] | E |
| 13 | 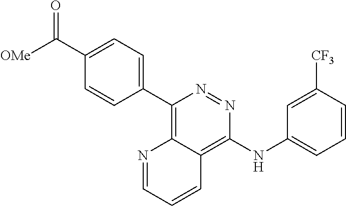 [SMILES: COC(=O)c1ccc(-c2nnc(Nc3cccc(C(F)(F)F)c3)c3cccnc23)cc1] | C |
| 14 | 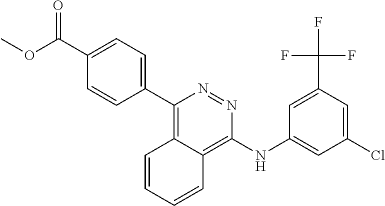 [SMILES: COC(=O)c1ccc(-c2nnc(Nc3cc(Cl)cc(C(F)(F)F)c3)c3ccccc23)cc1] | A |
| 15 | 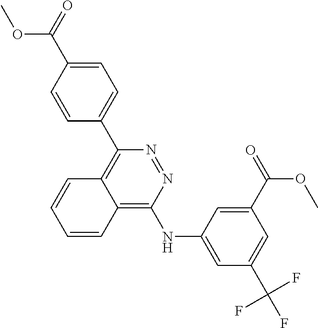 | E |
| 16 | 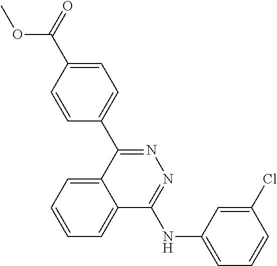 [SMILES: COC(=O)c1ccc(-c2nnc(Nc3cccc(Cl)c3)c3ccccc23)cc1] | B |
| 17 | 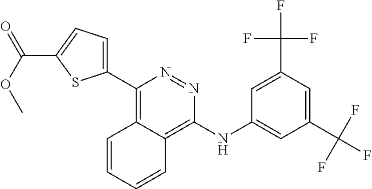 [SMILES: COC(=O)c1ccc(-c2nnc(Nc3cc(C(F)(F)F)cc(C(F)(F)F)c3)c3ccccc23)s1] | E |
| 18 | 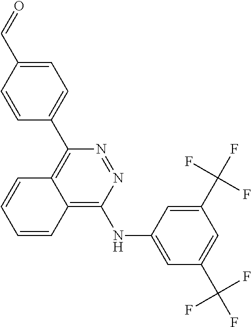 [SMILES: O=Cc1ccc(-c2nnc(Nc3cc(C(F)(F)F)cc(C(F)(F)F)c3)c3ccccc23)cc1] | E |
| 19 | 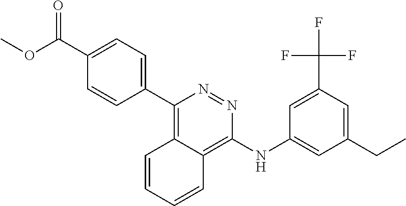 [SMILES: CCc1cc(Nc2nnc(-c3ccc(C(=O)OC)cc3)c3ccccc23)cc(C(F)(F)F)c1] | A |
| 20 | 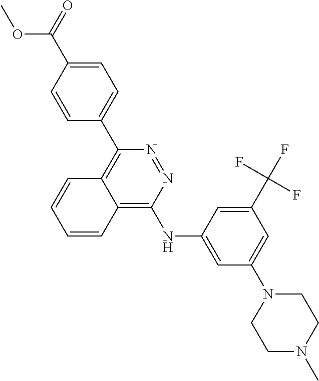 | D |
| 21 | 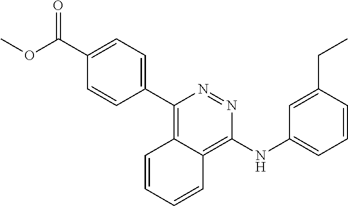 [SMILES: CCc1cccc(Nc2nnc(-c3ccc(C(=O)OC)cc3)c3ccccc23)c1] | B |
| 22 | 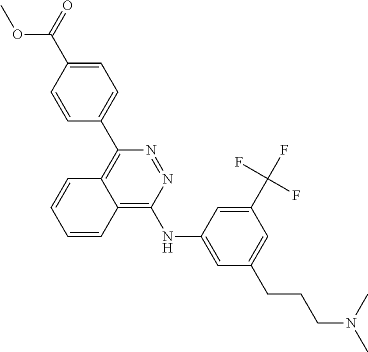 | A |
| 23 | 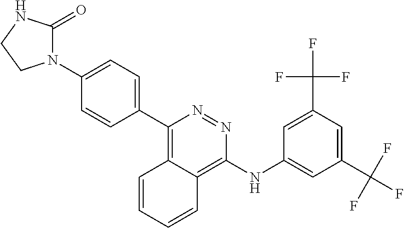 [SMILES: O=C1NCCN1c1ccc(-c2nnc(Nc3cc(C(F)(F)F)cc(C(F)(F)F)c3)c3ccccc23)cc1] | A |
| 24 | 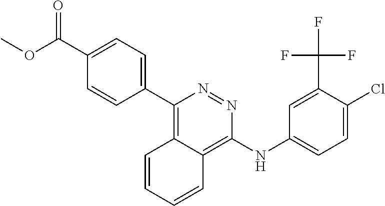 [SMILES: COC(=O)c1ccc(-c2nnc(Nc3ccc(Cl)c(C(F)(F)F)c3)c3ccccc23)cc1] | C |
| 26 | 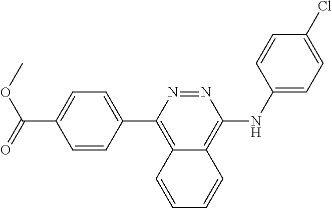 [SMILES: COC(=O)c1ccc(-c2nnc(Nc3ccc(Cl)cc3)c3ccccc23)cc1] | D |
| 27 | 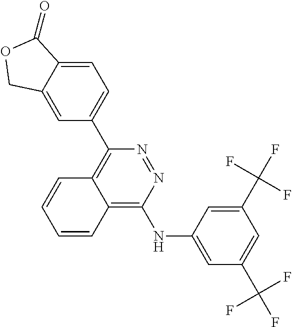 [SMILES: O=C1OCc2cc(-c3nnc(Nc4cc(C(F)(F)F)cc(C(F)(F)F)c4)c4ccccc34)ccc21] | E |
| 28 | 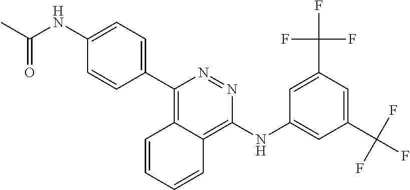 [SMILES: CC(=O)Nc1ccc(-c2nnc(Nc3cc(C(F)(F)F)cc(C(F)(F)F)c3)c3ccccc23)cc1] | C |
| 29 | 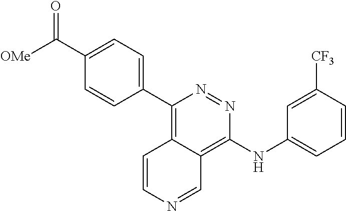 [SMILES: COC(=O)c1ccc(-c2nnc(Nc3cccc(C(F)(F)F)c3)c3cnccc23)cc1] | B |
| 30 | 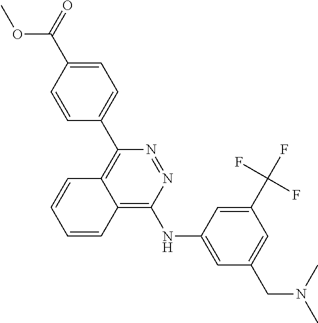 | C |
| 32 | 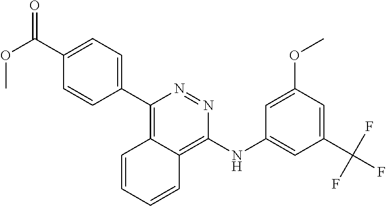 [SMILES: COC(=O)c1ccc(-c2nnc(Nc3cc(OC)cc(C(F)(F)F)c3)c3ccccc23)cc1] | C |
| 33 | 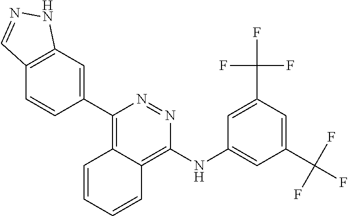 [SMILES: FC(F)(F)c1cc(Nc2nnc(-c3ccc4cn[nH]c4c3)c3ccccc23)cc(C(F)(F)F)c1] | A |
| 34 | 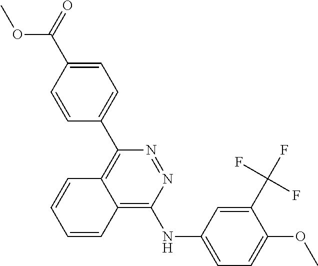 | C |
| 35 | 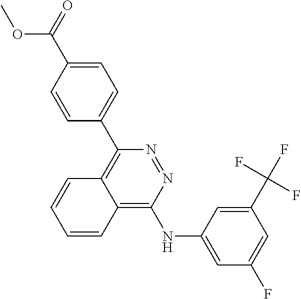 [SMILES: COC(=O)c1ccc(-c2nnc(Nc3cc(F)cc(C(F)(F)F)c3)c3ccccc23)cc1] | B |
| 36 | 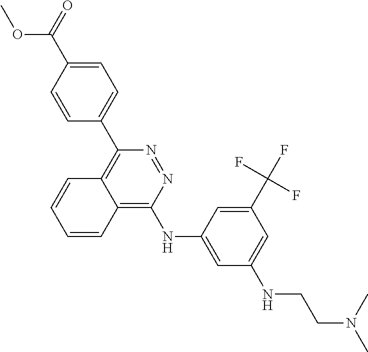 | B |
| 38 | 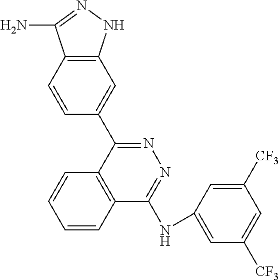 [SMILES: Nc1n[nH]c2cc(-c3nnc(Nc4cc(C(F)(F)F)cc(C(F)(F)F)c4)c4ccccc34)ccc12] | A |
| 39 | 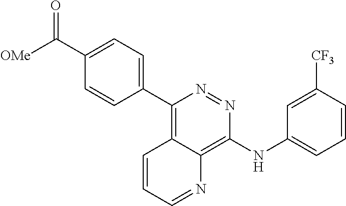 [SMILES: COC(=O)c1ccc(-c2nnc(Nc3cccc(C(F)(F)F)c3)c3ncccc23)cc1] | E |
| 40 | 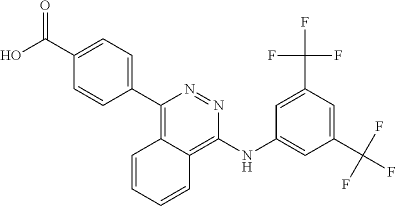 [SMILES: O=C(O)c1ccc(-c2nnc(Nc3cc(C(F)(F)F)cc(C(F)(F)F)c3)c3ccccc23)cc1] | E |
| 43 | 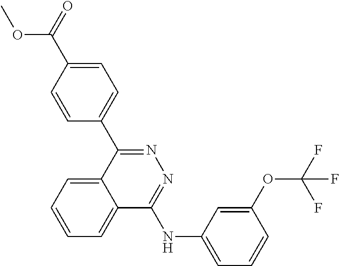 [SMILES: COC(=O)c1ccc(-c2nnc(Nc3cccc(OC(F)(F)F)c3)c3ccccc23)cc1] | B |
| 44 | 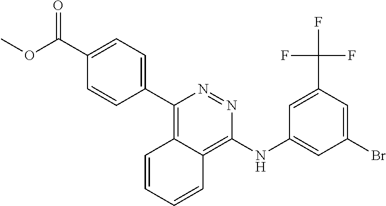 [SMILES: COC(=O)c1ccc(-c2nnc(Nc3cc(Br)cc(C(F)(F)F)c3)c3ccccc23)cc1] | B |
| 45 | 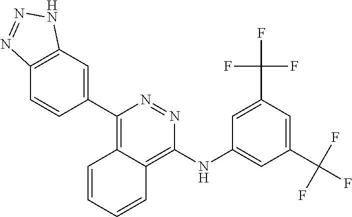 [SMILES: FC(F)(F)c1cc(Nc2nnc(-c3ccc4nn[nH]c4c3)c3ccccc23)cc(C(F)(F)F)c1] | D |
| 47 | 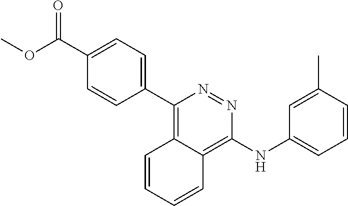 [SMILES: COC(=O)c1ccc(-c2nnc(Nc3cccc(C)c3)c3ccccc23)cc1] | C |
| 48 | 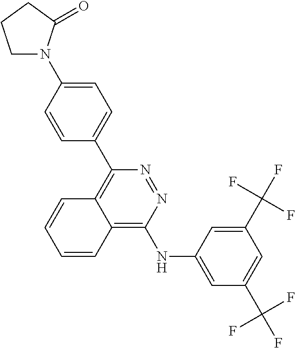 [SMILES: O=C1CCCN1c1ccc(-c2nnc(Nc3cc(C(F)(F)F)cc(C(F)(F)F)c3)c3ccccc23)cc1] | E |
| 49 | 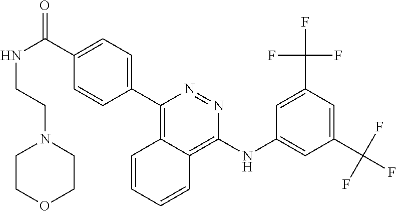 [SMILES: O=C(NCCN1CCOCC1)c1ccc(-c2nnc(Nc3cc(C(F)(F)F)cc(C(F)(F)F)c3)c3ccccc23)cc1] | C |
| 50 | 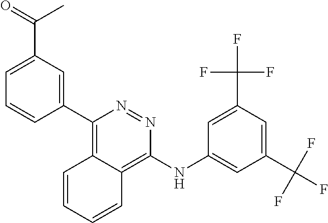 [SMILES: CC(=O)c1cccc(-c2nnc(Nc3cc(C(F)(F)F)cc(C(F)(F)F)c3)c3ccccc23)c1] | E |
| 51 | 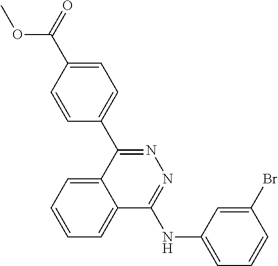 [SMILES: COC(=O)c1ccc(-c2nnc(Nc3cccc(Br)c3)c3ccccc23)cc1] | B |
| 52 | 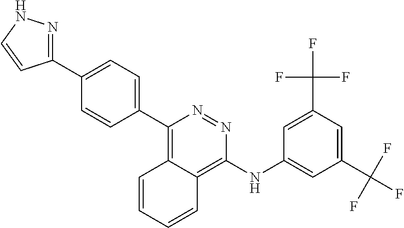 [SMILES: FC(F)(F)c1cc(Nc2nnc(-c3ccc(-c4cc[nH]n4)cc3)c3ccccc23)cc(C(F)(F)F)c1] | B |
| 53 | 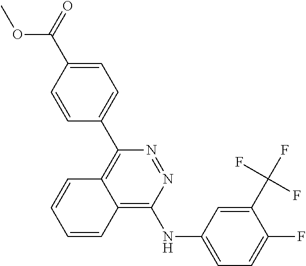 [SMILES: COC(=O)c1ccc(-c2nnc(Nc3ccc(F)c(C(F)(F)F)c3)c3ccccc23)cc1] | C |
| 54 | 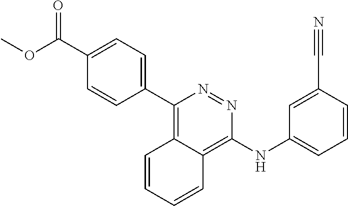 [SMILES: COC(=O)c1ccc(-c2nnc(Nc3cccc(C#N)c3)c3ccccc23)cc1] | E |
| 55 | 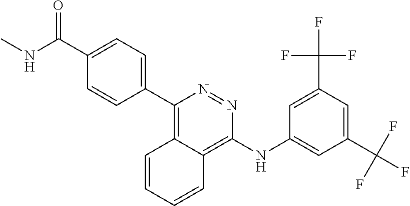 [SMILES: CNC(=O)c1ccc(-c2nnc(Nc3cc(C(F)(F)F)cc(C(F)(F)F)c3)c3ccccc23)cc1] | B |
| 56 | 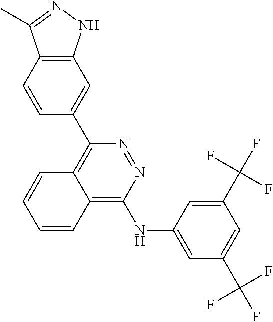 [SMILES: Cc1n[nH]c2cc(-c3nnc(Nc4cc(C(F)(F)F)cc(C(F)(F)F)c4)c4ccccc34)ccc12] | B |
| 57 | 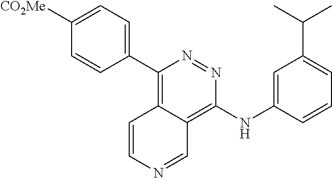 [SMILES: COC(=O)c1ccc(-c2nnc(Nc3cccc(C(C)C)c3)c3cnccc23)cc1] | C |
| 58 | 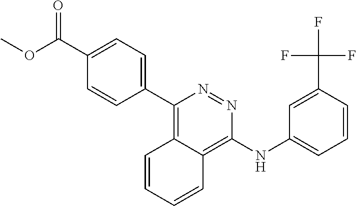 [SMILES: COC(=O)c1ccc(-c2nnc(Nc3cccc(C(F)(F)F)c3)c3ccccc23)cc1] | A |
| 59 | 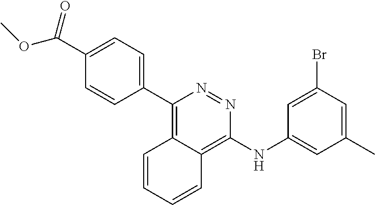 | B |
| 60 | 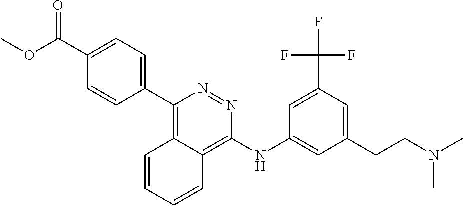 | C |
| 61 | 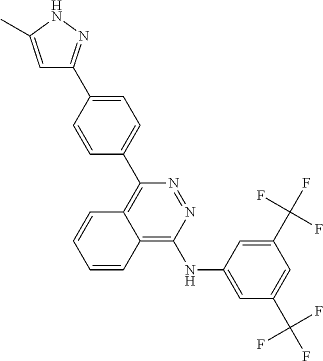 [SMILES: Cc1cc(-c2ccc(-c3nnc(Nc4cc(C(F)(F)F)cc(C(F)(F)F)c4)c4ccccc34)cc2)n[nH]1] | C |
| 62 | 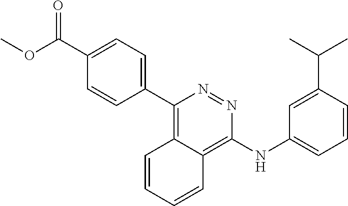 [SMILES: COC(=O)c1ccc(-c2nnc(Nc3cccc(C(C)C)c3)c3ccccc23)cc1] | B |
| 63 | 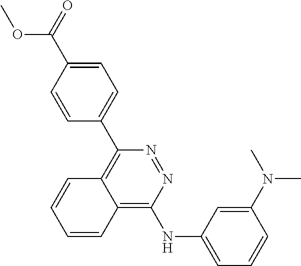 | E |
| 64 | 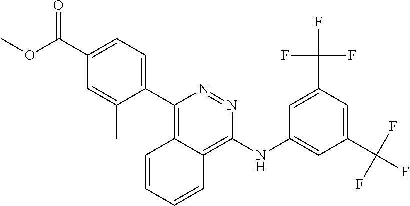 [SMILES: COC(=O)c1ccc(-c2nnc(Nc3cc(C(F)(F)F)cc(C(F)(F)F)c3)c3ccccc23)c(C)c1] | C |
| 65 | 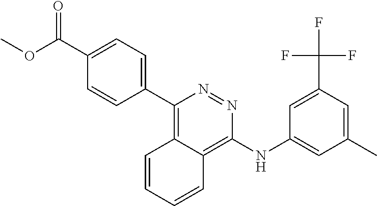 | A |
| 66 | 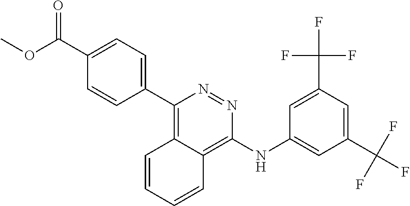 [SMILES: COC(=O)c1ccc(-c2nnc(Nc3cc(C(F)(F)F)cc(C(F)(F)F)c3)c3ccccc23)cc1] | B |

From the foregoing it will be appreciated that, although specific embodiments of this disclosure have been described herein for purposes of illustration, various modifications may be made without deviating from the spirit and scope of the invention. Accordingly, the invention is not limited except as by the appended claims.

1. A compound according to Formula I: 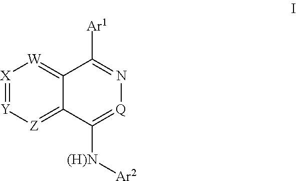
or a pharmaceutically acceptable salt thereof, wherein:
Ar¹ is phenyl optionally substituted with 1-2 R¹ groups or optionally fused to a 5-6 membered heterocyclyl, heterocyclyl, or heteroaryl optionally substituted with 1-2 R² groups;
Ar² is phenyl optionally substituted with 1-3 R⁵ groups;
each R¹ is independently halo, alkyl, —C(O)OR³, —C(O)R³, —C(O)N(H)alkylR⁴, —N(H)C(O)alkyl, —C(O)N(R³)(R⁴), —SO₂R³, heteroaryl optionally substituted with R³ or —NR³R⁷, or heterocyclyl substituted with oxo;
each R² is independently —N(R³)(R⁴), -alkylN(R3)(R⁴), oxo, alkyl, —C(O)R³, or —C(O)OR³;
R³ is H or alkyl;
R⁴ is H or alkyl optionally substituted with heterocyclyl;
each R⁵ is independently halo, —CN, —C(O)OR³, R⁶, —OR⁶, —N(R³)R⁶, alkyl optionally substituted with 1-3 halo, alkoxy optionally substituted with 1-3 halo, or heterocyclyl optionally substituted with R³;
R⁶ is alkyl optionally substituted with —NR³R⁷;
R⁷ is H or alkyl;
Q is C(H) or N;
W is C(H) or N;
X is C(H) or N;
Y is C(H) or N; and
Z is C(H) or N;
provided the compound is not

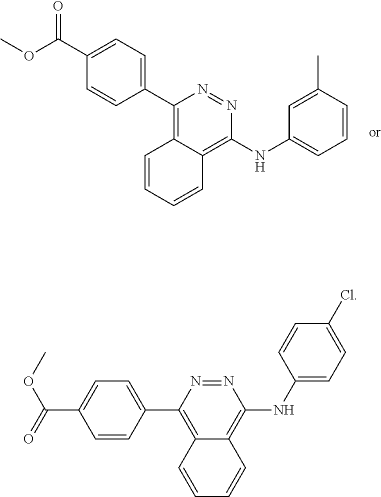 [SMILES: COC(=O)c1ccc(-c2nnc(Nc3cccc(C)c3)c3ccccc23)cc1 ; COC(=O)c1ccc(-c2nnc(Nc3ccc(Cl)cc3)c3ccccc23)cc1]

2. The compound according to claim 1 wherein:
Ar¹ is phenyl optionally substituted with 1-2 R¹ groups, or heteroaryl optionally substituted with 1-2 R² groups;
Ar² is phenyl optionally substituted with 1-3 R⁵ groups;
each R¹ is independently —C(O)OR³, —C(O)R³, —C(O)N(H)alkylR⁴, —N(H)C(O)alkyl, —C(O)N(R³)(R⁴), or —SO₂R³;
each R² is independently —N(R³)(R⁴), alkyl, or —C(O)OR³;
R³ is H or alkyl;
R⁴ is H or alkyl optionally substituted with heterocyclyl;
each R⁵ is independently halo, —CN, —C(O)OR³, R⁶, —OR⁶, —N(R³)R⁶, alkyl optionally substituted with 1-3 halo, alkoxy optionally substituted with 1-3 halo, or heterocyclyl optionally substituted with R³;
R⁶ is alkyl optionally substituted with —NR³R⁷;
R⁷ is H or alkyl;
Q is C(H) or N;
W is C(H) or N;
X is C(H) or N;
Y is C(H) or N; and
Z is C(H) or N.

3. The compound according to claim 1 wherein
Ar¹ is phenyl optionally substituted with 1-2 R¹ groups or heteroaryl optionally substituted with 1-2 R² groups;
Ar² is phenyl optionally substituted with 1-3 R⁵ groups;
each R¹ is independently —C(O)OR³, —C(O)R³, —C(O)N(H)alkylR⁴; —N(H)C(O)alkyl, —C(O)N(R³)(R⁴), —SO₂R³, heteroaryl optionally substituted with —NR³R⁷, or heterocyclyl substituted with oxo;
each R² is independently —N(R³)(R⁴), oxo, alkyl, —C(O)R³, or —C(O)OR³;
R³ is H or (C₁-C₃)alkyl;
R⁴ is H or (C₁-C₃)alkyl optionally substituted with a 5-6 membered heterocyclyl;
each R⁵ is independently halo, —CN, —C(O)OR³, R⁶, —OR⁶, —N(R³)R⁶, alkyl optionally substituted with 1-3 halo, alkoxy optionally substituted with 1-3 halo, or heterocyclyl optionally substituted with R³;
R⁶ is alkyl optionally substituted with —NR³R⁷;
R⁷ is H or alkyl;
Q is C(H) or N;
W is C(H) or N;
X is C(H) or N;
Y is C(H) or N; and
Z is C(H) or N;
provided that only one of W, X, Y or Z can be N.

4. The compound according to claim 3, wherein:
Ar¹ is phenyl optionally substituted with 1-2 R¹ groups or heteroaryl optionally substituted with 1-2 R² groups, wherein the heteroaryl is 1H-indazolyl, pyrazolyl, benzotriazolyl, or benzofuranyl, isoindolyl;
each R⁵ is independently halo, —CN, —C(O)OR³, R⁶, —OR⁶, —N(R³)R⁶, (C₁-C₃)alkyl optionally substituted with 1-3 halo, alkoxy optionally substituted with 1-3 halo, or heterocyclyl optionally substituted with R³; and
R⁷ is H or (C₁-C₃)alkyl.

5. The compound according to claim 1, wherein Ar² is: 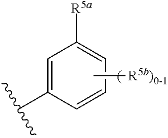
and wherein:
R^{5a} is selected from halo, —CN, —C(O)OR³, R⁶, —OR⁶, —N(R³)R⁶, (C₁-C₃)alkyl optionally substituted with 1-3 halo, alkoxy optionally substituted with 1-3 halo, and heterocyclyl optionally substituted with R³; and
R^{5b}, when present, is selected from halo, —CN, —C(O)OR³, R⁶, —OR⁶, —N(R³)R⁶, (C₁-C₃)alkyl optionally substituted with 1-3 halo, alkoxy optionally substituted with 1-3 halo, and heterocyclyl optionally substituted with R³.

6. The compound according to claim 1, wherein Ar² is: 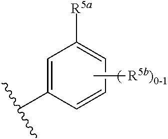
and wherein:
R^{5a} is selected from halo, —CN, —C(O)OR³, R⁶, —OR⁶, —N(R³)R⁶, (C₁-C₃)alkyl optionally substituted with 1-3 halo, alkoxy optionally substituted with 1-3 halo, and heterocyclyl optionally substituted with R³; and
R^{5b}, when present, is selected from halo, —CN, —C(O)OR³, R⁶, —OR⁶, —N(R³)R⁶, (C₁-C₃)alkyl optionally substituted with 1-3 halo, and alkoxy.

7. The compound according to claim 1, wherein Ar² is: 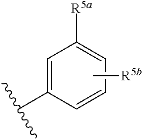
and wherein:
R^{5a} is selected from halo, —CN, —C(O)OR³, R⁶, —OR⁶, —N(R³)R⁶, (C₁-C₃)alkyl optionally substituted with 1-3 halo, alkoxy optionally substituted with 1-3 halo, and heterocyclyl optionally substituted with R³; and
R^{5b} is selected from halo, —CN, —C(O)OR³, R⁶, —OR⁶, —N(R³)R⁶, (C₁-C₃)alkyl optionally substituted with 1-3 halo, and alkoxy.

8. The compound according to claim 5 having Formula IA:

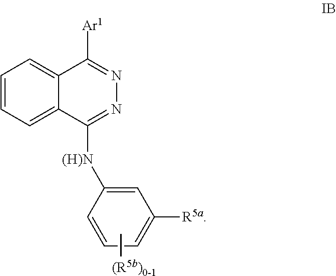

9. The compound according to claim 1 wherein Ar¹ is

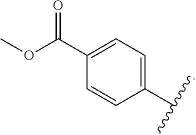 [SMILES: *c1ccc(C(=O)OC)cc1]

10. The compound according to claim 9 wherein Ar² is 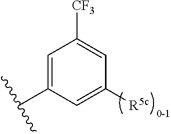
and wherein R^{5C}, when present, is halo, alkyl, or —N(R³)R⁶.

11. A compound according to claim 1 selected from the group consisting of:
N-[3,5-bis(trifluoromethyl)phenyl]-4-{4-[5-(methylamino)-1,3,4-
thiadiazol-2-yl]phenyl}phthalazin-1-amine;
methyl 4-(4-{[3-(trifluoromethyl)phenyl]amino}isoquinolin-1-
yl)benzoate;
ethyl 6-(4-{[3,5-bis(trifluoromethyl)phenyl]amino}phthalazin-1-yl)-
1H-indazole-3-carboxylate;
methyl 4-(4-{[3-(1,1-dimethylethyl)phenyl]amino}phthalazin-1-
yl)benzoate;
1-[4-(4-{[3,5-bis(trifluoromethyl)phenyl]amino}phthalazin-1-
yl)phenyl]-1,3-dihydro-2H-imidazol-2-one;
methyl 4-(4-{[3,5-bis(trifluoromethyl)phenyl]amino}phthalazin-1-
yl)-3-fluorobenzoate;
N-[3,5-bis(trifluoromethyl)phenyl]-4-{3-[(methylamino)methyl]-
1H-indazol-6-yl}phthalazin-1-amine;
methyl 4-(4-{[3-{[3-(dimethylamino)propyl]amino}-5-
(trifluoromethyl)phenyl]amino}phthalazin-1-yl)benzoate;
4-(4-{[3,5-bis(trifluoromethyl)phenyl]amino}phthalazin-1-
yl)benzamide;
methyl 4-(4-{[3-{[2-(dimethylamino)ethyl]oxy}-5-
(trifluoromethyl)phenyl]amino}phthalazin-1-yl)benzoate;
methyl 4-(4-{[3,5-bis(trifluoromethyl)phenyl]amino}phthalazin-1-
yl)-3-chlorobenzoate;
1-[5-(4-{[3,5-bis(trifluoromethyl)phenyl]amino}phthalazin-1-yl)-2-
thienyl]ethanone;
methyl 4-(5-{[3-(trifluoromethyl)phenyl]amino}pyrido[2,3-
d]pyridazin-8-yl)benzoate;
methyl 4-(4-{[3-chloro-5-
(trifluoromethyl)phenyl]amino}phthalazin-1-yl)benzoate;
methyl 3-[(4-(4-[(methyloxy)carbonyl]phenyl}phthalazin-1-
yl)amino]-5-(trifluoromethyl)benzoate;
methyl 4-{4-[(3-chlorophenyl)amino]phthalazin-1-yl}benzoate;
methyl 5-(4-{[3,5-bis(trifluoromethyl)phenyl]amino}phthalazin-1-
yl)thiophene-2-carboxylate;
4-(4-{[3,5-bis(trifluoromethyl)phenyl]amino}phthalazin-1-
yl)benzaldehyde;
methyl 4-(4-{[3-ethyl-5-(trifluoromethyl)phenyl]amino}phthalazin-
1-yl)benzoate;
methyl 4-(4-{[3-(4-methylpiperazin-1-yl)-5-
(trifluoromethyl)phenyl]amino}phthalazin-1-yl)benzoate;
methyl 4-{4-[(3-ethylphenyl)amino]phthalazin-1-yl}benzoate;
methyl 4-[4-({3-[3-(dimethylamino)propyl]-5-
(trifluoromethyl)phenyl}amino)phthalazin-1-yl]benzoate;
1-[4-(4-{[3,5-bis(trifluoromethyl)phenyl]amino}phthalazin-1-
yl)phenyl]imidazolidin-2-one;
methyl 4-(4-{[4-chloro-3-
(trifluoromethyl)phenyl]amino}phthalazin-1-yl)benzoate;
methyl 4-(4-{[3-(1-methylethyl)phenyl]amino}isoquinolin-1-
yl)benzoate;
5-(4-{[3,5-bis(trifluoromethyl)phenyl]amino}phthalazin-1-yl)-2-
benzofuran-1(3H)-one;
N-[4-(4-{[3,5-bis(trifluoromethyl)phenyl]amino}phthalazin-1-
yl)phenyl]acetamide;
methyl 4-(4-{[3-(trifluoromethyl)phenyl]amino}pyrido[3,4-
d]pyridazin-1-yl)benzoate;
methyl 4-[4-({3-[(dimethylamino)methyl]-5-
(trifluoromethyl)phenyl}amino)phthalazin-1-yl]benzoate;
methyl 4-(4-{[3-(1-methylpiperidin-4-yl)-5-
(trifluoromethyl)phenyl]amino}phthalazin-1-yl)benzoate;
methyl 4-(4-{[3-(methyloxy)-5-
(trifluoromethyl)phenyl]amino}phthalazin-1-yl)benzoate;
N-[3,5-bis(trifluoromethyl)phenyl]-4-(1H-indazol-6-yl)phthalazin-1-
amine;
methyl 4-(4-{[4-(methyloxy)-3-
(trifluoromethyl)phenyl]amino}phthalazin-1-yl)benzoate;
methyl 4-(4-{[3-fluoro-5-(trifluoromethyl)phenyl]amino}phthalazin-
1-yl)benzoate;
methyl 4-(4-{[3-{[2-(dimethylamino)ethyl]amino}-5-
(trifluoromethyl)phenyl]amino}phthalazin-1-yl)benzoate;
N-{3-[3-(dimethylamino)propyl]-5-(trifluoromethyl)phenyl}-4-(3-
methyl-1H-indazol-6-yl)phthalazin-1-amine;
4-(3-amino-1H-indazol-6-yl)-N-[3,5-
bis(trifluoromethyl)phenyl]phthalazin-1-amine;
methyl 4-(8-{[3-(trifluoromethyl)phenyl]amino}pyrido[2,3-
d]pyridazin-5-yl)benzoate;
4-(4-{[3,5-bis(trifluoromethyl)phenyl]amino}phthalazin-1-
yl)benzoic acid;
N-[3,5-bis(trifluoromethyl)phenyl]-4-(1,3-dihydro-2H-pyrrolo[3,4-
c]pyridin-2-yl)phthalazin-1-amine;
4-[4-(4-{[3,5-bis(trifluoromethyl)phenyl]amino}phthalazin-1-
yl)phenyl]-2,4-dihydro-3H-1,2,4-triazol-3-one;
methyl 4-[4-({3-[(trifluoromethyl)oxy]phenyl}amino)phthalazin-1-
yl]benzoate;
methyl 4-(4-{[3-bromo-5-
(trifluoromethyl)phenyl]amino}phthalazin-1-yl)benzoate;
4-(1H-1,2,3-benzotriazol-6-yl)-N-[3,5-
bis(trifluoromethyl)phenyl]phthalazin-1-amine;
N-[3,5-bis(trifluoromethyl)phenyl]-4-(3-ethyl-1H-indazol-6-
yl)phthalazin-1-amine;
1-[4-(4-{[3,5-bis(trifluoromethyl)phenyl]amino}phthalazin-1-
yl)phenyl]pyrrolidin-2-one;
4-(4-{[3,5-bis(trifluoromethyl)phenyl]amino}phthalazin-1-yl)-N-(2-
morpholin-4-ylethyl)benzamide;
1-[3-(4-{[3,5-bis(trifluoromethyl)phenyl]amino}phthalazin-1-
yl)phenyl]ethanone;
methyl 4-{4-[(3-bromophenyl)amino]phthalazin-1-yl}benzoate;
N-[3,5-bis(trifluoromethyl)phenyl]-4-[4-(1H-pyrazol-3-
yl)phenyl]phthalazin-1-amine;
methyl 4-(4-{[4-fluoro-3-(trifluoromethyl)phenyl]amino}phthalazin-
1-yl)benzoate;
methyl 4-{4-[(3-cyanophenyl)amino]phthalazin-1-yl}benzoate;
4-(4-{[3,5-bis(trifluoromethyl)phenyl]amino}phthalazin-1-yl)-N-
methylbenzamide;
N-[3,5-bis(trifluoromethyl)phenyl]-4-(3-methyl-1H-indazol-6-
yl)phthalazin-1-amine;
methyl 4-(4-{[3-(1-methylethyl)phenyl]amino}pyrido[3,4-
d]pyridazin-1-yl)benzoate;
methyl 4-(4-{[3-(trifluoromethyl)phenyl]amino}phthalazin-1-
yl)benzoate;
methyl 4-{4-[(3-bromo-5-methylphenyl)amino]phthalazin-1-
yl}benzoate;
methyl 4-[4-({3-[2-(dimethylamino)ethyl]-5-
(trifluoromethyl)phenyl}amino)phthalazin-1-yl]benzoate;
N-[3,5-bis(trifluoromethyl)phenyl]-4-[4-(5-methyl-1H-pyrazol-3-
yl)phenyl]phthalazin-1-amine;
methyl 4-(4-{[3-(1-methylethyl)phenyl]amino}phthalazin-1-
yl)benzoate;
methyl 4-(4-{[3-(dimethylamino)phenyl]amino}phthalazin-1-
yl)benzoate;
methyl 4-(4-{[3,5-bis(trifluoromethyl)phenyl]amino}phthalazin-1-
yl)-3-methylbenzoate;
methyl 4-(4-{[3-methyl-5-
(trifluoromethyl)phenyl]amino}phthalazin-1-yl)benzoate; and
methyl 4-(4-{[3,5-bis(trifluoromethyl)phenyl]amino}phthalazin-1-
yl)benzoate;
or a pharmaceutically acceptable salt of any of the above compounds.

12. A composition comprising a compound according to claim 1 and a pharmaceutically acceptable diluent, excipient, or carrier.

13. A method for inhibiting JAK1 comprising administering a compound according to claim 1 or

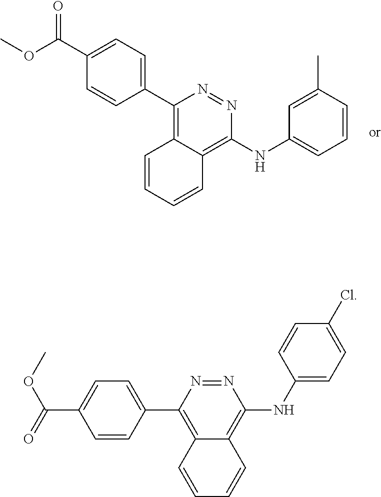 [SMILES: COC(=O)c1ccc(-c2nnc(Nc3cccc(C)c3)c3ccccc23)cc1 ; COC(=O)c1ccc(-c2nnc(Nc3ccc(Cl)cc3)c3ccccc23)cc1]

14. A method for treating a disease JAK1 mediates or is implicated in a subject in need thereof comprising administrating to the subject a therapeutically effective amount of a compound according to claim 1 or

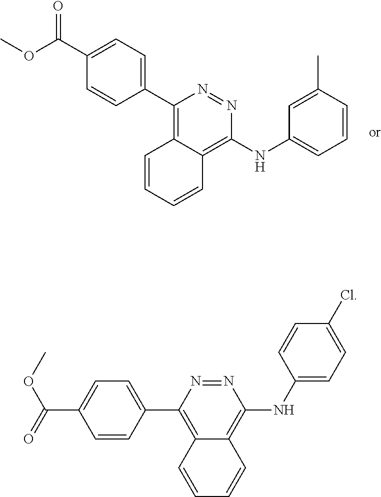 [SMILES: COC(=O)c1ccc(-c2nnc(Nc3cccc(C)c3)c3ccccc23)cc1 ; COC(=O)c1ccc(-c2nnc(Nc3ccc(Cl)cc3)c3ccccc23)cc1]

15. The method of claim 14 wherein the disease is selected from the group consisting of cancer, inflammatory disorders, and autoimmune diseases.
